(19) United States
(12) Patent Application Publication
(10) Pub. No.: US 2024/0182556 A1
(43) Pub. Date: Jun. 6, 2024

(54) STEM CELL FACTOR ANTIBODIES AND METHODS OF USE THEREOF

(71) Applicant: OPSIDIO, LLC, Bryn Mawr, PA (US)

(72) Inventors: Andrew R.M. BRADBURY, Santa Fe, NM (US); Andre TEIXEIRA, Santa Fe, NM (US); Camila LEAL, Santa Fe, NM (US)

(21) Appl. No.: 18/550,438

(22) PCT Filed: Mar. 17, 2022
(86) PCT No.: PCT/US2022/020732

§ 371 Date: Sep. 13, 2023

Related U.S. Application Data
(60) Provisional application No. 63/162,322, filed on Mar. 17, 2021.

(51) Int. Cl. C07K 16/24; A61K 39/00; A61P 37/06

(52) U.S. Cl. CPC C07K 16/24; A61P 37/06; A61K 39/00

(57) ABSTRACT

The disclosure relates to antibodies and antigen-binding fragments thereof that bind to Stem Cell Factor (SCF) with high affinity. The antibodies and antigen-binding fragments thereof specifically bind to SCF248 with high affinity. The disclosure further relates to methods of use of the antibodies including methods of treatment for inflammatory and/or fibrotic diseases and disorders.

### CROSS-REFERENCE TO RELATED APPLICATIONS

This application claims priority to U.S. Provisional Application No. 63/162,322, filed on Mar. 17, 2021. This application is hereby incorporated by reference in its entirety for all purposes.

### FIELD

The present invention relates to antibodies and antigen-binding fragments thereof that bind to Stem Cell Factor (SCF) and particular portions thereof, and to methods of using such antibodies and antigen-binding fragments.

### DESCRIPTION OF THE TEXT FILE SUBMITTED ELECTRONICALLY

The contents of the text file submitted electronically herewith are incorporated herein by reference in their entirety: A computer readable format copy of the Sequence Listing (filename: OPSL_002_01WO_SeqList_ST25.txt, date recorded: Mar. 9, 2022, file size 2.38 megabytes).

### BACKGROUND

Inflammatory diseases are a major cause of morbidity and mortality worldwide. Some types of chronic inflammation can lead to fibrosis, which is the formation or development of excess fibrous connective tissue in an organ or tissue as a reparative or reactive process, as opposed to formation of fibrous tissue as a normal constituent of an organ or tissue. Chronic inflammation as well as fibrosis can affect nearly all tissues and organ systems, and fibrotic tissue remodeling can influence cancer metastasis and accelerate chronic graft rejection in transplant recipients.

Stem cell factor (SCF) and its receptor c-Kit are important factors of the perpetuation of chronic inflammation and in fibrotic diseases (El-Koraie, et al., Kidney Int. 60: 167 (2001); Powell, et al., Am. J. Physiol. 289: G2(2005); El Kossi, et al., Am. J. Kidney Dis. 41: 785 (2003); Powell, et al., Am. J. Physiol. 277: C183 (1999) Ding et al J Pathol. 2013 June; 230(2):205-14., Berlin et al Lab Invest. 2006 June; 86(6):557-65, Rasky et al Am J Physiol Lung Cell Mol Physiol. 2020 Jan. 1; 318(1):L200-L211). c-Kit is a type Ill receptor-tyrosine kinase that is present in many cell types (Orr-Urtreger et al., Development 109: 911 (1990). Immune cells such as mast cells, eosinophils, and innate lymphoid cells 2 and 3 (ILC2 and ILC3) are all c-Kit+ cells that may drive the chronic inflammatory process, depending on the disease and organ involved. Upon initiation of an inflammatory response, various mediators, including SCF, activate c-Kit+ immune cells, which in turn produce cytokines that cause fibroblasts to become activated myofibroblasts. Myofibroblasts secrete extracellular matrix proteins, collagen, and fibronectin, resulting in fibrosis of tissue. Activated myofibroblasts, activated epithelia, endothelia, macrophages, eosinophils, mast cells, monocytes, and other cells also express SCF on the cell surface, which activates more c-Kit+ immune cells, resulting in more cytokine release and perpetuating the inflammation.

There is a need in the art for more efficient and more specific treatments for inflammatory diseases. In particular, there is a need for improved treatments for inflammatory diseases in humans. The present disclosure addresses this and other needs.

### SUMMARY OF THE DISCLOSURE

In some embodiments, provided herein is an antibody or fragment thereof that specifically binds to stem cell factor (SCF), wherein the antibody comprises a heavy chain and a light chain, the heavy and light chain each comprising three complementarity determining regions (CDRs), comprising: (i) a heavy chain CDR1 comprising an amino acid sequence according to SEQ ID NO: 1 (SX₂X₃MN, wherein X₂ is Q, N, or Y; and X₃ is W or Y); (ii) a heavy chain CDR2 comprising an amino acid sequence according to SEQ ID NO: 2 (QIYPX₅DX₇DX₉HX₁₁NX₁₃KFX₁₆X₁₇, wherein X₅ is E, G, D, or L; X₇ is G, D or N; X₉ is T or I; X₁₁ is M or Y; X₁₃ is G, D, or E; X₁₆ is K, R, N, E, or D; and X₁₇ is G or T); (iii) a heavy chain CDR3 comprising an amino acid sequence according to SEQ ID NO: 3 (X₁NWX₄GSY, wherein X₁ is S or A; and X₄ is V or D); (iv) a light chain CDR1 comprising an amino acid sequence according to SEQ ID NO: 4 (X₁X₂SQSLLX₈X₉DGNTYLN, wherein X₁ is K or H; X₂ is S or A; X₈ is E or D; and X₉ is S, E, Q, A, or G); (v) a light chain CDR2 comprising an amino acid sequence according to SEQ ID NO: 5 (LVX₃RX₅DX₇, wherein X₃ is D, N, or S; X₅ is L or R; and X₇ is I, D, S, or L); and (vi) a light chain CDR3 comprising an amino acid sequence according to SEQ ID NO: 6 (WQGX₄X₅LPQT, wherein X₄ is T or S; and X₅ is D or H).

In some embodiments, provided herein is an antibody or fragment thereof, comprising. (i) a heavy chain CDR1 comprising an amino acid sequence according to SEQ ID NO: 7 (SX₂WMN, wherein X₂ is Q or N); (ii) a heavy chain CDR2 comprising an amino acid sequence according to SEQ ID NO: 8 (QIYPX₅DX₇DX₉HX₁₁NX₁₃KFKX₁₇, wherein X₅ is E, G, or D; X₇ is G or D; X₉ is T or I; X₁₃ is G or D, X₁₇ is G or T, Xn is M or Y); (iii) a heavy chain CDR3 comprising an amino acid sequence according to SEQ ID NO: 9 (SNWX₄GSY, wherein X₄ is V or D); (iv) a light chain CDR1 comprising an amino acid sequence according to SEQ ID NO: 10 (KSSQSLLEX₉DGNTYLN, wherein X₉ is S, E, Q, or A); (v) a light chain CDR2 comprising an amino acid sequence according to SEQ ID NO: 11 (LVX₃RLDX₇, wherein X; is D or N; X₇ is I, D, S, or L); and (vi) a light chain CDR3 according to SEQ ID NO: 6 (WQGX₄X₅LPQT, wherein X₄ is T or S; and X₅ is D or H).

In some embodiments, provided herein is an antibody or fragment thereof, comprising: (i) a heavy chain CDR1, CDR2, and CDR3 according to SEQ ID Nos: 22, 26, and 16, respectively; and a light chain CDR1, CDR2, and CDR3 according to SEQ ID NOs: 71, 90, and 111; (ii) a heavy chain CDR1, CDR2, and CDR3 according to SEQ ID Nos: 23, 27, and 16, respectively; and a light chain CDR1, CDR2, and CDR3 according to SEQ ID NOs: 71, 91, and 111; (iii) a heavy chain CDR1, CDR2, and CDR3 according to SEQ ID Nos: 22, 26, and 67, respectively; and a light chain CDR1, CDR2, and CDR3 according to SEQ ID NOs: 71, 92, and 111; (iv) a heavy chain CDR1, CDR2, and CDR3 according to SEQ ID Nos: 22, 27, and 16, respectively; and a light chain CDR1, CDR2, and CDR3 according to SEQ ID NOs: 71, 92, and 111; (v) a heavy chain CDR1, CDR2, and CDR3 according to SEQ ID Nos. 23, 28, and 16, respectively; and a light chain CDR1, CDR2, and CDR3 according to SEQ ID NOs: 72, 92, and 111; (vi) a heavy chain CDR1, CDR2, and CDR3 according to SEQ ID Nos: 22, 27, and 16, respectively; and a light chain CDR1, CDR2, and CDR3 according to SEQ ID NOs: 71, 93, and 112; (vii) a heavy chain CDR1, CDR2, and CDR3 according to SEQ ID Nos: 22, 28, and 16, respectively; and a light chain CDR1, CDR2, and CDR3 according to SEQ ID NOs: 73, 92, and 111; (viii) a heavy chain CDR1, CDR2, and CDR3 according to SEQ ID Nos: 22, 29, and 16, respectively; and a light chain CDR1, CDR2, and CDR3 according to SEQ ID NOs: 73, 92, and 111; (ix) a heavy chain CDR1, CDR2, and CDR3 according to SEQ ID Nos: 22, 30, and 16, respectively; and a light chain CDR1, CDR2, and CDR3 according to SEQ ID NOs: 71, 92, and 111; (x) a heavy chain CDR1, CDR2, and CDR3 according to SEQ ID Nos: 22, 31, and 16, respectively; and a light chain CDR1, CDR2, and CDR3 according to SEQ ID NOs: 71, 92, and 112; (xi) a heavy chain CDR1, CDR2, and CDR3 according to SEQ ID Nos: 22, 32, and 16, respectively; and a light chain CDR1, CDR2, and CDR3 according to SEQ ID NOs: 73, 93, and 111; (xii) a heavy chain CDR1, CDR2, and CDR3 according to SEQ ID Nos: 22, 33, and 16, respectively; and a light chain CDR1, CDR2, and CDR3 according to SEQ ID NOs: 74, 92, and 111; (xiii) a heavy chain CDR1, CDR2, and CDR3 according to SEQ ID Nos: 24, 34, and 16, respectively; and a light chain CDR1, CDR2, and CDR3 according to SEQ ID NOs: 73, 94, and 111; (xiv) a heavy chain CDR1, CDR2, and CDR3 according to SEQ ID Nos: 23, 28, an d16, respectively; and a light chain CDR1, CDR2, and CDR3 according to SEQ ID NOs: 73, 94, and 111: (xv) a heavy chain CDR1, CDR2, and CDR3 according to SEQ ID Nos: 22, 31, and 16, respectively; and a light chain CDR1. CDR2, and CDR3 according to SEQ ID NOs: 73, 92, and 111; (xvi) a heavy chain CDR1, CDR2, and CDR3 according to SEQ ID Nos: 22, 28, and 16, respectively; and a light chain CDR I, CDR2, and CDR3 according to SEQ ID NOs: 73, 92, and 111; (xvii) a heavy chain CDR1, CDR2, and CDR3 according to SEQ ID Nos: 22, 27, and 16, respectively; and a light chain CDR1, CDR2, and CDR3 according to SEQ ID NOs: 73, 92, and 111; (xviii) a heavy chain CDR1, CDR2, and CDR3 according to SEQ ID Nos: 22, 35, and 16, respectively; and a light chain CDR1, CDR2, and CDR3 according to SEQ ID NOs: 74, 92, and 111; (xix) a heavy chain CDR1, CDR2, and CDR3 according to SEQ ID Nos: 22, 34, and 16, respectively; and a light chain CDR1, CDR2, and CDR3 according to SEQ ID NOs: 71, 92, and 111; (xx) a heavy chain CDR1, CDR2, and CDR3 according to SEQ ID Nos: 22, 27, and 16, respectively; and a light chain CDR1, CDR2, and CDR3 according to SEQ ID NOs: 75, 92, and 111; (xxi) a heavy chain CDR1, CDR2, and CDR3 according to SEQ ID Nos: 24, 34, and 16, respectively; and a light chain CDR1, CDR2, and CDR3 according to SEQ ID NOs: 73, 92, and 111; (xxii) a heavy chain CDR1, CDR2, and CDR3 according to SEQ ID Nos: 22, 36, and 16, respectively; and a light chain CDR1, CDR2, and CDR3 according to SEQ ID NOs: 71, 95, and 111: (xxiii) a heavy chain CDR1, CDR2, and CDR3 according to SEQ ID Nos: 23, 28, and 16, respectively; and a light chain CDR1, CDR2, and CDR3 according to SEQ ID NOs: 73, 92, and 111; (xxiv) a heavy chain CDR1, CDR2, and CDR3 according to SEQ ID Nos: 22, 28, and 16, respectively; and a light chain CDR1, CDR2, and CDR3 according to SEQ ID NOs: 73, 96, and 111; (xxv) a heavy chain CDR1, CDR2, and CDR3 according to SEQ ID Nos: 22, 34, and 16, respectively; and a light chain CDR1, CDR2, and CDR3 according to SEQ ID NOs: 71, 95, and 111; (xxvi) a heavy chain CDR1, CDR2, and CDR3 according to SEQ ID Nos: 22, 34, and 16, respectively; and a light chain CDR1, CDR2, and CDR3 according to SEQ ID NOs: 79, 90, and 111; (xxvii) a heavy chain CDR1, CDR2, and CDR3 according to SEQ ID Nos: 22, 34, and 16, respectively; and a light chain CDR1, CDR2, and CDR3 according to SEQ ID NOs: 75, 92, and 111: or (xxviii) a heavy chain CDR1, CDR2, and CDR3 according to SEQ ID Nos: 22, 27, and 16, respectively; and a light chain CDR1, CDR2, and CDR3 according to SEQ ID NOs: 73, 98, and 111.

In some embodiments, provided herein is an antibody or fragment thereof, wherein the antibody comprises: (i) a heavy chain variable region comprising an amino acid sequence having at least 80% identity to SEQ ID NO: 114 and a light chain variable region comprising an amino acid sequence having at least 80% identity to SEQ ID NO: 291; (ii) a heavy chain variable region comprising an amino acid sequence having at least 80% identity to SEQ ID NO: 115 and a light chain variable region comprising an amino acid sequence having at least 80% identity to SEQ ID NO: 292; (iii) a heavy chain variable region comprising an amino acid sequence having at least 80% identity to SEQ ID NO: 116 and a light chain variable region comprising an amino acid sequence having at least 80% identity to SEQ ID NO: 293; (iv) a heavy chain variable region comprising an amino acid sequence having at least 80% identity to SEQ ID NO: 117 and a light chain variable region comprising an amino acid sequence having at least 80% identity to SEQ ID NO: 294; (v) a heavy chain variable region comprising an amino acid sequence having at least 80% identity to SEQ ID NO: 118 and a light chain variable region comprising an amino acid sequence having at least 80%/c identity to SEQ ID NO: 295; (vi) a heavy chain variable region comprising an amino acid sequence having at least 80% identity to SEQ ID NO: 119 and a light chain variable region comprising an amino acid sequence having at least 80% identity to SEQ ID NO: 296; (vii) a heavy chain variable region comprising an amino acid sequence having at least 80% identity to SEQ ID NO: 120 and a light chain variable region comprising an amino acid sequence having at least 80% identity to SEQ ID NO: 297; (viii) a heavy chain variable region comprising an amino acid sequence having at least 80% identity to SEQ ID NO: 121 and a light chain variable region comprising an amino acid sequence having at least 80% identity to SEQ ID NO: 298; (ix) a heavy chain variable region comprising an amino acid sequence having at least 80% identity to SEQ ID NO: 122 and a light chain variable region comprising an amino acid sequence having at least 80% identity to SEQ ID NO: 299; (x) a heavy chain variable region comprising an amino acid sequence having at least 80% identity to SEQ ID NO: 123 and a light chain variable region comprising an amino acid sequence having at least 80% identity to SEQ ID NO: 300; (xi) a heavy chain variable region comprising an amino acid sequence having at least 80% identity to SEQ ID NO: 124 and a light chain variable region comprising an amino acid sequence having at least 80% identity to SEQ ID NO: 301; (xii) a heavy chain variable region comprising an amino acid sequence having at least 80% identity to SEQ ID NO: 125 and a light chain variable region comprising an amino acid sequence having at least 80% identity to SEQ ID NO: 302; (xiii) a heavy chain variable region comprising an amino acid sequence having at least 80% identity to SEQ ID NO: 126 and a light chain variable region comprising an amino acid sequence having at least 80% identity to SEQ ID NO: 303; (xiv) a heavy chain variable region comprising an amino acid sequence having at least 80% identity to SEQ ID NO: 127 and a light chain variable region comprising an amino acid sequence having at least 80% identity to SEQ ID NO: 304; (xv) a heavy chain variable region comprising an amino acid sequence having at least 80% identity to SEQ ID NO: 128 and a light chain variable region comprising an amino acid sequence having at least 80% identity to SEQ ID NO: 305; (xvi) a heavy chain variable region comprising an amino acid sequence having at least 80% identity to SEQ ID NO: 129 and a light chain variable region comprising an amino acid sequence having at least 80% identity to SEQ ID NO: 306; (xvii) a heavy chain variable region comprising an amino acid sequence having at least 80% identity to SEQ ID NO: 130 and a light chain variable region comprising an amino acid sequence having at least 80% identity to SEQ ID NO: 307; (xviii) a heavy chain variable region comprising an amino acid sequence having at least 80% identity to SEQ ID NO: 131 and a light chain variable region comprising an amino acid sequence having at least 80% identity to SEQ ID NO: 308; (xix) a heavy chain variable region comprising an amino acid sequence having at least 80% identity to SEQ ID NO: 132 and a light chain variable region comprising an amino acid sequence having at least 80% identity to SEQ ID NO: 309; (xx) a heavy chain variable region comprising an amino acid sequence having at least 80% identity to SEQ ID NO: 133 and a light chain variable region comprising an amino acid sequence having at least 80% identity to SEQ ID NO: 310; (xxi) a heavy chain variable region comprising an amino acid sequence having at least 80% identity to SEQ ID NO: 134 and a light chain variable region comprising an amino acid sequence having at least 80% identity to SEQ ID NO: 311; (xxii) a heavy chain variable region comprising an amino acid sequence having at least 80% identity to SEQ ID NO: 135 and a light chain variable region comprising an amino acid sequence having at least 80% identity to SEQ ID NO: 312; (xxiii) a heavy chain variable region comprising an amino acid sequence having at least 80% identity to SEQ ID NO: 136 and a light chain variable region comprising an amino acid sequence having at least 80% identity to SEQ ID NO: 313; (xxiv) a heavy chain variable region comprising an amino acid sequence having at least 80% identity to SEQ ID NO: 137 and a light chain variable region comprising an amino acid sequence having at least 80% identity to SEQ ID NO: 314; (xxv) a heavy chain variable region comprising an amino acid sequence having at least 80% identity to SEQ ID NO: 149 and a light chain variable region comprising an amino acid sequence having at least 80% identity to SEQ ID NO: 326; (xxvi) a heavy chain variable region comprising an amino acid sequence having at least 80% identity to SEQ ID NO: 156 and a light chain variable region comprising an amino acid sequence having at least 80% identity to SEQ ID NO: 333; (xxvii) a heavy chain variable region comprising an amino acid sequence having at least 80% identity to SEQ ID NO: 158 and a light chain variable region comprising an amino acid sequence having at least 80% identity to SEQ ID NO: 335; or (xxviii) a heavy chain variable region comprising an amino acid sequence having at least 80% identity to SEQ ID NO: 143 and a light chain variable region comprising an amino acid sequence having at least 80% identity to SEQ ID NO: 320.

In some embodiments, provided herein is an antibody or fragment thereof, wherein the antibody comprises: (i) a heavy chain variable region comprising an amino acid sequence having at least 90% identity to SEQ ID NO: 114 and a light chain variable region comprising an amino acid sequence having at least 90% identity to SEQ ID NO: 291; (ii) a heavy chain variable region comprising an amino acid sequence having at least 90% identity to SEQ ID NO: 115 and a light chain variable region comprising an amino acid sequence having at least 90% identity to SEQ ID NO: 292; (iii) a heavy chain variable region comprising an amino acid sequence having at least 90% identity to SEQ ID NO: 116 and a light chain variable region comprising an amino acid sequence having at least 90% identity to SEQ ID NO: 293; (iv) a heavy chain variable region comprising an amino acid sequence having at least 90% identity to SEQ ID NO: 117 and a light chain variable region comprising an amino acid sequence having at least 90% identity to SEQ ID NO: 294; (v) a heavy chain variable region comprising an amino acid sequence having at least 90% identity to SEQ ID NO: 118 and a light chain variable region comprising an amino acid sequence having at least 90% identity to SEQ ID NO: 295; (vi) a heavy chain variable region comprising an amino acid sequence having at least 90% identity to SEQ ID NO: 119 and a light chain variable region comprising an amino acid sequence having at least 90% identity to SEQ ID NO: 296; (vii) a heavy chain variable region comprising an amino acid sequence having at least 90% identity to SEQ ID NO: 120 and a light chain variable region comprising an amino acid sequence having at least 90% identity to SEQ ID NO: 297; (viii) a heavy chain variable region comprising an amino acid sequence having at least 90% identity to SEQ ID NO: 121 and a light chain variable region comprising an amino acid sequence having at least 90% identity to SEQ ID NO: 298; (ix) a heavy chain variable region comprising an amino acid sequence having at least 90% identity to SEQ ID NO: 122 and a light chain variable region comprising an amino acid sequence having at least 90% identity to SEQ ID NO: 299; (x) a heavy chain variable region comprising an amino acid sequence having at least 90% identity to SEQ ID NO: 123 and a light chain variable region comprising an amino acid sequence having at least 90% identity to SEQ ID NO: 300; (xi) a heavy chain variable region comprising an amino acid sequence having at least 90% identity to SEQ ID NO: 124 and a light chain variable region comprising an amino acid sequence having at least 90% identity to SEQ ID NO: 301; (xii) a heavy chain variable region comprising an amino acid sequence having at least 90% identity to SEQ ID NO: 125 and a light chain variable region comprising an amino acid sequence having at least 90% identity to SEQ ID NO: 302; (xiii) a heavy chain variable region comprising an amino acid sequence having at least 90% identity to SEQ ID NO: 126 and a light chain variable region comprising an amino acid sequence having at least 90% identity to SEQ ID NO: 303; (xiv) a heavy chain variable region comprising an amino acid sequence having at least 90% identity to SEQ ID NO: 127 and a light chain variable region comprising an amino acid sequence having at least 90% identity to SEQ ID NO: 304; (xv) a heavy chain variable region comprising an amino acid sequence having at least 90% identity to SEQ ID NO: 128 and a light chain variable region comprising an amino acid sequence having at least 90% identity to SEQ ID NO: 305; (xvi) a heavy chain variable region comprising an amino acid sequence having at least 90% identity to SEQ ID NO: 129 and a light chain variable region comprising an amino acid sequence having at least 90% identity to SEQ ID NO: 306; (xvii) a heavy chain variable region comprising an amino acid sequence having at least 90% identity to SEQ ID NO: 130 and a light chain variable region comprising an amino acid sequence having at least 90%/o identity to SEQ ID NO: 307; (xviii) a heavy chain variable region comprising an amino acid sequence having at least 90% identity to SEQ ID NO: 131 and a light chain variable region comprising an amino acid sequence having at least 90% identity to SEQ ID NO: 308; (xix) a heavy chain variable region comprising an amino acid sequence having at least 90% identity to SEQ ID NO: 132 and a light chain variable region comprising an amino acid sequence having at least 90% identity to SEQ ID NO: 309; (xx) a heavy chain variable region comprising an amino acid sequence having at least 90% identity to SEQ ID NO: 133 and a light chain variable region comprising an amino acid sequence having at least 90% identity to SEQ ID NO: 310; (xxi) a heavy chain variable region comprising an amino acid sequence having at least 90% identity to SEQ ID NO: 134 and a light chain variable region comprising an amino acid sequence having at least 90/o identity to SEQ ID NO: 311; (xxii) a heavy chain variable region comprising an amino acid sequence having at least 90% identity to SEQ ID NO: 135 and a light chain variable region comprising an amino acid sequence having at least 90% identity to SEQ ID NO: 312; (xxiii) a heavy chain variable region comprising an amino acid sequence having at least 90% identity to SEQ ID NO: 136 and a light chain variable region comprising an amino acid sequence having at least 90% identity to SEQ ID NO: 313; (xxiv) a heavy chain variable region comprising an amino acid sequence having at least 90% identity to SEQ ID NO: 137 and a light chain variable region comprising an amino acid sequence having at least 90% identity to SEQ ID NO: 314; (xxv) a heavy chain variable region comprising an amino acid sequence having at least 90% identity to SEQ ID NO: 149 and a light chain variable region comprising an amino acid sequence having at least 90% identity to SEQ ID NO: 326; (xxvi) a heavy chain variable region comprising an amino acid sequence having at least 90% identity to SEQ ID NO: 156 and a light chain variable region comprising an amino acid sequence having at least 90% identity to SEQ ID NO: 333; (xxvii) a heavy chain variable region comprising an amino acid sequence having at least 90% identity to SEQ ID NO: 158 and a light chain variable region comprising an amino acid sequence having at least 90% identity to SEQ ID NO: 335; or (xxviii) a heavy chain variable region comprising an amino acid sequence having at least 90% identity to SEQ ID NO: 143 and a light chain variable region comprising an amino acid sequence having at least 90% identity to SEQ ID NO: 320.

In some embodiments, provided herein is an antibody or fragment thereof, wherein the antibody comprises. (i) a heavy chain variable region comprising an amino acid sequence according to SEQ ID NO: 114 and a light chain variable region comprising an amino acid sequence according to SEQ ID NO: 291; (ii) a heavy chain variable region comprising an amino acid sequence according to SEQ ID NO: 115 and a light chain variable region comprising an amino acid sequence according to SEQ ID NO: 292; (iii) a heavy chain variable region comprising an amino acid sequence according to SEQ ID NO: 116 and a light chain variable region comprising an amino acid sequence according to SEQ ID NO: 293; (iv) a heavy chain variable region comprising an amino acid sequence according to SEQ ID NO: 117 and a light chain variable region comprising an amino acid sequence according to SEQ ID NO: 294; (v) a heavy chain variable region comprising an amino acid sequence according to SEQ ID NO: 118 and a light chain variable region comprising an amino acid sequence according to SEQ ID NO: 295; (vi) a heavy chain variable region comprising an amino acid sequence according to SEQ ID NO: 119 and a light chain variable region comprising an amino acid sequence according to SEQ ID NO: 296; (vii) a heavy chain variable region comprising an amino acid sequence according to SEQ ID NO: 120 and a light chain variable region comprising an amino acid sequence according to SEQ ID NO: 297; (viii) a heavy chain variable region comprising an amino acid sequence according to SEQ ID NO: 121 and a light chain variable region comprising an amino acid sequence according to SEQ ID NO: 298; (ix) a heavy chain variable region comprising an amino acid sequence according to SEQ ID NO: 122 and a light chain variable region comprising an amino acid sequence according to SEQ ID NO: 299; (x) a heavy chain variable region comprising an amino acid sequence according to SEQ ID NO: 123 and a light chain variable region comprising an amino acid sequence according to SEQ ID NO: 300. (xi) a heavy chain variable region comprising an amino acid sequence according to SEQ ID NO: 124 and a light chain variable region comprising an amino acid sequence according to SEQ ID NO: 301; (xii) a heavy chain variable region comprising an amino acid sequence according to SEQ ID NO: 125 and a light chain variable region comprising an amino acid sequence according to SEQ ID NO: 302; (xiii) a heavy chain variable region comprising an amino acid sequence according to SEQ ID NO: 126 and a light chain variable region comprising an amino acid sequence according to SEQ ID NO: 303; (xiv) a heavy chain variable region comprising an amino acid sequence according to SEQ ID NO: 127 and a light chain variable region comprising an amino acid sequence according to SEQ ID NO: 304; (xv) a heavy chain variable region comprising an amino acid sequence according to SEQ ID NO: 128 and a light chain variable region comprising an amino acid sequence according to SEQ ID NO: 305; (xvi) a heavy chain variable region comprising an amino acid sequence according to SEQ ID NO: 129 and a light chain variable region comprising an amino acid sequence according to SEQ ID NO: 306; (xvii) a heavy chain variable region comprising an amino acid sequence according to SEQ ID NO: 130 and a light chain variable region comprising an amino acid sequence according to SEQ ID NO: 307; (xviii) a heavy chain variable region comprising an amino acid sequence according to SEQ ID NO: 131 and a light chain variable region comprising an amino acid sequence according to SEQ ID NO: 308; (xix) a heavy chain variable region comprising an amino acid sequence according to SEQ ID NO: 132 and a light chain variable region comprising an amino acid sequence according to SEQ ID NO: 309; (xx) a heavy chain variable region comprising an amino acid sequence according to SEQ ID NO: 133 and a light chain variable region comprising an amino acid sequence according to SEQ ID NO: 310; (xxi) a heavy chain variable region comprising an amino acid sequence according to SEQ ID NO: 134 and a light chain variable region comprising an amino acid sequence according to SEQ ID NO: 311; (xxii) a heavy chain variable region comprising an amino acid sequence according to SEQ ID NO: 135 and a light chain variable region comprising an amino acid sequence according to SEQ ID NO: 312; (xxiii) a heavy chain variable region comprising an amino acid sequence according to SEQ ID NO: 136 and a light chain variable region comprising an amino acid sequence according to SEQ ID NO: 313; (xxiv) a heavy chain variable region comprising an amino acid sequence according to SEQ ID NO: 137 and a light chain variable region comprising an amino acid sequence according to SEQ ID NO: 314; (xxv) a heavy chain variable region comprising an amino acid sequence according to SEQ ID NO: 149 and a light chain variable region comprising an amino acid sequence according to SEQ ID NO: 326; (xxvi) a heavy chain variable region comprising an amino acid sequence according to SEQ ID NO: 156 and a light chain variable region comprising an amino acid sequence according to SEQ ID NO: 333; (xxvii) a heavy chain variable region comprising an amino acid sequence according to SEQ ID NO: 158 and a light chain variable region comprising an amino acid sequence according to SEQ ID NO: 335; or (xxviii) a heavy chain variable region comprising an amino acid sequence according to SEQ ID NO: 143 and a light chain variable region comprising an amino acid sequence according to SEQ ID NO: 320.

In some embodiments, provided herein is an antibody or fragment thereof, wherein the antibody or fragment thereof is a monoclonal antibody, a Fab, F(ab′)₂, Fab′, scFv, or a single domain antibody (sdAb).

In some embodiments, provided herein is an antibody or fragment thereof, wherein the antibody comprises a human IgG1 or IgG4 domain.

In some embodiments, provided herein is an antibody or fragment thereof comprising an IgG4 domain having a constant heavy domain according to SEQ ID NO: 1441 and a constant light domain according to SEQ ID NO: 1442.

In some embodiments, provided herein is an antibody or fragment thereof comprising an IgG4 domain comprising a S241P mutation at amino acid residue 241 and an L248E mutation at amino acid residue 248, wherein the numbering of the residues is that of the Kabat numbering system.

In some embodiments, provided herein is an antibody or fragment thereof comprising an IgG4 domain having a constant heavy domain according to SEQ ID NO: 1440 and a constant light domain according to SEQ ID NO: 1442.

In some embodiments, provided herein is an antibody or fragment thereof, comprising a heavy chain amino acid sequence of any one of SEQ ID NOs: 892-914, 926, 933, 935, and 920 and a light chain amino acid sequence of any one of SEQ ID NOs: 1029-1051, 1063, 1070, 1072, and 1057.

In some embodiments, provided herein is an antibody or fragment thereof, comprising a heavy chain amino acid sequence of any one of SEQ ID NOs: 618-640, 652, 659, 661, and 646 and a light chain amino acid sequence of any one of SEQ LD NOs: 755-777, 789, 796, 798, and 783.

In some embodiments, provided herein is an antibody or fragment thereof, comprising an amino acid sequence of any one of SEQ ID NOs: 481-503 and 515, 522, 524, and 509.

In some embodiments, provided herein is an antibody or fragment thereof having a binding affinity for SCF of 50 nM or less.

In some embodiments, provided herein is an antibody or fragment thereof having a binding affinity for SCF of 10 nM or less.

In some embodiments, provided herein is an antibody or fragment thereof having a binding affinity for SCF of 5 nM or less.

In some embodiments, provided herein is an antibody or fragment thereof that blocks the interaction between SCF and c-Kit.

In some embodiments, provided herein is an antibody or fragment thereof that causes internalization of SCF.

In some embodiments, provided herein is an antibody or fragment thereof that specifically binds to SCF248.

In some embodiments, provided herein is an antibody or fragment thereof that does not bind to SCF220.

In some embodiments, provided herein is an isolated nucleic acid molecule encoding any one of the antibodies or fragments thereof described herein.

In some embodiments, provided herein is an expression vector comprising a nucleic acid segment encoding an antibody or fragment thereof described herein. In some embodiments, provided herein is an expression vector comprising a nucleic acid encoding an antibody or fragment thereof described herein.

In some embodiments, provided herein is a recombinant host cell comprising an expression vector comprising a nucleic acid segment encoding an antibody or fragment thereof described herein. In some embodiments, provided herein is a recombinant host cell comprising an expression vector comprising a nucleic acid encoding an antibody or fragment thereof described herein.

In some embodiments, provided herein is a method for inhibiting inflammation or fibrosis in a subject in need thereof, the method comprising administering to the subject an antibody or fragment thereof provided herein.

In some embodiments, provided herein is a method for treating a chronic inflammatory disease or a fibrotic disease in a subject in need thereof, the method comprising administering to the subject an antibody or fragment thereof provided herein.

In some embodiments, provided herein is a method for treating a chronic inflammatory disease or a fibrotic disease in a subject in need thereof, the method comprising administering to the subject an antibody or fragment thereof provided herein, wherein the chronic inflammatory disease or fibrotic disease is selected from the group consisting of urticaria, atopic dermatitis, non-alcoholic steatohepatitis (NASH), primary sclerosing cholangitis, pulmonary fibrosis, chronic obstructive pulmonary disease (COPD), acute respiratory distress syndrome (ARDS), cystic fibrosis, peribronchial fibrosis, hypersentitivity pneumonitis, asthma, bleomycin lung, scleroderma, liver cirrhosis, endomyocardial fibrosis, fibromyalgia, eosinophilic esophagitis, inflammatory bowel disease (IBD), chronic kidney disease (CKD), end stage renal disease (ERSD), renal fibrosis, glomerulonephritis, and nephropathy.

### BRIEF DESCRIPTION OF THE FIGURES

FIG. 1 shows flow cytometry plots of a monoclonal yeast population displaying the parental 5H10 humanized antibody, referred to as “VK3/VH1” as a single chain variable fragment (scFv) on the surface and incubated with increasing concentrations of the target peptide PE9413. PE9413 is a C-terminal biotinylated peptide mapping onto exon 6 of stem cell factor 248 isoform (SCF248). PE9413 has the sequence of SEQ ID NO: 479. The x-axis shows display of the scFv on the surface of yeast cells, and the y-axis shows binding to PE9413.

FIG. 2 is a plot of concentration of PE9413 (labeled “target concentration”) versus the median fluorescent signal of the yeast cell population bound to PE9413. The yeast cell population displayed the parental 5H10 humanized antibody as an scFv on its surface. The curve was used to determine that the affinity of the parental 5H10 humanized scFv for C-terminal biotinylated PE9413 was 173.8 nM (R²=0.9922).

FIG. 3 shows PCR assembly of the 6 mutational scanning libraries. In each library, one of the CDRs is replaced by a synthetic oligonucleotide carrying a single mutation at an amino acid position of a CDR.

FIG. 4 shows flow cytometry plots of yeast cells displaying 5H10 VK3/VH1 mutational scanning CDR libraries stained with 170 nM of the PE9413 target peptide or in the absence of PE9413 target peptide (“0 nM PE9413”). The yeast cells displaying scFv with binding signal above background (square) were sorted for additional rounds of selections. The binding profile of the VK3/VH1 stained at the same target antigen concentration is reported on the right panel.

FIG. 5 shows flow cytometry plots of yeast cells displaying 5H10 VK3/VH1 mutational scanning CDR libraries stained with 85 nM of the PE9413 peptide. Each member of the library contains one mutated CDR (e.g., HCDR1, HCDR2, HCDR3, LCDR1, LCDR2, or LCDR3).

FIG. 6 shows a WebLogo representation of the mutations in each 5H10 VK3/VH1 mutational scanning CDR library after 2 rounds of sorting. Eight random clones from each selected library were Sanger sequenced and used in the WebLogo analysis. The height of the letter in the representation represents the frequency of an amino acid occurring at a particular CDR position.

FIG. 7 is a graphical representation of combinatorial library assembly from the individual CDR selected libraries.

FIG. 8A shows flow cytometry plots of yeast displaying the combinatorial library of Example 2. The yeast were stained with PE9413 and a control peptide (labeled Ctrl peptide), having a sequence according to SDGKSPNSDNSPSRKSLSASR (SEQ ID NO: 480) at 10 nM, 25 nM, and 85 nM.

FIG. 8B shows flow cytometry plots of yeast displaying the combinatorial library of Example 2. The yeast were stained with PE9413 and a control peptide (labeled Ctrl peptide), having a sequence according to SEQ ID NO: 480, at 10 nM, 25 nM, 50 nM, 85 nM, and 170 nM.

FIG. 9A shows binding of the magnetic-activated cells sorted (MACS) yeast cells to PE9413 (1 nM, 5 nM, 10 nM, 25 nM).

FIG. 9B shows the gating strategy for fluorescence-activated cell sorting (FACS) of a yeast combinatorial library. The top 1% of yeast cells that bound to 9413 (indicated by a box) were selected for.

FIG. 9C shows binding of the FACS selected yeast cells to PE9413 at 0 nM, 5 nM, or 10 nM PE9413.

FIG. 10A shows binding of the combinatorial library or the parental scFv to biotinylated PE9413 after competition with unlabeled PE9413. The library was incubated with 5 nM of PE9413.

FIG. 10B shows binding of the combinatorial library or the parental scFv to biotinylated PE9413 after competition with unlabeled PE9413. The library is saturated with biotinylated PE9413.

FIG. 10C shows the population of yeast cells (indicated by a box) that are selected after the kinetic sort.

FIG. 11 shows a WebLogo representation of the CDRs of affinity matured clones. The height of the letter in the representation represents the frequency of an amino acid occurring at a particular CDR position.

FIG. 12 shows the affinity of the selected clones for N-terminal biotinylated PE9413 (labeled “N-biot”) versus C-terminal biotinylated PE9413 (labeled “C-biot”).

FIG. 13 is a graphic that shows how yeast display is used for antibody discovery.

FIG. 14 shows binding of yeast cells sorted by MACS to PE9413.

FIG. 15 is a cartoon representation of the steps of the kinetic sort described in Example 2.

FIG. 16A shows binding of the selected kinetically sorted yeast cells to 0 nM, 1 nM, 5 nm, 10 nM, 15 nM, 25 nM, and 50 nM PE9413.

FIG. 16B shows binding of the selected kinetically sorted yeast cells to 85 nM, 170 nM, 200 nM, 400 nM, and 2000 nM PE9413.

FIG. 16C is a plot of concentration of PE9413 (labeled “concentration”) versus the median fluorescent signal of the kinetically sorted yeast combinatorial library that bound to PE9413. The curve was used to determine that the affinity of the combinatorial library for C-terminal biotinylated PE9413 was 6.4 nM (R²=0.9964).

FIG. 16D is a plot of concentration of PE9413 (labeled “concentration”) versus the median fluorescent signal of yeast cells displaying the parental 5H10 scFv. The curve was used to determine that the affinity of the parental 5H10 scFv for C-terminal biotinylated PE9413 was 232 nM (R²=0.9922).

FIG. 17 shows flow cytometry plots of a combinatorial yeast library after a final equilibrium sort. The x-axis shows display of the scFv, and the y-axis shows binding of each clone to PE9413.

FIG. 18 provides a schematic overview of the tissue injury/inflammatory disease process.

FIG. 19 shows an exemplary mechanism of an anti-SCF248 antibody of the instant disclosure, 5H10. The 5110 antibody is referred to in the figure as “anti-SCF248”.

FIG. 20 shows the isoforms of SCF, SCF220, SCF 248, and the monomeric cleaved extracellular domain, SCF165. SCF165 is released upon cleavage of SCF248 at its cleavage site within the Exon 6 region.

FIG. 21 shows the binding of each variant of 5H10 as a function of variant concentration.

FIG. 22 shows the effect of each variant of 5H10 on CCL2 mRNA expression relative to a positive control.

FIG. 23 shows the effect of each variant of 5H10 on TGFβ mRNA expression relative to a positive control.

### DETAILED DESCRIPTION

Stem Cell Factor (SCF) is a key mediator of acute and chronic inflammation, fibrotic diseases, and tissue remodeling diseases. The interaction of SCF with c-Kit on immune cells initiates and perpetuates inflammation and fibrosis. The present disclosure provides compositions and methods for inhibiting the interaction of SCF with c-Kit. In one aspect, the present disclosure provides compositions and methods for preventing the inflammatory form of SCF, SCF248, from interacting with c-Kit and thus reduces and/or prevents activation of immune cells. In aspects, the compositions provided herein are antibodies against SCF that have high binding affinity for SCF. Thus, the present disclosure provides methods for treating chronic inflammation and fibrotic and tissue remodeling diseases, the methods comprising administering to subjects in need thereof an antibody with high affinity to SCF. In one aspect, the present disclosure provides compositions and methods for reducing the accumulation (e.g., proliferation and/or retention) of immune cells in an organ or tissue. For example, the disclosure provides compositions and methods that prevent SCF248 from interacting with c-Kit and thus reduces and/or prevents accumulation of immune cells in organs or tissues. In some embodiments, the disclosure provides compositions and methods for reducing and/or preventing the activation and/or accumulation in organs or tissues of mast cells, eosinophils, type 2 innate lymphoid (ILC2) cells, and type 3 innate lymphoid (ILC3) cells.

In particular, the present disclosure provides antibodies and antigen-binding fragments thereof that specifically bind to SCF and block or inhibit its interaction with c-Kit. In embodiments, the antibodies have high affinity for SCF, for example, affinity in the range of about 1 nM to about 20 nM, about 1 nM to about 10 nM, about 1 nM to about 5 nM, or about 1 nM to about 4 nM, about 2 nM to about 5 nM, about 2 nM to about 4 nM, about 2 nM to about 20 nM, or about 2 nM to about 10 nM. In some embodiments, the antibodies and fragments thereof provided herein bind to SCF and inhibit the activity of c-Kit and c-Kit+ cells. The disclosure also provides diagnostic methods of use of the antibodies provided herein. In one aspect, the antibodies and fragments thereof provided herein specifically bind to the SCF isoform that drives inflammation, SCF248, with high affinity. Thus, the present disclosure provides specific, effective compositions and methods for inhibiting inflammation and fibrosis and treating chronic inflammatory diseases and fibrotic diseases.

### Definitions

As used herein, the term “antibody” refers to a binding protein having at least one antigen binding domain. The antibodies and fragments thereof of the present invention may be whole antibodies or any fragment thereof. Thus, the antibodies and fragments of the invention include monoclonal antibodies or fragments thereof and antibody variants or fragments thereof, as well as immunoconjugates. Antigen binding fragments include Fab fragments, Fab′ fragments, F(ab′)₂ fragments, bispecific Fab dimers (Fab2), trispecific Fab trimers (Fab3), Fv, single chain Fv proteins (“scFv”), bis-scFv, (scFv)2, minibodies, diabodies, triabodies, tetrabodies, disulfide stabilized Fv proteins (“dsFv”), single-domain antibodies (sdAb, nanobody), heavy-chain only antibodies (e.g., camelid VHH, camelid nanobody, shark Ig NAR), and portions of full length antibodies responsible for antigen binding. An isolated antibody or antigen binding fragment thereof is one which has been identified and separated and/or recovered from a component of its natural environment.

In some embodiments, the antibodies and antigen binding fragments thereof are isolated antibodies and fragments thereof, Thus, the present invention provides isolated antibodies and antigen binding fragments thereof, and nucleic acids encoding such antibodies and fragments, as well as compositions comprising such isolated antibodies, fragments, and nucleic acids. The term “isolated” refers to a compound of interest (e.g., an antibody or nucleic acid) that has been separated from its natural environment. The present invention further provides pharmaceutical compositions comprising the isolated antibodies or fragments thereof, or nucleic acids encoding such antibodies or fragments, and further comprising one or more pharmaceutically acceptable carrier. Pharmaceutically acceptable carriers include, for example, excipients, diluents, encapsulating materials, fillers, buffers, or other agents.

As used herein, the term “derived” when used to refer to a molecule or polypeptide relative to a reference antibody or other binding protein, means a molecule or polypeptide that is specific for, and capable of binding to, the same epitope as the reference antibody or other binding protein.

As used herein, the phrase “specific for” may mean that the antibody does not bind to the target due to only non-specific interactions, and this property can be determined by comparison to an isotype control or similar. Specific binding does not necessarily require, although it may include, exclusive binding to a single target. In embodiments, the antibodies provided herein specifically bind to SCF248, and do not bind SCF220. In embodiments, the antibodies provided herein specifically bind to SCF248 with an affinity (K_{D}) of about 20 nM or lower, and do not bind to SCF220.

The term “host cell” means a cell that has been transformed, or is capable of being transformed, with a nucleic acid sequence and thereby expresses a gene of interest. The term includes the progeny of the parent cell, whether or not the progeny is identical in morphology or in genetic make-up to the original parent cell, so long as the gene of interest is present.

A “variant” of a polypeptide (e.g., an antigen binding protein, or an antibody) comprises an amino acid sequence wherein one or more amino acid residues are inserted into, deleted from and/or substituted into the amino acid sequence relative to another polypeptide sequence. Variants include antibodies and fragments thereof that have a recited percent identity to an antibody or fragment provided herein or to an antibody or fragment having a recited DNA or amino acid sequence.

The term “identity” refers to a relationship between the sequences of two or more polypeptide molecules or two or more nucleic acid molecules, as determined by aligning and comparing the sequences. “Percent identity,” “percent homology,” “sequence identity,” or “sequence homology” and the like mean the percent of identical residues between the amino acids or nucleotides in the compared molecules and is calculated based on the size of the smallest of the molecules being compared. For example, the terms percent homology, sequence identity, sequence homology, and the like refer to the number of identical amino acid sequences shared by two reference sequences, divided by the total number of amino acid positions, multiplied by 100. For these calculations, gaps in alignments (if any) are preferably addressed by a particular mathematical model or computer program (i.e., an “algorithm”). Methods that can be used to calculate the identity of the aligned nucleic acids or polypeptides include those described in Computational Molecular Biology, (Lesk, A. M., ed.), 1988, New York: Oxford University Press; Biocomputing Informatics and Genome Projects, (Smith, D. W., ed.), 1993, New York: Academic Press; Computer Analysis of Sequence Data, Part I, (Griffin, A. M., and Griffin, H. G., eds.), 1994, New Jersey: Humana Press; von Heinje, G., 1987, Sequence Analysis in Molecular Biology, New York: Academic Press: Sequence Analysis Primer, (Gribskov, M. and Devereux, J., eds.), 1991, New York: M. Stockton Press; and Carillo et al., 1988, SIAM J. Applied Math. 48:1073. In calculating percent identity, the sequences being compared are typically aligned in away that gives the largest match between the sequences.

The term “light chain” includes a full-length light chain and fragments thereof having sufficient variable region sequence to confer binding specificity. A full-length light chain includes a variable region domain and a constant region domain. The variable region domain of the light chain is at the amino-terminus of the polypeptide. Light chains include kappa chains and lambda chains.

The term “heavy chain” includes a full-length heavy chain and fragments thereof having sufficient variable region sequence to confer binding specificity. A full-length heavy chain includes a variable region domain, three constant region domains, C_{H}1, C_{H}2, and C_{H}3. The variable heavy domain is at the amino-terminus of the polypeptide, and the C_{H} domains are at the carboxyl-terminus, with the C_{H}3 being closest to the carboxy-terminus of the polypeptide. Heavy chains can be of any isotype, including IgG (including IgG1, IgG2, IgG3 and IgG4 subtypes), IgA (including IgA1 and IgA2 subtypes), IgM and IgE. The term “isotype” refers to the antibody class encoded by the heavy chain constant region genes. In some embodiments, the antibodies provided herein have an IgG4 heavy chain, or an IgG4 heavy chain comprising certain amino acid mutations. For example, in some embodiments, the IgG4 comprises a mutation at position 228 (EU numbering scheme, Kabat et al. Sequence of proteins of immunologic interest, 5th ed Bethesda, MD, NIH 1991) to inhibit Fab arm exchange. For example, in some embodiments, the IgG4 heavy chain is an IgG4 S228P heavy chain. In some embodiments, the heavy chain comprises one or more amino acid mutations that reduce binding to Fc receptors, and thereby reduce or eliminate effector function of the antibody. For example, the heavy chain may comprise mutations at one or more of positions 233, 234, 235, 236, 237, 241, 265, 309, 331, and 409 (EU numbering). In some embodiments, the IgG4 heavy chain comprises a mutation at position 241. In some embodiments, position 241 is mutated to proline.

The term “variable region” or “variable domain” refers to a portion of the light and/or heavy chains of an antibody, typically including approximately the amino-terminal 120 to 130 amino acids in the heavy chain and about 100 to 110 amino terminal amino acids in the light chain. Light chain variable regions may be referred to herein as “VL” or “VI”. Heavy chain variable regions may be referred to herein as “VH” or “Vh”. In certain embodiments, variable regions of different antibodies differ extensively in amino acid sequence even among antibodies of the same species. The variable region of an antibody typically determines specificity of a particular antibody for its target, by way of the complementary determining regions (CDRs) therein. The term “target,” as used herein, refers to a molecule or a portion of a molecule capable of being bound by an antigen binding protein. In certain embodiments, a target can have one or more epitopes. In certain embodiments, a target is an antigen. The use of “antigen” in the phrase “antigen binding protein” simply denotes that the protein sequence that comprises the antigen can be bound by an antibody. In this context, it does not require that the protein be foreign or that it be capable of inducing an immune response.

The term “epitope” includes any determinant capable being bound by an antigen binding protein, such as an antibody or to a T-cell receptor. An epitope is a region of an antigen that is bound by an antigen binding protein that targets that antigen, and when the antigen is a protein, includes specific amino acids that directly contact the antigen binding protein. Most often, epitopes reside on proteins, but in some instances can reside on other kinds of molecules, such as nucleic acids. Epitope determinants can include chemically active surface groupings of molecules such as amino acids, sugar side chains, phosphoryl or sulfonyl groups, and can have specific three dimensional structural characteristics, and/or specific charge characteristics. Generally, antibodies specific for a particular target antigen will preferentially recognize an epitope on the target antigen in a complex mixture of proteins and/or macromolecules. Antibody epitopes may be linear or conformational. In embodiments, the epitope provided herein is a linear epitope.

The use of the singular includes the plural unless specifically stated otherwise. The word “a” or “an” means “at least one” unless specifically stated otherwise. The use of “or” means “and/or” unless stated otherwise. The meaning of the phrase “at least one” is equivalent to the meaning of the phrase “one or more.” Furthermore, the use of the term “including,” as well as other forms, such as “includes” and “included,” is not limiting. Also, terms such as “element” or “component” encompass both elements or components comprising one unit and elements or components comprising more than one unit unless specifically stated otherwise. As used herein, the term “about” refers to an amount more or less than the stated parameter value, for example plus or minus five or ten percent of the object that “about” modifies, or as one of skill in the art would recognize from the context (e.g., approximately 50% of the interval between values). The term “about” also includes the value referenced.

### Stem Cell Factor

In humans, there are at least two forms of SCF, which have different structures and activities. SCF220 functions in several homeostatic functions, including hematopoiesis and spermatogenesis and is found in bone marrow, testis, and other tissues and organs. SCF220 is slowly cleavable and sometimes called “membrane SCF.” In contrast, SCF248 is rapidly cleavable and comprises a cleavage site in exon 6, located between the N-terminal c-kit binding domain and the transmembrane domain. SCF248 may be referred to as “soluble SCF”. Exon 6 is excluded from SCF220 via alternative splicing, and SCF220 thus lacks this cleavage site. A monomeric, extracellular domain (SCF165) is the cleavage product and serves as a biomarker in plasma for chronic inflammatory diseases. Plasma may also contain detectable levels of SCF extracellular domain that comes from SCF220, but the majority of detectable extracellular domain is expected to be SCF165. SCF248 is the isoform found on myofibroblasts, activated epithelial cells, and other cells, which activates immune cells during inflammation and contributes to perpetuation of fibrosis. More specifically, SCF248 binds to c-Kit on immune cells, initiating production of cytokines that activate fibroblasts to become myofibroblasts, which secrete extracellular matrix proteins, collagen, and fibronectin. The activated myofibroblasts as well as activated epithelia, endothelia, macrophages, eosinophils, mast cells, monocytes, and other cells also express SCF on the cell surface, activating more c-Kit+ immune cells, resulting in further cytokine release and immune activation and fibrotic responses.

The antibodies and antigen-binding fragments thereof disclosed herein are specific for SCF. In some embodiments, the antibodies and fragments thereof are specific for human SCF. In some embodiments, the antibodies and fragments thereof are specific for SCF248. In some embodiments, the antibodies bind SCF248 and do not bind other isoforms of SCF. In some embodiments, the antibodies bind SCF248 and do not bind to SCF220. In some embodiments, the present disclosure provides methods for making an antibody or fragment thereof that is specific for SCF248. Exemplary antibodies and fragments that are specific for SCF248, as well as methods for making and using the antibodies and fragments, are provided in the present disclosure. In some embodiments, the antibodies and fragments thereof provided herein breaks the positive feedback loop between SCF248 expressed on various cell types and cKit+ immune cells, by binding to SCF248 and blocking the interaction between SCF248 and c-Kit.

### Antibodies and Fragments

The present disclosure provides antibodies, including monoclonal antibodies, and fragments thereof. The antibody fragments provided herein that are specific for SCF (e.g., SCF248) are sometimes referred to herein as antigen-binding fragments, meaning that they comprise the portion of the parent antibody that is capable of binding the target antigen (SCF, e.g., SCF248). “Antibody fragment,” “antigen binding fragment” and the like are used interchangeably herein. Examples of antibody fragments include Fab fragments, Fab′ fragments, F(ab)′ fragments, Fv fragments, isolated CDR regions, bispecific Fab dimers (Fab2), trispecific Fab trimers (Fab3), single chain Fv proteins (“scFv”), bis-scFv, (scFv)2, minibodies, diabodies, triabodies, tetrabodies, disulfide stabilized Fv proteins (“dsFv”), single-domain antibodies (sdAb, nanobody), heavy-chain only antibodies (e.g., camelid VHH, camelid nanobody, shark Ig NAR), and portions of full length antibodies responsible for antigen binding.

A “Fab fragment” comprises one light chain and the C_{H}1 and variable regions of one heavy chain. The heavy chain of a Fab molecule cannot form a disulfide bond with another heavy chain molecule. A “Fab′ fragment” comprises one light chain and a portion of one heavy chain that contains the VH domain and the C_{H}1 domain and also the region between the C_{H}1 and C_{H}2 domains, such that an interchain disulfide bond can be formed between the two heavy chains of two Fab′ fragments to form an F(ab′)₂ molecule. A “F(ab′)₂ fragment” contains two light chains and two heavy chains containing a portion of the constant region between the CI and C_{H}2 domains, such that an interchain disulfide bond is formed between the two heavy chains A F(ab′)₂ fragment thus is composed of two Fab′ fragments that are held together by a disulfide bond between the two heavy chains. A “Fv fragment” comprises the variable regions from both the heavy and light chains, but lacks the constant regions. “scFvs” are Fv molecules in which the heavy and light chain variable regions have been connected by a flexible linker to form a single polypeptide chain, which forms an antigen binding region.

In some aspects, the antibodies and fragments thereof provided herein are defined by their complementary determining regions (CDRs). CDRs are part of the variable chains in antibodies; each of the light and heavy chain variable regions comprises three CDRs, CDR1, CDR2, and CDR3. The CDRs of an antibody determine antigen specificity. In certain embodiments, definitive delineation of a CDR and identification of residues comprising the binding site of an antibody is accomplished by solving the structure of the antibody and/or solving the structure of the antibody-ligand complex. In certain embodiments, that can be accomplished by any of a variety of techniques known to those skilled in the art, such as X-ray crystallography. In certain embodiments, various methods of analysis can be employed to identify or approximate the CDR regions. Examples of such methods include, but are not limited to, the Kabat definition, the Chothia definition, the AbM definition and the contact definition.

The Kabat definition is a standard for numbering the residues in an antibody and is typically used to identify CDR regions. See, e.g., Johnson & Wu, Nucleic Acids Res., 28: 214-8 (2000). The Chothia definition is similar to the Kabat definition, but the Chothia definition takes into account positions of certain structural loop regions. See, e.g., Chothia et al., J. Mol. Biol., 196: 901-17 (1986); Chothia et al., Nature, 342: 877-83 (1989). The AbM definition uses an integrated suite of computer programs produced by Oxford Molecular Group that model antibody structure. See, e.g., Martin et al., Proc Natl Acad Sci (USA), 86:9268-9272 (1989); “AbM™, A Computer Program for Modeling Variable Regions of Antibodies,” Oxford, UK; Oxford Molecular, Ltd. The AbM definition models the tertiary structure of an antibody from primary sequence using a combination of knowledge databases and ab initio methods, such as those described by Samudrala et al., “Ab Initio Protein Structure Prediction Using a Combined Hierarchical Approach,” in PROTEINS, Structure, Function and Genetics Suppl., 3:194-198 (1999). The contact definition is based on an analysis of the available complex crystal structures. See, e.g., MacCallum et al., J. Mol. Biol., 5:732-45 (1996).

Antibodies and fragments thereof may also include recombinant polypeptides, fusion proteins, and bi-specific antibodies. The anti-SCF antibodies and fragments thereof disclosed herein may be of an IgG1, IgG2, IgG3, or IgG4 isotype. In one embodiment, the anti-SCF antibodies and fragments thereof disclosed herein are of an IgG1 or an IgG4 isotype. The anti-SCF antibodies and fragments thereof of the present invention may be derived from any species including, but not limited to, mouse, rat, rabbit, primate, llama, camel, goat, shark, chicken, and human. The SCF antibodies and fragments thereof may be chimeric, humanized, or fully human antibodies. In one embodiment, the anti-SCF antibodies are murine antibodies. In another embodiment, the anti-SCF antibodies are chimeric antibodies. In a further embodiment, the chimeric antibodies are mouse-human chimeric antibodies. In another embodiment, the antibodies are derived from mice and are humanized.

A “chimeric antibody” is an antibody having at least a portion of the heavy chain variable region and at least a portion of the light chain variable region derived from one species; and at least a portion of a constant region derived from another species. For example, in one embodiment, a chimeric antibody may comprise murine variable regions and a human constant region.

A “humanized antibody” is an antibody containing framework regions as well as constant regions that are derived from a human antibody, and complementarity determining regions (CDRs) that were not derived from a human antibody (e.g., were derived from a mouse antibody). In embodiments, the humanized antibodies provided herein bind to the same epitope on SCF as a murine antibody from which the antibody's CDRs are derived. In embodiments, the antibodies provided herein have been generated from a humanized antibody, but have been further modified in one or more CDR regions such that one or more CDRs no longer is identical to the CDRs from the parental murine antibody. In embodiments, the CDR modifications surprisingly enhance affinity for SCF. Generally when humanized antibodies are generated from non-human parental antibodies, the humanized antibodies have equivalent or reduced affinity for the antibody's target antigen, relative to the affinity exhibited by the non-human parental antibody. Surprisingly, antibodies provided herein exhibit significantly enhanced affinity for SCF compared to the parental murine antibody, or compared to the humanized parental antibody from which they were derived.

In some embodiments, the antibodies and fragments thereof provided herein comprise a heavy and light chain, each of which comprises three CDRs. The amino acid sequences of exemplary heavy chain CDR1, CDR2, and CDR3 (HCDR1, HCDR2, and HCDR3, respectively) and light chain CDR1, CDR2, and CDR3 (LCDR1, LCDR2, and LCDR3, respectively) are provided below in Table 1. Table 2 provides the amino acid sequences of exemplary heavy and light chain variable regions. Table 3 provides the amino acid sequences of exemplary scFvs. Table 4 provides the amino acid sequences of exemplary antibodies. Some antibodies comprise a human IgG4 domain comprising a S241P mutation at amino acid residue 241 and an L248E mutation at amino acid residue 248, wherein the numbering of the residues is that of the Kabat numbering system, wherein the constant heavy domain has an amino acid sequence of SEQ ID NO: 1440 and a constant light domain has an amino acid sequence of SEQ ID NO: 1442. Other exemplary antibodies have a constant heavy domain of SEQ ID NO: 1441 and a constant light domain according to SEQ ID NO: 1442. In some embodiments, the antibodies provided herein were derived from a humanized parental antibody referred to herein as “5H10 VK3/VH1.” This humanized parental antibody was derived from murine antibody 5H10.

TABLE 1
Exemplary anti-SCF antibody CDR and VH/VL sequences
(parentals + 27 unique clones)

| | HCDR1 | HCDR1 | | HCDR2 | HCDR3 | HCDR3 | | LCDR1 | LCDR2 | LCDR2 | LCDR3 | LCDR3 |
|---|---|---|---|---|---|---|---|---|---|---|---|---|
| | AA | SEQ ID | HCDR2 | SEQ ID | AA | SEQ ID | LCDR1 | SEQ ID | AA | AA | AA | SEQ ID |
| | Seq | NO | AA Seq | NO | Seq | NO | AA Seq | No | Seq | Seq | Seq | NO |
| Parental | SYW | 14 | QIYPGDGDTHY | 15 | SNWV | 16 | KSSQSLLESD | 17 | LVSR | 18 | WQGTH | 19 |
| 5H10 | MN | | NGKFKG | | GSY | | GKTYLN | | LDS | | LPQT | |
| (murine) | | | | | | | | | | | | |
| Parental | SYW | 14 | QIYPGDGDTHY | 15 | SNWV | 16 | KSSQSLLESD | 17 | LVSR | 18 | WQGTH | 19 |
| 5H10 | MN | | NGKFKG | | GSY | | GKTYLN | | LDS | | LPOT | |
| VK3/VH1 | | | | | | | | | | | | |
| (human- | | | | | | | | | | | | |
| ized) | | | | | | | | | | | | |
| A84P | SQW | 22 | QIYPEDGDTHM | 26 | SNWV | 16 | KSSQSLLEED | 71 | LVDR | 90 | WQGTD | 111 |
| | MN | | NGKFKG | | GSY | | GNTYLN | | LDI | | LPOT | |
| A64P | SNW | 23 | QTYPEDGDTHY | 27 | SNWV | 16 | KSSQSLLEED | 71 | LVNR | 91 | WQGID | 111 |
| | MN | | NGKFKG | | GSY | | GNTYLN | | LDD | | LPOT | |
| B114P | SQW | 22 | QIYPEDGDTHM | 26 | SNWD | 67 | KSSQSLLEED | 71 | LVDR | 92 | WQGTD | 111 |
| | MN | | NGKFKG | | GSY | | GNTYLN | | LDS | | LPQT | |
| A104P | SQW | 22 | QIYPEDGDTHY | 27 | SNWV | 16 | KSSQSLLEED | 73 | LVDR | 92 | WQGTD | 111 |
| | MN | | NGKFKG | | GSY | | GNTYLN | | LDS | | LPQT | |
| B124P | SNW | 23 | QIYPEDGDTHY | 28 | SNWV | 16 | KSSQSLLEQD | 72 | LVDR | 92 | WQGTD | 111 |
| | MN | | NDKFKG | | GSY | | GNTYLN | | LDS | | LPQT | |
| H74P | SQW | 22 | QIYPEDGDTHY | 27 | SNWV | 16 | KSSQSLLEED | 71 | LVDR | 93 | WQGSH | 112 |
| | MN | | NGKFKG | | GSY | | GNTYLN | | LDL | | LPQT | |
| H63P | SQW | 22 | QIYPEDGDTHY | 28 | SNWV | 16 | KSSQSLLESD | 73 | LVDR | 92 | WQGTD | 111 |
| | MN | | NDKFKG | | GSY | | GNTYLN | | LDS | | LPQT | |
| H83P | SQW | 22 | QIYPGDGDIHY | 29 | SNWV | 16 | KSSQSLLESD | 73 | LVDR | 92 | WQGTD | 111 |
| | MN | | NDKFKG | | GSY | | GNTYLN | | LDS | | LPOT | |
| F54P | SQW | 22 | QIYPEDGDTHM | 30 | SNWV | 16 | KSSQSLLEED | 71 | LVDR | 92 | WQGTD | 111 |
| | MN | | NGKFKT | | GSY | | GNTYLN | | LDS | | LPQT | |
| D44P | SQW | 22 | QIYPEDGDIHY | 31 | SNWV | 16 | KSSQSLLEED | 71 | LVDR | 92 | WQGSH | 112 |
| | MN | | NGKFKG | | GSY | | GNTYLN | | LDS | | LPQT | |
| D14P | SQW | 22 | QIYPGDDDTHY | 32 | SNWV | 16 | KSSQSLLESD | 73 | LVDR | 93 | WQGID | 111 |
| | MN | | NDKFKG | | GSY | | GNTYLN | | LDL | | LPOT | |
| B104P | SOW | 22 | QIYPDDGDTHY | 33 | SNWV | 16 | KSSQSLLEAD | 74 | LVDR | 92 | WOGTD | 111 |
| | MN | | NDKFKG | | GSY | | GNTYLN | | LDS | | LPQT | |
| B23P | SYY | 24 | QIYPGDGDTHM | 34 | SNWV | 16 | KSSQSLLESD | 73 | LVSR | 94 | WQGTD | 111 |
| | VIN | | NGKFKG | | GSY | | GNTYLN | | RDS | | LPQT | |
| B34P | SNW | 23 | QIYPEDGDTHY | 28 | SNWV | 16 | KSSQSLLESD | 73 | LVSR | 94 | WQGID | 111 |
| | MN | | NDKFKG | | GSY | | GNTYLN | | RDS | | LPQT | |
| B53P | SQW | 22 | QIYPEDGDIHY | 31 | SNWV | 16 | KSSQSLLESD | 73 | LVDR | 92 | WQGTD | 111 |
| | MN | | NGKFKG | | GSY | | GNTYLN | | LDS | | LPQT | |
| C104P | SQW | 22 | QIYPEDGDTHY | 28 | SNWV | 16 | KSSQSLLESD | 73 | LVDR | 92 | WQGTD | 111 |
| | MN | | NDKFKG | | GSY | | GNTYLN | | LDS | | LPQT | |
| D124P | SQW | 22 | QIYPEDGDTHY | 27 | SNWV | 16 | KSSQSLLESD | 73 | LVDR | 92 | WQGID | 111 |
| | MN | | NGKFKG | | GSY | | GNTYLN | | LDS | | LPOT | |
| D74P | SQW | 22 | QIYPGDNDTHY | 35 | SNWV | 16 | KSSQSLLEAD | 74 | LVDR | 92 | WQGTD | 111 |
| | MN | | NDKFKG | | GSY | | GNTYLN | | LDS | | LPQT | |
| E94P | SQW | 22 | QIYPGDGDTHM | 34 | SNWV | 16 | KSSQSLLEED | 71 | LVDR | 92 | WQGTD | 111 |
| | MN | | NGKFKG | | GSY | | GNTYLN | | LDS | | LPQT | |
| F103P | SQW | 22 | QIYPEDGDTHY | 27 | SNWV | 16 | KSSQSLLDSD | 75 | LVDR | 92 | WQGTD | 111 |
| | MN | | NGKFKG | | GSY | | GNTYLN | | LDS | | LPOT | |
| F64P | SYY | 24 | QIYPGDGDTHM | 34 | SNWV | 16 | KSSQSLLESD | 73 | LVDR | 92 | WQGTD | 111 |
| | MN | | NGKFKG | | GSY | | GNTYLN | | LDS | | LPQT | |
| G14P | SQW | 22 | QIYPGDGDTHY | 36 | SNWV | 16 | KSSQSLLEED | 71 | LVDR | 95 | WQGTD | 111 |
| | MN | | NDKFKG | | GSY | | GNTYLN | | LDD | | LPQT | |
| G24P | SNW | 23 | QIYPEDGDTHY | 28 | SNWV | 16 | KSSQSLLESD | 73 | LVDR | 92 | WQGTD | 111 |
| | MN | | NDKFKG | | GSY | | GNTYLN | | LDS | | LPQT | |
| A44P | SQW | 22 | QIYPEDGDTHY | 28 | SNWV | 16 | KSSQSLLESD | 73 | LVNR | 96 | WQGTD | 111 |
| | MN | | NDKFKG | | GSY | | GNTYLN | | LDS | | LPOT | |
| G104P | SQW | 22 | QIYPGDGDTHM | 34 | SNWV | 16 | KSSQSLLEED | 71 | LVDR | 95 | WQGID | 111 |
| | MN | | NGKFKG | | GSY | | GNTYLN | | LDD | | LPQT | |
| D113P | SQW | 22 | QIYPGDGDTHM | 34 | SNWV | 16 | KSSQSLLEGD | 79 | LVDR | 90 | WOGTD | 111 |
| | MN | | INGKFKG | | GSY | | GNTYLN | | LDI | | LPQT | |
| E93P | SOW | 22 | QIYPGDGDTHM | 34 | SNWV | 16 | KSSQSLLDSD | 75 | LVDR. | 92 | WOGTD | 111 |
| | MN | | NGKFKG | | GSY | | GNTYLN | | LDS | | LPQT | |
| D24P | SQW | 22 | QIYPEDGDTHY | 27 | SNWV | 16 | KSSQSLLESD | 73 | LVSR | 98 | WQGID | 111 |
| | MN | | NGKFKG | | GSY | | GNTYLN | | LDI | | LPQT | |

TABLE 2
Exemplary anti-SCF antibody VH/VL sequences

| | | VH | | VL |
|---|---|---|---|---|
| | | SEQ | | SEQ |
| | | ID | | ID |
| Clone | VH | NO | VL | NO |
| Parental | QVQLQQSGAELVRPGSSVKISCKSSGYAFSS | 468 | DVVMTQTPLTLSVTIGQTASISCKSSQSLLESDGK | 469 |
| 5H10 | YWMNWVKQRPGQGLEWIGQIYPGDGDTHYNG | | TYLNWLSQRPGQSPKRLIYLVSRLDSGVPDRFTGS | |
| (murine) | KFKGKATLTADKSSSTAYMQLSRLTSEDSAV | | GSGTDFTLKISRVEAEDLGVYYCWQGTHLPQTFGG | |
| | YFCSSSNWVGSYWGQGTLVTVSA | | GTKLEIK | |
| Parental | QVQLVQSGAELKKPGSSVKISCKSSGYAFSS | 20 | DVVMTQSPLSLPVILGQPASISCKSSQSLLESDGK | 21 |
| 5H10 | YWMNWVKQRPGQGLEWIGQIYPGDGDTHYNG | | TYLNWLQQRPGQSPRRLIYLVSRLDSGVPDRFSGS | |
| VK3/VH1 | KFKGKATLTADKSTSTAYMELSSLTSEDSAV | | GSGTDFTLKISRVEAEDVGVYYCWQGTHLPQTFGG | |
| (human- | YFCSSSNWVGSYWGQGTLVTVSS | | GTKVEIK | |
| ized) | | | | |
| A84P | QVQLVQSGAELKKPGSSVKISCKSSGYAFSS | 114 | DVVMTQSPLSLPVILGQPASISCKSSQSLLEEDGN | 291 |
| | QWMNWVKQRPGQGLEWIGQIYPEDGDTHMNG | | TYLNWLQQRPGQSPRRLIYLVDRLDIGVPDRFSGS | |
| | KFKGKATLTADKSTSTAYMELSSLTSEDSAV | | GSGTDFTLKISRVEAEDVGVYYCWQGIDLPQTFGG | |
| | YFCSSSNWVGSYWGQGTLVTVSS | | GTKVEIK | |
| A64P | QVQLVQSGAELKKPGSSVKISCKSSGYAFSS | 115 | DVVMTQSPLSLPVILGQPASISCKSSQSLLEEDGN | 292 |
| | NWMNWVKQRPGQGLEWIGQIYPEDGDTHYNG | | TYLNWLQQRPGQSPRRLIYLVNRLDDGVPDRFSGS | |
| | KFKGKATLTADKSTSTAYMELSSLTSEDSAV | | GSGTDFTLKISRVEAEDVGVYYCWQGTDLPQTFGG | |
| | YFCSSSNWVGSYWGQGTLVTVSS | | GTKVEIK | |
| B114P | QVQLVQSGAELKKPGSSVKISCKSSGYAFSS | 116 | DVVMTQSPLSLPVILGQPASISCKSSQSLLEEDGN | 293 |
| | QWMNWVKQRPGQGLEWIGQIYPEDGDTHMNG | | TYLNWLQQRPGQSPRRLIYLVDRLDSGVPDRFSGS | |
| | KFKGKATLTADKSTSTAYMELSSLTSEDSAV | | GSGTDFTLKISRVEAEDVGVYYCWQGTDLPQTFGG | |
| | YFCSSSNWDGSYWGQGTLVTVSS | | GTKVEIK | |
| A104P | QVQLVQSGAELKKPGSSVKISCKSSGYAFSS | 117 | DVVMTQSPLSLPVTLGQPASISCKSSQSLLEEDGN | 294 |
| | QWMNWVKQRPGQGLEWIGQIYPEDGDTHYNG | | TYLNWLQQRPGQSPRRLIYLVDRLDSGVPDRFSGS | |
| | KFKGKATLTADKSTSTAYMELSSLTSEDSAV | | GSGTDFTLKISRVEAEDVGVYYCWQGTDLPQTFGG | |
| | YFCSSSNWVGSYWGQGTLVTVSS | | GTKVEIK | |
| B124P | QVQLVQSGAELKKPGSSVKISCKSSGYAFSS | 118 | DVVMTQSPLSLPVILGQPASISCKSSQSLLEQDGN | 295 |
| | NWMNWVKQRPGQGLEWIGQIYPEDGDTHYND | | TYLNWLQQRPGQSPRRLIYLVDRLDSGVPDRFSGS | |
| | KFKGKATLTADKSTSTAYMELSSLTSEDSAV | | GSGTDFTLKISRVEAEDVGVYYCWQGTDLPQTFGG | |
| | YFCSSSNWVGSYWGQGTLVTVSS | | GTKVEIK | |
| H74P | QVQLVQSGAELKKPGSSVKISCKSSGYAFSS | 119 | DVVMTQSPLSLPVTLGQPASISCKSSQSLLEEDGN | 296 |
| | QWMNWVKQRPGQGLEWIGQIYPEDGDTHYNG | | TYLNWLQQRPGQSPRRLIYLVDRLDLGVPDRESGS | |
| | KFKGKATLTADKSTSTAYMELSSLTSEDSAV | | GSGTDFTLKISRVEAEDVGVYYCWQGSHLPQTFGG | |
| | YFCSSSNWVGSYWGQGTLVTVSS | | GTKVEIK | |
| H63P | QVXLVQSGAELKKPGSSVKISCKSSGYAFSS | 120 | DVVMTQSPLSLPVTLGQPASISCKSSQSLLESDGN | 297 |
| | QWMNWVKQRPGQGLEWIGQIYPEDGDTHYND | | TYLNWLQQRPGQSPRRLIYLVDRLDSGVPDRESGS | |
| | KFKGKATLTADKSTSTAYMELSSLTSEDSAV | | GSGTDFTLKISRVEAEDVGVYYCWQGTDLPQTFGG | |
| | YFCSSSNWVGSYWGQGTLVTVSS | | GTKVEIK | |
| H83P | QVQLVQSGAELKKPGSSVKISCKSSGYAFSS | 121 | DVVMTQSPLSLPVILGQPASISCKSSQSLLESDGN | 298 |
| | QWMNWVKQRPGQGLEWIGQIYPGDGDIHYND | | TYLNWLQQRPGQSPRRLIYLVDRLDSGVPDRFSGS | |
| | KFKGKATLTADKSTSTAYMELSSLTSEDSAV | | GSGTDFTLKISRVEAEDVGVYYCWQGTDLPQTFGG | |
| | YFCSSSNWVGSYWGQGTLVTVSS | | GTKVEIK | |
| F54P | QVQLVQSGAELKKPGSSVKISCKSSGYAFSS | 122 | DVVMTQSPLSLPVILGQPASISCKSSQSLLEEDGN | 299 |
| | QWMNWVKQRPGQGLEWIGQIYPEDGDTHMNG | | TYLNWLQQRPGQSPRRLIYLVDRLDSGVPDRFSGS | |
| | KFKTKATLTADKSTSTAYMELSSLTSEDSAV | | GSGTDFTLKISRVEAEDVGVYYCWQGTDLPQTFGG | |
| | YFCSSSNWVGSYWGQGTLVTVSS | | GTKVEIK | |
| D44P | QVQLVQSGAELKKPGSSVKISCKSSGYAFSS | 123 | DVVMTQSPLSLPVILGQPASISCKSSQSLLEEDGN | 300 |
| | QWMNWVKQRPGQGLEWIGQIYPEDGDIHYNG | | TYLNWLQQRPGQSPRRLIYLVDRLDSGVPDRFSGS | |
| | KFKGKATLTADKSTSTAYMELSSLTSEDSAV | | GSGTDFTLKISRVEAEDVGVYYCWQGSHLPQTFGG | |
| | YFCSSSNWVGSYWGQGTLVTVSS | | GTKVEIK | |
| D14P | QVQLVQSGAELKKPGSSVKISCKSSGYAFSS | 124 | DVVMTQSPLSLPVILGQPASISCKSSQSLLESDGN | 301 |
| | QWMNWVKQRPGQGLEWIGQIYPGDDDTHYND | | TYLNWLQQRPGQSPRRLIYLVDRLDLGVPDRESGS | |
| | KFKGKATLTADKSTSTAYMELSSLTSEDSAV | | GSGTDFTLKISRVEAEDVGVYYCWQGTDLPQTFGG | |
| | YFCSSSNWVGSYWGQGTLVTVSS | | GTKVEIK | |
| B104P | QVQLVQSGAELKKPGSSVKISCKSSGYAFSS | 125 | DVVMTQSPLSLPVILGQPASISCKSSQSLLEADGN | 302 |
| | QWMNWVKQRPGQGLEWIGQIYPDDGDTHYND | | TYLNWLQQRPGQSPRRLIYLVDRLDSGVPDRFSGS | |
| | KFKGKATLTADKSTSTAYMELSSLTSEDSAV | | GSGTDFTLKISRVEAEDVGVYYCWQGTDLPQTFGG | |
| | YFCSSSNWVGSYWGQGTLVTVSS | | GTKVEIK | |
| B23P | QVQLVQSGAELKKPGSSVKISCKSSGYAFSS | 126 | DVVMTQSPLSLPVTLGQPASISCKSSQSLLESDGN | 303 |
| | YYMNWVKQRPGQGLEWIGQIYPGDGDTHMNG | | TYLNWLQQRPGQSPRRLIYLVSRRDSGVPDRFSGS | |
| | KFKGKATLTADKSTSTAYMELSSLTSEDSAV | | GSGTDFTLKISRVEAEDVGVYYCWQGTDLPQTFGG | |
| | YFCSSSNWVGSYWGQGTLVTVSS | | GTKVEIK | |
| B34P | QVQLVQSGAELKKPGSSVKISCKSSGYAFSS | 127 | DVVMTQSPLSLPVTLGQPASISCKSSQSLLESDGN | 304 |
| | NWMNWVKQRPGQGLEWIGQIYPEDGDTHYND | | TYLNWLQQRPGQSPRRLIYLVSRRDSGVPDRESGS | |
| | KFKGKATLTADKSTSTAYMELSSLTSEDSAV | | GSGTDFTLKISRVEAEDVGVYYCWQGTDLPQTFGG | |
| | YFCSSSNWVGSYWGQGTLVTVSS | | GTKVEIK | |
| B53P | QVQLVQSGAELKKPGSSVKISCKSSGYAFSS | 128 | DVVMTQSPLSLPVTLGQPASISCKSSQSLLESDGN | 305 |
| | QWMNWVKQRPGQGLEWIGQIYPEDGDIHYNG | | TYLNWLQQRPGQSPRRLIYLVDRLDSGVPDRFSGS | |
| | KFKGKATLTADKSTSTAYMELSSLTSEDSAV | | GSGTDFTLKISRVEAEDVGVYYCWQGTDLPQTFGG | |
| | YFCSSSNWVGSYWGQGTLVTVSS | | GTKVEIK | |
| C104P | QVQLVQSGAELKKPGSSVKISCKSSGYAFSS | 129 | DVVMTQSPLSLPVTLGQPASISCKSSQSLLESDGN | 306 |
| | QWMNWVKQRPGQGLEWIGQIYPEDGDTHYND | | TYLNWLQQRPGQSPRRLIYLVDRLDSGVPDRFSGS | |
| | KFKGKATLTADKSTSTAYMELSSLTSEDSAV | | GSGTDFTLKISRVEAEDVGVYYCWQGTDLPQTFGG | |
| | YFCSSSNWVGSYWGQGTLVTVSS | | GTKVEIK | |
| D124P | QVQLVQSGAELKKPGSSVKISCKSSGYAFSS | 130 | DVVMTQSPLSLPVTLGQPASISCKSSQSLLESDGN | 307 |
| | QWMNWVKQRPGQGLEWIGQIYPEDGDTHYNG | | TYLNWLQQRPGQSPRRLIYLVDRLDSGVPDRFSGS | |
| | KFKGKATLTADKSTSTAYMELSSLTSEDSAV | | GSGTDFTLKISRVEAEDVGVYYCWQGTDLPQTFGG | |
| | YFCSSSNWVGSYWGQGTLVTVSS | | GTKVEIK | |
| D74P | QVQLVQSGAELKKPGSSVKISCKSSGYAFSS | 131 | DVVMTQSPLSLPVILGQPASISCKSSQSLLEADGN | 308 |
| | QWMNWVKQRPGQGLEWIGQIYPGDNDTHYND | | TYLNWLQQRPGQSPRRLIYLVDRLDSGVPDRFSGS | |
| | KFKGKATLTADKSTSTAYMELSSLTSEDSAV | | GSGTDFTLKISRVEAEDVGVYYCWQGTDLPQTFGG | |
| | YFCSSSNWVGSYWGQGTLVTVSS | | GTKVEIK | |
| E94P | QVQLVQSGAELKKPGSSVKISCKSSGYAFSS | 132 | DVVMTQSPLSLPVILGQPASISCKSSQSLLEEDGN | 309 |
| | QWMNWVKQRPGQGLEWIGQIYPGDGDTHMNG | | TYLNWLQQRPGQSPRRLIYLVDRLDSGVPDRFSGS | |
| | KFKGKATLTADKSTSTAYMELSSLTSEDSAV | | GSGTDFTLKISRVEAEDVGVYYCWQGTDLPQTFGG | |
| | YFCSSSNWVGSYWGQGTLVTVSS | | GTKVEIK | |
| F103P | QVQXVQSGAELKKPGSSVKISCKSSGYAFSS | 133 | DVVMTQSPLSLPVTLGQPASISCKSSQSLLDSDGN | 310 |
| | QWMNWVKQRPGQGLEWIGQIYPEDGDTHYNG | | TYLNWLQQRPGQSPRRLIYLVDRLDSGVPDRFSGS | |
| | KFKGKATLTADKSTSTAYMELSSLTSEDSAV | | GSGTDFTLKISRVEAEDVGVYYCWQGTDLPQTFGG | |
| | YFCSSSNWVGSYWGQGTLVTVSS | | GTKVEIK | |
| F64P | QVQLVQSGAELKKPGSSVKISCKSSGYAFSS | 134 | DVVMTQSPLSLPVTLGQPASISCKSSQSLLESDGN | 311 |
| | YYMNWVKQRPGQGLEWIGQIYPGDGDTHMNG | | TYLNWLQQRPGQSPRRLIYLVDRLDSGVPDRFSGS | |
| | KFKGKATLTADKSTSTAYMELSSLTSEDSAV | | GSGTDFTLKISRVEAEDVGVYYCWQGTDLPQTFGG | |
| | YFCSSSNWVGSYWGQGTLVTVSS | | GTKVEIK | |
| G14P | QVQLVQSGAELKKPGSSVKISCKSSGYAFSS | 135 | DVVMTQSPLSLPVILGQPASISCKSSQSLLEEDGN | 312 |
| | QWMNWVKQRPGQGLEWIGQIYPGDGDTHYND | | TYLNWLQQRPGQSPRRLIYLVDRLDDGVPDRFSGS | |
| | KFKGKATLTADKSTSTAYMELSSLTSEDSAV | | GSGTDFTLKISRVEAEDVGVYYCWQGTDLPQTFGG | |
| | YFCSSSNWVGSYWGQGTLVTVSS | | GTKVEIK | |
| G24P | QVQLVQSGAELKKPGSSVKISCKSSGYAFSS | 136 | DVVMTQSPLSLPVTLGQPASISCKSSQSLLESDGN | 313 |
| | NWMNWVKQRPGQGLEWIGQIYPEDGDTHYND | | TYLNWLQQRPGQSPRRLIYLVDRLDSGVPDRFSGS | |
| | KFKGKATLTADKSTSTAYMELSSLTSEDSAV | | GSGTDFTLKISRVEAEDVGVYYCWQGTDLPQTFGG | |
| | YFCSSSNWVGSYWGQGTLVTVSS | | GTKVEIK | |
| A44P | QVQLVQSGAELKKPGSSVKISCKSSGYAFSS | 137 | DVVMTQSPLSLPVTLGQPASISCKSSQSLLESDGN | 314 |
| | QWMNWVKQRPGQGLEWIGQIYPEDGDTHYND | | TYLNWLQQRPGQSPRRLIYLVNRLDSGVPDRFSGS | |
| | KFKGKATLTADKSTSTAYMELSSLTSEDSAV | | GSGTDFTLKISRVEAEDVGVYYCWQGTDLPQTFGG | |
| | YFCSSSNWVGSYWGQGTLVTVSS | | GTKVEIK | |
| A14P | QVQLVQSGAELKKPGSSVKISCKSSGYAFSS | 138 | DVVMTQSPLSLPVILGQPASISCKSSQSLLESDGN | 315 |
| | QWMNWVKQRPGQGLEWIGQIYPEDGDTHYNG | | TYLNWLQQRPGQSPRRLIYLVDRLDSGVPDRFSGS | |
| | KFEGKATLTADKSTSTAYMELSSLTSEDSAV | | GSGTDFTLKISRVEAEDVGVYYCWQGTDLPQTFGG | |
| | YFCSSSNWVGSYWGQGTLVTVSS | | GTKVEIK | |
| A94P | QVQLVQSGAELKKPGSSVKISCKSSGYAFSS | 139 | DVVMTQSPLSLPVTLGQPASISCKSSQSLLEEDGN | 316 |
| | QWMNWVKQRPGQGLEWIGQIYPDDGDTHYND | | TYLNWLQQRPGQSPRRLIYLVSRLDDGVPDRESGS | |
| | KFKGKATLTADKSTSTAYMELSSLTSEDSAV | | GSGTDFTLKISRVEAEDVGVYYCWQGTDLPQTFGG | |
| | YFCSSANWVGSYWGQGTLVTVSS | | GTKVEIK | |
| C24P | QVQLVQSGAELKKPGSSVKISCKSSGYAFSS | 140 | DVVMTQSPLSLPVTLGQPASISCKSSQSLLESDGN | 317 |
| | YYMNWVKQRPGQGLEWIGQIYPGDGDTHMNG | | TYLNWLQQRPGQSPRRLIYLVDRLDSGVPDRFSGS | |
| | KFDGKATLTADKSTSTAYMELSSLTSEDSAV | | GSGTDFTLKISRVEAEDVGVYYCWQGTDLPQTFGG | |
| | YFCSSSNWVGSYWGQGTLVTVSS | | GTKVEIK | |
| C54P | QVQLVQSGAELKKPGSSVKISCKSSGYAFSS | 141 | DVVMTQSPLSLPVILGQPASISCKSSQSLLEEDGN | 318 |
| | QWMNWVKQRPGQGLEWIGQIYPEDGDTHYND | | TYLNWLQQRPGQSPRRLIYLVSRLDIGVPDRESGS | |
| | KFKGKATLTADKSTSTAYMELSSLTSEDSAV | | GSGTDFTLKISRVEAEDVGVYYCWQGSHLPQTFGG | |
| | YFCSSSNWVGSYWGQGTLVTVSS | | GTKVEIK | |
| C84P | QVQLVQSGAELKKPGSSVKISCKSSGYAFSS | 142 | DVVMTQSPLSLPVTLGQPASISCKSSQSLLEEDGN | 319 |
| | QWMNWVKQRPGQGLEWIGQIYPEDGDTHYND | | TYLNWLQQRPGQSPRRLIYLVNRLDSGVPDRESGS | |
| | KFKGKATLTADKSTSTAYMELSSLTSEDSAV | | GSGTDFTLKISRVEAEDVGVYYCWQGTDLPQTFQG | |
| | YFCSSSNWVGSYWGQGTLVTVSS | | GTKVEIK | |
| D24P | QVQLVQSGAELKKPGSSVKISCKSSGYAFSS | 143 | DVVMTQSPLSLPVILGQPASISCKSSQSLLESDGN | 320 |
| | QWMNWVKQRPGQGLEWIGQIYPEDGDTHYNG | | TYLNWLQQRPGQSPRRLIYLVSRLDIGVPDRFSGS | |
| | KFKGKATLTADKSTSTAYMELSSLTSEDSAV | | GSGTDFTLKISRVEAEDVGVYYCWQGTDLPQTFGG | |
| | YFCSSSNWVGSYWGQGTLVTVSS | | GTKVEIK | |
| D34P | QVQLVQSGAELKKPGSSVKISCKSSGYAFSS | 144 | DVVMTQSPLSLPVTLGQPASISCKSSQSLLESDGN | 321 |
| | QWMNWVKQRPGQGLEWIGQIYPLDGDTHYND | | TYLNWLQQRPGQSPRRLIYLVDRLDSGVPDRFSGS | |
| | KFKGKATLTADKSTSTAYMELSSLTSEDSAV | | GSGTDFTLKISRVEAEDVGVYYCWQGTDLPQTFGG | |
| | YFCSSSNWVGSYWGQGTLVTVSS | | GTKVEIK | |
| D54P | QVQLVQSGAELKKPGSSVKISCKSSGYAFSS | 145 | DVVMTQSPLSLPVTLGQPASISCKSSQSLLEEDGN | 322 |
| | QWMNWVKQRPGQGLEWIGQIYPGDGDIHYNG | | TYLNWLQQRPGQSPRRLIYLVDRLDSGVPDRFSGS | |
| | KFKGKATLTADKSTSTAYMELSSLTSEDSAV | | GSGTDFTLKISRVEAEDVGVYYCWQGTDLPQTFGG | |
| | YFCSSSNWVGSYWGQGTLVTVSS | | GTKVEIK | |
| D94P | QVQLVQSGAELKKPGSSVKISCKSSGYAFSS | 146 | DVVMTQSPLSLPVILGQPASISCKASQSLLESDGN | 323 |
| | QWMNWVKQRPGQGLEWIGQIYPEDGDTHYNG | | TYLNWLQQRPGQSPRRLIYLVDRLDSGVPDRFSGS | |
| | KFKGKATLTADKSTSTAYMELSSLTSEDSAV | | GSGTDFTLKISRVEAEDVGVYYCWQGTDLPQTFGG | |
| | YFCSSSNWVGSYWGQGTLVTVSS | | GTKVEIK | |
| E14P | QVQLVQSGAELKKPGSSVKISCKSSGYAFSS | 147 | DVVMTQSPLSLPVTLGQPASISCKSSQSLLESDGN | 324 |
| | QWMNWVKQRPGQGLEWIGQIYPGDGDTHMNE | | TYLNWLQQRPGQSPRRLIYLVDRLDIGVPDRFSGS | |
| | KFKGKATLTADKSTSTAYMELSSLTSEDSAV | | GSGTDFTLKISRVEAEDVGVYYCWQGTDLPQTFGG | |
| | YFCSSSNWVGSYWGQGTLVTVSS | | GTKVEIK | |
| E54P | QVQLVQSGAELKKPGSSVKISCKSSGYAFSS | 148 | DVVMTQSPLSLPVTLGQPASISCKSSQSLLESDGN | 325 |
| | QWMNWVKQRPGQGLEWIGQIYPEDGDTHYNG | | TYLNWLQQRPGQSPRRLIYLVNRLDSGVPDRESGS | |
| | KFKGKATLTADKSTSTAYMELSSLTSEDSAV | | GSGTDFTLKISRVEAEDVGVYYCWQGTDLPQTFGG | |
| | YFCSSSNWVGSYWGQGTLVTVSS | | GTKVEIK | |
| G104P | QVQLVQSGAELKKPGSSVKISCKSSGYAFSS | 149 | DVVMTQSPLSLPVTLGQPASISCKSSQSLLEEDGN | 326 |
| | QWMNWVKQRPGQGLEWIGQIYPGDGDTHMNG | | TYLNWLQQRPGQSPRRLIYLVDRLDDGVPDRFSGS | |
| | KFKGKATLTADKSTSTAYMELSSLTSEDSAV | | GSGTDFTLKISRVEAEDVGVYYCWQGTDLPQTFGG | |
| | YFCSSSNWVGSYWGQGTLVTVSS | | GTKVEIK | |
| G124P | QVQLVQSGAELKKPGSSVKISCKSSGYAFSS | 150 | DVVMTQSPLSLPVILGQPASISCKSSQSLLDSDGN | 327 |
| | QWMNWVKQRPGQGLEWIGQIYPEDGDTHYND | | TYLNWLQQRPGQSPRRLIYLVSRLDIGVPDRFSGS | |
| | KFKGKATLTADKSTSTAYMELSSLTSEDSAV | | GSGTDFTLKISRVEAEDVGVYYCWQGTDLPQTFGG | |
| | YFCSSSNWVGSYWGQGTLVTVSS | | GTKVEIK | |
| H14P | QVQLVQSGAELKKPGSSVKISCKSSGYAFSS | 151 | DVVMTQSPLSLPVILGQPASISCHSSQSLLEEDGN | 328 |
| | QWMNWVKQRPGQGLEWIGQIYPGDGDTHMNG | | TYLNWLQQRPGQSPRRLIYLVDRLDSGVPDRFSGS | |
| | KFKGKATLTADKSTSTAYMELSSLTSEDSAV | | GSGTDFTLKISRVEAEDVGVYYCWQGTDLPQTFGG | |
| | YFCSSSNWVGSYWGQGTLVTVSS | | GTKVEIK | |
| H54P | QVQLVQSGAELKKPGSSVKISCKSSGYAFSS | 152 | DVVMTQSPLSLPVTLGQPASISCHSSQSLLESDGN | 329 |
| | QWMNWVKQRPGQGLEWIGQIYPEDGDTHYNG | | TYLNWLQQRPGQSPRRLIYLVNRLDDGVPDRFSGS | |
| | KFKGKATLTADKSTSTAYMELSSLTSEDSAV | | GSGTDFTLKISRVEAEDVGVYYCWQGTDLPQTFGG | |
| | YFCSSSNWVGSYWGQGTLVTVSS | | GTKVEIK | |
| AS3P | QVQLVQSGAELKKPGSSVKISCKSSGYAFSS | 153 | DVVMTQSPLSLPVILGQPASISCKSSQSLLDSDGN | 330 |
| | QWMNWVKQRPGQGLEWIGQIYPEDGDTHYNG | | TYLNWLQQRPGQSPRRLIYLVDRLDSGVPDRFSGS | |
| | KFRGKATLTADKSTSTAYMELSSLTSEDSAV | | GSGTDFTLKISRVEAEDVGVYYCWQGTDLPQTFGG | |
| | YFCSSSNWVGSYWGQGTLVTVSS | | GTKVEIK | |
| B113P | QXXXVQSGAELKKPGSSVKISCKSSGYAFSS | 154 | DVVMTQSPLSLPVILGQPASISCHSSQSLLESDGN | 331 |
| | NWMNWVKQRPGQGLEWIGQIYPGDGDVHYNG | | TYLNWLQQRPGQSPRRLIYLVSRRDSGVPDRFSGS | |
| | KFKGKATLTADKSTSTAYMELSSLTSEDSAV | | GSGTDFTLKISRVEAEDVGVYYCWQGTDLPQTFGG | |
| | YFCSSANWVGSYWGQGTLVTVSS | | GTKVEIK | |
| C33P | QVQLVQSGAELKKPGSSVKISCKSSGYAFSS | 155 | DVVMTQSPLSLPVILGQPASISCKSSQSLLESDGN | 332 |
| | QWMNWVKQRPGQGLEWIGQIYPLDGDTHYNG | | TYLNWLQQRPGQSPRRLIYLVDRLDSGVPDRESGS | |
| | KFNGKATLTADKSTSTAYMELSSLTSEDSAV | | GSGTDFTLKISRVEAEDVGVYYCWQGTDLPQTFGG | |
| | YFCSSSNWVGSYWGQGTLVTVSS | | GTKVEIK | |
| D113P | QVQLVQSGAELKKPGSSVKISCKSSGYAFSS | 156 | DVVMTQSPLSLPVTLGQPASISCKSSQSLLEGDGN | 333 |
| | QWMNWVKQRPGQGLEWIGQIYPGDGDTHMNG | | TYLNWLQQRPGQSPRRLIYLVDRLDIGVPDRESGS | |
| | KFKGKATLTADKSTSTAYMELSSLTSEDSAV | | GSGTDFTLKISRVEAEDVGVYYCWQGTDLPQTFGG | |
| | YFCSSSNWVGSYWGQGTLVTVSS | | GTKVEIK | |
| D53P | QVQLVQSGAELKKPGSSVKISCKSSGYAFSS | 157 | DVVMTQSPLSLPVILGQPASISCKSSQSLLESDGN | 334 |
| | QWMNWVKQRPGQGLEWIGQIYPGDGDTHMNG | | TYLNWLQQRPGQSPRRLIYLVDRLDSGVPDRFSGS | |
| | KFKGKATLTADKSTSTAYMELSSLTSEDSAV | | GSGTDFTLKISRVEAEDVGVYYCWQGTDLPQTFGG | |
| | YFCSSSNWVGSYWGQGTLVTVSS | | GTKVEIK | |
| E93P | QVQXVQSGAELKKPGSSVKISCKSSGYAFSS | 158 | DVVMTQSPLSLPVILGQPASISCKSSQSLLDSDGN | 335 |
| | QWMNWVKQRPGQGLEWIGQIYPGDGDTHMNG | | TYLNWLQQRPGQSPRRLIYLVDRLDSGVPDRFSGS | |
| | KFKGKATLTADKSTSTAYMELSSLTSEDSAV | | GSGTDFTLKISRVEAEDVGVYYCWQGTDLPQIFGG | |
| | YFCSSSNWVGSYWGQGTLVTVSS | | GTKVEIK | |
| F23P | QVQLVQSGAELKKPGSSVKISCKSSGYAFSS | 159 | DVVMTQSPLSLPVTLGQPASISCKSSQSLLESDGN | 336 |
| | QWMNWVKQRPGQGLEWIGQIYPLDGDTHMNG | | TYLNWLQQRPGQSPRRLIYLVDRLDSGVPDRFSGS | |
| | KFKGKATLTADKSTSTAYMELSSLTSEDSAV | | GSGTDFTLKISRVEAEDVGVYYCWQGSHLPQTFGG | |
| | YFCSSSNWVGSYWGQGTLVTVSS | | GTKVEIK | |
| G113P | QVQLVQSGAELKKPGSSVKISCKSSGYAFSS | 160 | DVVMTQSPLSLPVILGQPASISCKSSQSLLESDGL | 337 |
| | QWMNWVKQRPGQGLEWIGQIYPGDGDTHYND | | TYLNWLQQRPGQSPRRLIYLVDRLDSGVPDRESGS | |
| | KFKGKATLTADKSTSTAYMELSSLTSEDSAV | | GSGTDFTLKISRVEAEDVGVYYCWQGTDLPQTFGG | |
| | YFCSSSNWVGSYWGQGTLVTVSS | | GTKVEIK | |
| H93P | QVQLVQSGAELKKPGSSVKISCKSSGYAFSS | 161 | DVVMTQSPLSLPVILGQPASISCKSSQSLLESDGN | 338 |
| | QWMNWVKQRPGQGLEWIGQIYPGDGDTHYND | | TYLNWLQQRPGQSPRRLIYLVDRLDSGVPDRFSGS | |
| | KFKGKATLTADKSTSTAYMELSSLTSEDSAV | | GSGTDFTLKISRVEAEDVGVYYCWQGTDLPQTFGG | |
| | YFCSSSNWVGSYWGQGTLVTVSS | | GTKVEIK | |
| G104P | QVQLVQSGAELKKPGSSVKISCKSSGYAFSS | 149 | DVVMTQSPLSLPVILGQPASISCKSSQSLLEEDGN | 326 |
| | QWMNWVKQRPGQGLEWIGQIYPGDGDTHMNG | | TYLNWLQQRPGQSPRRLIYLVDRLDDGVPDRESGS | |
| | KFKGKATLTADKSTSTAYMELSSLTSEDSAV | | GSGTDFTLKISRVEAEDVGVYYCWQGTDLPQTFGG | |
| | YFCSSSNWVGSYWGQGTLVTVSS | | GTKVEIK | |
| D113P | QVQLVQSGAELKKPGSSVKISCKSSGYAFSS | 156 | DVVMTQSPLSLPVILGQPASISCKSSQSLLEGDGN | 333 |
| | QWMNWVKQRPGQGLEWIGQIYPGDGDTHMNG | | TYLNWLQQRPGQSPRRLIYLVDRLDIGVPDRESGS | |
| | KFKGKATLTADKSTSTAYMELSSLTSEDSAV | | GSGTDFTLKISRVEAEDVGVYYCWQGTDLPQTFGG | |
| | YFCSSSNWVGSYWGQGTLVTVSS | | GTKVEIK | |
| E93P | QVQXVQSGAELKKPGSSVKISCKSSGYAFSS | 158 | DVVMTQSPLSLPVILGQPASISCKSSQSLLDSDGN | 335 |
| | QWMNWVKQRPGQGLEWIGQIYPGDGDTHMNG | | TYLNWLQQRPGQSPRRLIYLVDRLDSGVPDRFSGS | |
| | KFKGKATLTADKSTSTAYMELSSLTSEDSAV | | GSGTDFTLKISRVEAEDVGVYYCWQGTDLPQTFGG | |
| | YFCSSSNWVGSYWGQGTLVTVSS | | GTKVEIK | |
| D24P | QVQLVQSGAELKKPGSSVKISCKSSGYAFSS | 143 | DVVMTQSPLSLPVTLGQPASISCKSSQSLLESDGN | 320 |
| | QWMNWVKQRPGQGLEWIGQIYPEDGDTHYNG | | TYLNWLQQRPGQSPRRLIYLVSRLDIGVPDRFSGS | |
| | KFKGKATLTADKSTSTAYMELSSLTSEDSAV | | GSGTDFTLKISRVEAEDVGVYYCWQGTDLPQTFGG | |
| | YFCSSSNWVGSYWGQGTLVTVSS | | GTKVEIK | |

TABLE 3
Exemplary anti-SCF scFv sequences

| | SEQ ID |
|---|---|
| Clone | NO |
| A84P | 481 |
| A64P | 482 |
| B114P | 483 |
| A104P | 484 |
| B124P | 485 |
| H74P | 486 |
| H63P | 487 |
| H83P | 488 |
| F54P | 489 |
| D44P | 490 |
| D14P | 491 |
| B104P | 492 |
| B23P | 493 |
| B34P | 494 |
| B53P | 495 |
| C104P | 496 |
| D124P | 497 |
| D74P | 498 |
| E94P | 499 |
| F103P | 500 |
| F64P | 501 |
| G14P | 502 |
| G24P | 503 |
| A44P | 515 |
| A14P | 522 |
| A94P | 524 |
| C24P | 509 |
| C54P | 481 |
| C84P | 482 |
| D24P | 483 |
| D34P | 484 |
| D54P | 485 |
| D94P | 486 |
| E14P | 487 |
| E54P | 488 |
| G104P | 489 |
| G124P | 490 |
| H14P | 491 |
| H54P | 492 |
| A53P | 493 |
| B113P | 494 |
| C33P | 495 |
| D113P | 496 |
| D53P | 497 |
| E93P | 498 |
| F23P | 499 |
| G113P | 500 |
| H93P | 501 |
| G104P | 502 |
| D113P | 503 |
| E93P | 515 |
| D24P | 522 |

TABLE 4
Exemplary anti-SCF antibodies having an IgG4 domain
comprising a constant heavy domain of SEQ ID NO: 1440
and a constant light domain of SEQ ID NO: 1442

| | Antibodies having an IgG4 | | Antibodies having an IgG4 | |
|---|---|---|---|---|
| | domain comprising a constant | | domain comprising a constant | |
| | heavy domain of SEQ ID NO: | | heavy domain of SEQ ID NO: | |
| | 1440 and a constant light | | 1441 and a constant light | |
| | domain of SEQ ID NO: 1442 | | domain of SEQ ID NO: 1442 | |
| | Heavy Chain | Light Chain | Heavy Chain | Light Chain |
| Clone | SEQ ID NO. | SEQ ID NO: | SEQ ID NO. | SEQ ID NO: |
| A84P | 618 | 755 | 892 | 1029 |
| A64P | 619 | 756 | 893 | 1030 |
| B114P | 620 | 757 | 894 | 1031 |
| A104P | 621 | 758 | 895 | 1032 |
| B124P | 622 | 759 | 896 | 1033 |
| H74P | 623 | 760 | 897 | 1034 |
| H63P | 624 | 761 | 898 | 1035 |
| H83P | 625 | 762 | 899 | 1036 |
| F54P | 626 | 763 | 900 | 1037 |
| D44P | 627 | 764 | 901 | 1038 |
| D14P | 628 | 765 | 902 | 1039 |
| B104P | 629 | 766 | 903 | 1040 |
| B23P | 630 | 767 | 904 | 1041 |
| B34P | 631 | 768 | 905 | 1042 |
| B53P | 632 | 769 | 906 | 1043 |
| C104P | 633 | 770 | 907 | 1044 |
| D124P | 634 | 771 | 908 | 1045 |
| D74P | 635 | 772 | 909 | 1046 |
| E94P | 636 | 773 | 910 | 1047 |
| F103P | 637 | 774 | 911 | 1048 |
| F64P | 638 | 775 | 912 | 1049 |
| G14P | 639 | 776 | 913 | 1050 |
| G24P | 640 | 777 | 914 | 1051 |
| A44P | 652 | 789 | 926 | 1063 |
| A14P | 659 | 796 | 933 | 1070 |
| A94P | 661 | 798 | 935 | 1072 |
| C24P | 646 | 783 | 920 | 1057 |
| C54P | 618 | 755 | 892 | 1029 |
| C84P | 619 | 756 | 893 | 1030 |
| D24P | 620 | 757 | 894 | 1031 |
| D34P | 621 | 758 | 895 | 1032 |
| D54P | 622 | 759 | 896 | 1033 |
| D94P | 623 | 760 | 897 | 1034 |
| E14P | 624 | 761 | 898 | 1035 |
| E54P | 625 | 762 | 899 | 1036 |
| G104P | 626 | 763 | 900 | 1037 |
| G124P | 627 | 764 | 901 | 1038 |
| H14P | 628 | 765 | 902 | 1039 |
| H54P | 629 | 766 | 903 | 1040 |
| A53P | 630 | 767 | 904 | 1041 |
| B113P | 631 | 768 | 905 | 1042 |
| C33P | 632 | 769 | 906 | 1043 |
| D113P | 633 | 770 | 907 | 1044 |
| D53P | 634 | 771 | 908 | 1045 |
| E93P | 635 | 772 | 909 | 1046 |
| F23P | 636 | 773 | 910 | 1047 |
| G113P | 637 | 774 | 911 | 1048 |
| H93P | 638 | 775 | 912 | 1049 |
| G104P | 639 | 776 | 913 | 1050 |
| D113P | 640 | 777 | 914 | 1051 |
| E93P | 652 | 789 | 926 | 1063 |
| D24P | 659 | 796 | 933 | 1070 |

The present disclosure provides antibodies having modified CDR regions that surprisingly resulted in improved affinity for SCF relative to the parental murine antibody 5H10 as well as the parental humanized antibody 5H10 VK3/VH1. Modifications in the CDR regions of the parental antibodies were identified that surprisingly result in significantly enhanced affinity for SCF. Table 5 provides consensus sequences representing the modified CDRs.

TABLE 5
CDR consensus sequences

| | SEQ ID | | Amino acid at variable |
|---|---|---|---|
| | NO | Sequence | position(s) |
| HCDR1 | 1 | SX₂X₃MN | X₂ is Q, N, or Y |
| | | | X₃ is W or Y |
| | 7 | SX₂WMN | X₂ is Q or N |
| HCDR2 | 2 | QIYPX₅DX₇DX₉HX₁₁ | X₅ is E, G, D, or L; X₇ is G, |
| | | NX₁₃KFX₁₆X₁₇ | D or N; X₉ is T or I; Xu is M |
| | | | or Y; X₁₃ is G, D, or E; X₁₆ is |
| | | | K, R, N, E, or D; and X₁₇ is |
| | | | G or T |
| | 12 | QIYPX₅DX₇DX₉HX₁₁ | X₅ is E, G, D, or L; X₇ is G, |
| | | NX₁₃KFKX₁₇ | D or N; X₉ is T or I; Xu is M |
| | | | or Y; X₁₃ is Gor D; and X₁₇ |
| | | | is G or T |
| | 8 | QIYPX₅DX-DX₉HX₁₁ | X₅ is E, G, or D; X₇ is G or |
| | | NX₁₃KFKX₁₇ | D; X₉ is T or I; X₁₃ is G or D; |
| | | | X₁₁ is M or Y; and X₁₇ is G |
| | | | or T |
| HCDR3 | 3 | XINWX₄GSY | X₁ is S or A; and X₄ is V or D |
| | 9 | SNWX₄GSY | X₄ is V or D |
| LCDR1 | 4 | X₁X₂SQSLLX₈X₉DGN | X₁ is K or H; X₂ is S or A; X₈ |
| | | TYLN | is E or D; and X₉ is S, E, Q, |
| | | | A, or G |
| | 13 | KSSQSLLX₈X₉DGNTY | X₈ is E or D; X₉ is S, E, Q, |
| | | LN | or A |
| | 10 | KSSQSLLEX₉DGNTYL | X₉ is S, E, Q, or A |
| | | N | |
| LCDR2 | 5 | LVX₃RX₅DX₇ | X₃ is D, N, or S; X₅ is L or R; |
| | | | and X₇ is I, D, S, or L |
| | 11 | LVX₃RLDX₇ | X₃ is D or N; X₇ is I, D, S, or L |
| LCDR3 | 6 | WQGX₄X₅LPQT | X₄ is T or S; and X₅ is D or H |

In some embodiments, the antibody or fragment thereof comprises a heavy chain CDR1 (hCDR1) comprising an amino acid sequence selected from the group consisting of SEQ ID Nos. 22-24. In some embodiments, the antibody or fragment thereof comprises a heavy chain CDR1 (hCDR1) comprising an amino acid sequence selected from the group consisting of SEQ ID Nos. 22-25.

In some embodiments, the antibody or fragment thereof comprises a heavy chain CDR2 (hCDR2) comprising an amino acid sequence selected from the group consisting of SEQ ID Nos. 26-36. In some embodiments, the antibody or fragment thereof comprises a heavy chain CDR2 (hCDR2) comprising an amino acid sequence selected from the group consisting of SEQ ID Nos. 15, 26-66.

In some embodiments, the antibody or fragment thereof comprises a heavy chain CDR3 (hCDR3) comprising an amino acid sequence selected from the group consisting of SEQ ID Nos. 16 or 67. In some embodiments, the antibody or fragment thereof comprises a heavy chain CDR3 (hCDR3) comprising an amino acid sequence selected from the group consisting of SEQ ID Nos. 16 and 67-70.

In some embodiments, the antibody or fragment thereof comprises a light chain CDR1 (lCDR1) comprising an amino acid sequence selected from the group consisting of SEQ ID Nos. 71-75 and 79. In some embodiments, the antibody or fragment thereof comprises a light chain CDR1 (lCDR1) comprising an amino acid sequence selected from the group consisting of SEQ ID Nos. 71-89.

In some embodiments, the antibody or fragment thereof comprises comprise a light chain CDR2 (lCDR2) comprising an amino acid sequence selected from the group consisting of SEQ ID Nos. 90-96 and 98. In some embodiments, the antibody or fragment thereof comprises a light chain CDR2 (lCDR2) comprising an amino acid sequence selected from the group consisting of SEQ ID Nos. 18 and 90-110.

In some embodiments, the antibody or fragment thereof comprises comprise a light chain CDR3 (lCDR3) comprising an amino acid sequence selected from the group consisting of SEQ ID Nos. 111 and 112. In some embodiments, the antibody or fragment thereof comprises comprise a light chain CDR3 (lCDR3) comprising an amino acid sequence selected from the group consisting of SEQ ID Nos. 111-113.

In some embodiments, the antibody or fragment thereof comprises a heavy chain variable region comprising an amino acid sequence selected from the group consisting of SEQ ID Nos. 114-137, 143, 149, 156, and 158. In some embodiments, the antibody or fragment thereof comprises a heavy chain variable region comprising an amino acid sequence selected from the group consisting of SEQ ID Nos. 114-290. In some embodiments, the antibody or fragment thereof comprises a heavy chain variable region having at least 80% identity to an amino acid sequence selected from the group consisting of SEQ ID Nos. 114-137, 143, 149, 156, and 158. In some embodiments, the antibody or fragment thereof comprises a heavy chain variable region having at least 80% identity to an amino acid sequence selected from the group consisting of SEQ ID Nos. 114-290. In some embodiments, the antibody or fragment thereof comprises a heavy chain variable region having at least 85% identity to an amino acid sequence selected from the group consisting of SEQ ID Nos. 114-137, 143, 149, 156, and 158. In some embodiments, the antibody or fragment thereof comprises a heavy chain variable region having at least 85% identity to an amino acid sequence selected from the group consisting of SEQ ID Nos. 114-290. In some embodiments, the antibody or fragment thereof comprises a heavy chain variable region having at least 90% identity to an amino acid sequence selected from the group consisting of SEQ ID Nos. 114-137, 143, 149, 156, and 158. In some embodiments, the antibody or fragment thereof comprises a heavy chain variable region having at least 90% identity to an amino acid sequence selected from the group consisting of SEQ ID Nos. 114-290. In some embodiments, the antibody or fragment thereof comprises a heavy chain variable region having at least 95% identity to an amino acid sequence selected from the group consisting of SEQ ID Nos. 114-137, 143, 149, 156, and 158. In some embodiments, the antibody or fragment thereof comprises a heavy chain variable region having at least 95% identity to an amino acid sequence selected from the group consisting of SEQ ID Nos. 114-290. In some embodiments, the antibody or fragment thereof comprises a heavy chain variable region having at least 97% identity to an amino acid sequence selected from the group consisting of SEQ ID Nos. 114-137, 143, 149, 156, and 158. In some embodiments, the antibody or fragment thereof comprises a heavy chain variable region having at least 97% identity to an amino acid sequence selected from the group consisting of SEQ ID Nos. 114-290. In some embodiments, the antibody or fragment thereof comprises a heavy chain variable region having at least 99% identity to an amino acid sequence selected from the group consisting of SEQ ID Nos. 114-137, 143, 149, 156, and 158. In some embodiments, the antibody or fragment thereof comprises a heavy chain variable region having at least 99% identity to an amino acid sequence selected from the group consisting of SEQ ID Nos. 114-290.

In some embodiments, the antibody or fragment thereof comprises a light chain variable region comprising an amino acid sequence selected from the group consisting of SEQ ID Nos. 291-314, 320, 326, 333, and 335. In some embodiments, the antibody or fragment thereof comprises a light chain variable region comprising an amino acid sequence selected from the group consisting of SEQ ID Nos. 291-467. In some embodiments, the antibody or fragment thereof comprises a light chain variable region having at least 80% identity to an amino acid sequence selected from the group consisting of SEQ ID Nos. 291-314, 320, 326, 333, and 335. In some embodiments, the antibody or fragment thereof comprises a light chain variable region having at least 80% identity to an amino acid sequence selected from the group consisting of SEQ ID Nos. SEQ ID Nos. 291-467. In some embodiments, the antibody or fragment thereof comprises a light chain variable region having at least 85% identity to an amino acid sequence selected from the group consisting of SEQ ID Nos. 291-314, 320, 326, 333, and 335. In some embodiments, the antibody or fragment thereof comprises a light chain variable region having at least 85% identity to an amino acid sequence selected from the group consisting of SEQ ID Nos. SEQ ID Nos. 291-467. In some embodiments, the antibody or fragment thereof comprises a light chain variable region having at least 90% identity to an amino acid sequence selected from the group consisting of SEQ ID Nos. 291-314, 320, 326, 333, and 335. In some embodiments, the antibody or fragment thereof comprises a light chain variable region having at least 90% identity to an amino acid sequence selected from the group consisting of SEQ ID Nos. SEQ ID Nos. 291-467. In some embodiments, the antibody or fragment thereof comprises a light chain variable region having at least 95% identity to an amino acid sequence selected from the group consisting of SEQ ID Nos. 291-314, 320, 326, 333, and 335. In some embodiments, the antibody or fragment thereof comprises a light chain variable region having at least 95% identity to an amino acid sequence selected from the group consisting of SEQ ID Nos. SEQ ID Nos. 291-467. In some embodiments, the antibody or fragment thereof comprises a light chain variable region having at least 97% identity to an amino acid sequence selected from the group consisting of SEQ ID Nos. 291-314, 320, 326, 333, and 335. In some embodiments, the antibody or fragment thereof comprises a light chain variable region having at least 97% identity to an amino acid sequence selected from the group consisting of SEQ ID Nos. SEQ ID Nos. 291-467. In some embodiments, the antibody or fragment thereof comprises a light chain variable region having at least 99% identity to an amino acid sequence selected from the group consisting of SEQ ID Nos. 291-314, 320, 326, 333, and 335. In some embodiments, the antibody or fragment thereof comprises a light chain variable region having at least 99% identity to an amino acid sequence selected from the group consisting of SEQ ID Nos. SEQ ID Nos. 291-467.

In some embodiments, the antibody or fragment thereof is any one of those provided in Table 3 or Table 4, or an antibody or fragment thereof having at least 80%, at least 85%, at least 90%, at least 95%, at least 97%, or at least 99% sequence identity to any one of those provided in Table or Table 4. In some embodiments, the antibody or fragment thereof comprises a heavy chain comprising an amino acid sequence selected from the group consisting of SEQ ID Nos. 618-754 and a light chain comprising an amino acid sequence selected from the group consisting of SEQ ID NO: 755-891. In some embodiments, the antibody or fragment thereof comprises a heavy chain comprising an amino acid sequence selected from the group consisting of SEQ ID Nos. 618-655 and a light chain comprising an amino acid sequence selected from the group consisting of SEQ ID NO: 755-801. In some embodiments, the antibody or fragment thereof comprises a heavy chain comprising an amino acid sequence selected from the group consisting of SEQ ID Nos. 618-640, 652, 659, 661, and 646 and a light chain comprising an amino acid sequence selected from the group consisting of SEQ ID NO: 755-777, 789, 796, 798, and 783. In some embodiments, the antibody or fragment thereof comprises a heavy chain comprising an amino acid sequence having at least 80%, at least 85%, at least 90%, at least 95%, at least 97%, or at least 99% sequence identity to any one of SEQ ID Nos. 618-754 and a light chain comprising an amino acid sequence having at least 80%, at least 85%, at least 90%, at least 95%, at least 97%, or at least 99% sequence identity to any one of SEQ ID NO: 755-891.

In some embodiments, the antibody or fragment thereof comprises a heavy chain comprising an amino acid sequence selected from the group consisting of SEQ ID Nos. 892-1028 and a light chain comprising an amino acid sequence selected from the group consisting of SEQ ID NO: 1029-1165. In some embodiments, the antibody or fragment thereof comprises a heavy chain comprising an amino acid sequence selected from the group consisting of SEQ ID Nos. 892-938 and a light chain comprising an amino acid sequence selected from the group consisting of SEQ ID NO: 1029-1075. In some embodiments, the antibody or fragment thereof comprises a heavy chain comprising an amino acid sequence selected from the group consisting of SEQ ID NOs. 892-914 and 926, 933, 935, and 920 and a light chain comprising an amino acid sequence selected from the group consisting of SEQ ID NO: 1029-1051 and 1063, 1070, 1072, and 1057. In some embodiments, the antibody or fragment thereof comprises a heavy chain comprising an amino acid sequence having at least 80%, at least 85%, at least 90%, at least 95%, at least 97%, or at least 99% sequence identity to any one of SEQ ID Nos. 892-1028 and a light chain comprising an amino acid sequence having at least 80%, at least 85%, at least 90%, at least 95%, at least 97%, or at least 99% sequence identity to any one of SEQ ID NO: 1029-1165.

In some embodiments, the antibody or fragment thereof comprises a heavy chain comprising an amino acid sequence encoded by any one of SEQ ID NOs. 1166-1302. In some embodiments, the antibody or fragment thereof comprises a light chain comprising an amino acid sequence encoded by any one of SEQ ID NOs. 1303-1439.

In some embodiments, the antibody or fragment thereof provided herein comprise herein provided CDRs, and a variable region provided herein or a variant thereof. Variants may comprise 1, 2, 3, 4, 5, 6, 7, 8, 9, or 10 amino acid substitutions or deletions, or a combination thereof. In some embodiments, the amino acid substitutions are conservative substitutions.

In some embodiments, the present disclosure provides antibodies or fragments thereof that have high affinity for SCF, for example, affinity in the range of about 1 nM to about 20 nM, about 1 nM to about 10 nM, about 1 nM to about 5 nM, or about 1 nM to about 4 nM, about 2 nM to about 5 nM, about 2 nM to about 4 nM, about 2 nM to about 20 nM, or about 2 nM to about 10 nM. In some embodiments, the affinity of the antibodies or fragments thereof for SCF is less than about 5 nM, less than about 6 nM, less than about 7 nM, less than about 8 nM, less than about 9 nM, less than about 10 nM, less than about 15 nM, or less than about 20 nM. The affinity of exemplary antibodies or fragments thereof is provided in Tables A2 and A3 of Example 2.

In some embodiments, the present disclosure provides antibodies or fragments thereof that specifically bind to a region of the amino acid sequence provided herein as SEQ ID NO: 470 with an affinity (K_{D}) of about 20 nM or lower (e.g., about 20 nM, about 19 nM, about 18 nM, about 17 nM, about 16 nM, about 15 nM, about 14 nM, about 13 nM, about 12 nM, about 11 nM, about 10 nM, about 9 nM, about 8 nM, about 7 nM, about 6 nM, about 5 nM, about 4 nM, about 3 nM, about 2 nM, or about 1 nM), or about 15 nM or lower (e.g., about 15 nM, about 14 nM, about 13 nM, about 12 nM, about 11 nM, about 10 nM, about 9 nM, about 8 nM, about 7 nM, about 6 nM, about 5 nM, about 4 nM, about 3 nM, about 2 nM, or about 1 nM), or about 10 nM or lower (e.g., about 10 nM, about 9 nM, about 8 nM, about 7 nM, about 6 nM, about 5 nM, about 4 nM, about 3 nM, about 2 nM, or about 1 nM), or about 8 nM or lower (e.g., about 8 nM, about 7 nM, about 6 nM, about 5 nM, about 4 nM, about 3 nM, about 2 nM, or about 1 nM), or about 6 nM or lower (e.g., about 6 nM, about 5 nM, about 4 nM, about 3 nM, about 2 nM, or about 1 nM), about 5 nM or lower (e.g., about 5 nM, about 4 nM, about 3 nM, about 2 nM, or about 1 nM), or about 4 nM or lower (e.g., about 4 nM, about 3 nM, about 2 nM, or about 1 nM). In some embodiments, the antibodies or fragments thereof provided herein specifically bind to an epitope comprising the amino acid sequence of SEQ ID NO: 471 (ASSLRNDSSSSNRK) or SEQ ID NO: 472 ASSLRNDSSSSNR) with an affinity (K_{D}) of about 20 nM or lower (e.g., about 20 nM, about 19 nM, about 18 nM, about 17 nM, about 16 nM, about 15 nM, about 14 nM, about 13 nM, about 12 nM, about 11 nM, about 10 nM, about 9 nM, about 8 nM, about 7 nM, about 6 nM, about 5 nM, about 4 nM, about 3 nM, about 2 nM, or about 1 nM), or about 15 nM or lower (e.g., about 15 nM, about 14 nM, about 13 nM, about 12 nM, about 11 nM, about 10 nM, about 9 nM, about 8 nM, about 7 nM, about 6 nM, about 5 nM, about 4 nM, about 3 nM, about 2 nM, or about 1 nM), or about 10 nM or lower (e.g., about 10 nM, about 9 nM, about 8 nM, about 7 nM, about 6 nM, about 5 nM, about 4 nM, about 3 nM, about 2 nM, or about 1 nM), or about 8 nM or lower (e.g., about 8 nM, about 7 nM, about 6 nM, about 5 nM, about 4 nM, about 3 nM, about 2 nM, or about 1 nM), or about 6 nM or lower (e.g., about 6 nM, about 5 nM, about 4 nM, about 3 nM, about 2 nM, or about 1 nM), about 5 nM or lower (e.g., about 5 nM, about 4 nM, about 3 nM, about 2 nM, or about 1 nM), or about 4 nM or lower (e.g., about 4 nM, about 3 nM, about 2 nM, or about 1 nM). In some embodiments, the present disclosure provides antibodies or fragments thereof that specifically bind to an epitope comprising at least 5, at least 6, at least 7, at least 8, at least 9, or at least 10 contiguous amino acids of SEQ ID NO: 471, with an affinity (K_{D}) of about 20 nM or lower (e.g., about 20 nM, about 19 nM, about 18 nM, about 17 nM, about 16 nM, about 15 nM, about 14 nM, about 13 nM, about 12 nM, about 11 nM, about 10 nM, about 9 nM, about 8 nM, about 7 nM, about 6 nM, about 5 nM, about 4 nM, about 3 nM, about 2 nM, or about 1 nM), or about 15 nM or lower (e.g., about 15 nM, about 14 nM, about 13 nM, about 12 nM, about 11 nM, about 10 nM, about 9 nM, about 8 nM, about 7 nM, about 6 nM, about 5 nM, about 4 nM, about 3 nM, about 2 nM, or about 1 nM), or about 10 nM or lower (e.g., about 10 nM, about 9 nM, about 8 nM, about 7 nM, about 6 nM, about 5 nM, about 4 nM, about 3 nM, about 2 nM, or about 1 nM), or about 8 nM or lower (e.g., about 8 nM, about 7 nM, about 6 nM, about 5 nM, about 4 nM, about 3 nM, about 2 nM, or about 1 nM), or about 6 nM or lower (e.g., about 6 nM, about 5 nM, about 4 nM, about 3 nM, about 2 nM, or about 1 nM), about 5 nM or lower (e.g., about 5 nM, about 4 nM, about 3 nM, about 2 nM, or about 1 nM), or about 4 nM or lower (e.g., about 4 nM, about 3 nM, about 2 nM, or about 1 nM).

In some embodiments, the antibodies described herein bind to a polypeptide comprising the amino acid sequence of SEQ ID NO: 479. The 5H10 VK3/VH1 humanized antibody of Table 1 binds to a C-terminal biotinylated polypeptide of SEQ ID NO: 479 with a K_{D} of about 165.6. The 5H10 VK3/VH1 humanized antibody of Table 1 binds to an N-terminal biotinylated polypeptide of SEQ ID NO: 479 with a K_{D} of about 229.6.

Binding of the antibodies may be evaluated via ELISA, for example, as described in Example 3 of this disclosure. The absorbance values for binding of the humanized 5H10 VK3/VH1 antibody of Table 1 to the polypeptide of SEQ ID NO: 479 (a peptide of SCF248) at various antibody concentrations are below.

| | 1000 | 100 | 5 | 1 | 0.5 | 0.1 |
|---|---|---|---|---|---|---|
| | ng/mL | ng/mL | ng/mL | ng/mL | ng/mL | ng/mL |
| 5H10 | 3.29 | 2.12 | 0.33 | 0.24 | 0.19 | 0.17 |
| VK3/VH1 | | | | | | |
| humanized | | | | | | |

In some embodiments, the absorbance value for binding of an antibody described herein to a polypeptide of SEQ ID NO: 479 is greater than 2.12 at an antibody concentration of 100 ng/mL, for example, at least about 2.2, at least about 2.3, at least about 2.4, at least about 2.5, at least about 2.6, at least about 2.7, at least about 2.8, at least about 2.9, at least about 3.0, at least about 3.1, at least about 3.2, at least about 3.3, at least about 3.4, at least about 3.5, at least about 3.6, or at least about 3.7. In some embodiments, the absorbance value for binding of an antibody described herein to a polypeptide of SEQ ID NO: 479 is greater than about 0.33 at an antibody concentration of 5 ng/mL, for example, at least about 0.4, at least about 0.5, at least about 0.6, at least about 0.7, at least about 0.8, at least about 0.9, at least about 1, at least about 1.1, at least about 1.2, at least about 1.3, at least about 1.4, at least about 1.5, at least about 1.6, at least about 1.7, at least about 1.8, at least about 1.9, or at least about 2.0. In some embodiments, the absorbance value for binding of an antibody described herein to a polypeptide of SEQ ID NO: 479 is greater than about 0.24 at an antibody concentration of 1 ng/mL, for example, at least about 0.3, at least about 0.4, at least about 0.5, at least about 0.6, at least about 0.7, at least about 0.8, at least about 9, or at least about 1. In some embodiments, the absorbance value for binding of an antibody described herein to a polypeptide of SEQ ID NO: 479 is greater than about 0.19 at an antibody concentration of 0.5 ng/mL, for example, at least about 0.2, at least about 0.3, or at least about 0.4. In some embodiments, the absorbance value for binding of an antibody described herein to a polypeptide of SEQ ID NO: 479 is greater than about 0.17 at an antibody concentration of 0.1 ng/mL, for example, at least about 0.2, at least about 0.3, or at least about 0.4.

In embodiments, provided herein are antibodies that bind to a polypeptide of SEQ ID NO: 479 more effectively than the 5H10 VK3/VH1 humanized antibody of Table 1. In embodiments, the absorbance value for binding of the antibody is at least about 50% higher, at least about 100% higher, at least about 150% higher, at least about 200% higher, at least about 250% higher, at least about 300% higher, at least about 350% higher, at least about 400% higher, at least about 450% higher, at least about 500% higher than the absorbance value for binding of the 5H10 VK3/VH1 humanized antibody at the same concentration. In embodiments, the absorbance values for binding of the antibody are measured at 1000 nanograms (ng) of antibody per milliliter (mL) of solution, 100 ng/mL, 5 ng/mL, 1 ng/mL, 0.5 ng/mL, 0.1 ng/mL, or any concentration therebetween.

In some embodiments, the antibodies and fragments thereof comprise amino acids 31-35, 50-66, and 99-105 of any one of the heavy chain variable regions provided herein, as defined by the Kabat numbering scheme. In some embodiments, the antibodies and fragments thereof comprise amino acids 24-39, 55-61, and 94-102 of any one of the light chain variable regions provided herein, as defined by the Kabat numbering scheme.

Exemplary humanized antibodies are provided herein. Additional anti-SCF antibodies comprising the heavy and light chain CDRs provided herein may be generated using any human framework sequence, and are also encompassed in the present invention. In one embodiment, framework sequences suitable for use in the present invention include those framework sequences that are structurally similar to the framework sequences provided herein. Further modifications in the framework regions may be made to improve the properties of the antibodies provided herein. Such further framework modifications may include chemical modifications; point mutations to reduce immunogenicity or remove T cell epitopes; or back mutation to the residue in the original germline sequence.

In some embodiments, such framework modifications include those corresponding to the mutations exemplified herein, including backmutations to the germline sequence. For example, in one embodiment, one or more amino acids in the human framework regions of the VH and/or VL of the humanized antibodies provided herein are back mutated to the corresponding amino acid in the parent murine antibody. The present invention also encompasses humanized antibodies that bind to SCF (e.g., SCF248) and comprise framework modifications corresponding to the exemplary modifications described herein with respect to any suitable framework sequence, as well as other framework modifications that otherwise improve the properties of the antibodies. In other embodiments, the antibodies provided herein comprise one or more mutations to improve stability, improve solubility, alter glycosylation, and/or reduce immunogenicity, such as, for example, by targeted amino acid changes that reduce deamidation or oxidation, reduce isomerization, optimize the hydrophobic core and/or charge cluster residues, remove hydrophobic surface residues, optimize residues involved in the interface between the variable heavy and variable light chains, and/or modify the isoelectric point.

The anti-SCF antibodies and fragments thereof provided herein may further comprise Fc region modifications to alter effector functions. Fc modifications may be amino acid insertions, deletions, or substitutions, or may be chemical modifications. For example, Fc region modifications may be made to increase or decrease complement binding, to increase or decrease antibody-dependent cellular cytoxicity, or to increase or decrease the half-life of the antibody. Some Fc modifications increase or decrease the affinity of the antibody for an Fcγ receptor such as FcγRI, FcγRII, FcγRIII, or FcRn. Various Fc modifications have been described in the art, for example, in Shields et al., J Biol. Chem 276; 6591 (2001); Tai et al. Blood 119; 2074 (2012); Spiekermann et al. J Exp. Med 196; 303 (2002); Moore et al. mAbs 2:2; 181 (2010); Medzihradsky Methods in Molecular Biology 446; 293 (2008); Mannan et al. Drug Metabolism and Disposition 35; 86 (2007); and Idusogie et al. J Immunol 164; 4178 (2000). In some embodiments, Fc region glycosylation patters are altered. In other embodiments, the Fc region is modified by pegylation (e.g., by reacting the antibody or fragment thereof with polyethylene glycol (PEG). Exemplary Fc modifications include modifications at one or more amino acid position selected from the group consisting of 228, 233, 234, 235, 236, 241, 248, 265, 297, 309, 331, and 409 (Kabat numbering; Kabat et al., Sequences of Immunological Interest, Fifth Edition, National Institute of Health, Bethesda, Md. (1991)). In embodiments, the antibody has modifications to reduce or abolish effector function. In embodiments, the antibody is an IgG1 antibody having one or more Fc modification selected from the group consisting of E233P, L234V, L234A, L235V, L235A, G236(deleted), D265A, D270A, N297A and N297Q. In embodiments, the antibody is an IgG4 antibody having one or more Fc modification selected from the group consisting of S228P, E233P, F234A, F234V, L235A, L235V, S241P, L248E, D265A, D265T, L309L, and R409K. In embodiments, the anti-SCF antibodies are IgG4 antibodies having a S241P mutation and an L248E mutation.

In embodiments, the present disclosure provides antibodies provided herein that comprise a human IgG4 heavy chain constant region according to SEQ ID NO: 1441 and a human IgG4 light chain region according to SEQ ID NO: 1442. In embodiments, the present disclosure provides antibodies provided herein that comprise a human IgG4 heavy chain constant region encoded by a nucleic acid sequence according to SEQ ID NO: 1441 and a human IgG4 light chain region encoding a nucleic acid sequence according to SEQ ID NO: 1442. In embodiments, the present disclosure provides antibodies provided herein that comprise a human IgG4 heavy chain constant region according to SEQ ID NO: 1440 and a human IgG4 light chain region according to SEQ ID NO: 1442. In embodiments, the present disclosure provides antibodies provided herein that comprise a human IgG4 heavy chain constant region encoded by a nucleic acid sequence according to SEQ ID NO: 1440 and a human IgG4 light chain region encoding a nucleic acid sequence according to SEQ ID NO: 1442. In embodiments, the present disclosure provides antibodies comprising a heavy chain that is at least about 80%, at least about 85%, at least about 90%, at least about 95%, or about 99% sequence identity to SEQ ID NOs: 892-938 and a light chain that is at least about 80%, at least about 85%, at least about 90%, at least about 95%, or about 99% sequence identity to any one of SEQ ID Nos: 1029-1075. In embodiments, the present disclosure provides antibodies comprising a heavy chain that is at least about 80%, at least about 85%, at least about 90%, at least about 95%, or about 99% sequence identity to SEQ ID NOs: 892-1028 and a light chain that is at least about 80%, at least about 85%, at least about 90%, at least about 95%, or about 99% sequence identity to any one of SEQ ID Nos: 1029-1165. In embodiments, the present disclosure provides antibodies comprising a heavy chain that is at least about 80%, at least about 85%, at least about 90%, at least about 95%, or about 99% sequence identity to SEQ ID NOs: 618-664 and a light chain that is at least about 80%, at least about 85%, at least about 90%, at least about 95%, or about 99% sequence identity to any one of SEQ ID Nos: 755-801. In embodiments, the present disclosure provides antibodies comprising a heavy chain that is at least about 80%, at least about 85%, at least about 90%, at least about 95%, or about 99% sequence identity to SEQ ID NOs: 618-754 and a light chain that is at least about 80%, at least about 85%, at least about 90%, at least about 95%, or about 99% sequence identity to any one of SEQ ID Nos: 755-891.

In embodiments, the disclosure provides an antibody or antigen binding fragment thereof comprising: a heavy chain variable region comprising an amino acid sequence corresponding to SEQ ID NO: 114 and a light chain variable region comprising an amino acid sequence corresponding to SEQ ID NO: 291. In embodiments, the disclosure provides an antibody or antigen binding fragment thereof comprising: a heavy chain variable region comprising an amino acid sequence corresponding to SEQ ID NO: 115 and a light chain variable region comprising an amino acid sequence corresponding to SEQ ID NO: 292. In embodiments, the disclosure provides an antibody or antigen binding fragment thereof comprising: a heavy chain variable region comprising an amino acid sequence corresponding to SEQ ID NO: 116 and a light chain variable region comprising an amino acid sequence corresponding to SEQ ID NO: 293. In embodiments, the disclosure provides an antibody or antigen binding fragment thereof comprising: a heavy chain variable region comprising an amino acid sequence corresponding to SEQ ID NO: 117 and a light chain variable region comprising an amino acid sequence corresponding to SEQ ID NO: 294. In embodiments, the disclosure provides an antibody or antigen binding fragment thereof comprising: a heavy chain variable region comprising an amino acid sequence corresponding to SEQ ID NO: 118 and a light chain variable region comprising an amino acid sequence corresponding to SEQ ID NO: 295. In embodiments, the disclosure provides an antibody or antigen binding fragment thereof comprising: a heavy chain variable region comprising an amino acid sequence corresponding to SEQ ID NO: 119 and a light chain variable region comprising an amino acid sequence corresponding to SEQ ID NO: 296. In embodiments, the disclosure provides an antibody or antigen binding fragment thereof comprising: a heavy chain variable region comprising an amino acid sequence corresponding to SEQ ID NO: 120 and a light chain variable region comprising an amino acid sequence corresponding to SEQ ID NO: 297. In embodiments, the disclosure provides an antibody or antigen binding fragment thereof comprising: a heavy chain variable region comprising an amino acid sequence corresponding to SEQ ID NO: 121 and a light chain variable region comprising an amino acid sequence corresponding to SEQ ID NO: 298. In embodiments, the disclosure provides an antibody or antigen binding fragment thereof comprising: a heavy chain variable region comprising an amino acid sequence corresponding to SEQ ID NO: 122 and a light chain variable region comprising an amino acid sequence corresponding to SEQ ID NO: 299. In embodiments, the disclosure provides an antibody or antigen binding fragment thereof comprising: a heavy chain variable region comprising an amino acid sequence corresponding to SEQ ID NO: 123 and a light chain variable region comprising an amino acid sequence corresponding to SEQ ID NO: 300. In embodiments, the disclosure provides an antibody or antigen binding fragment thereof comprising: a heavy chain variable region comprising an amino acid sequence corresponding to SEQ ID NO: 124 and a light chain variable region comprising an amino acid sequence corresponding to SEQ ID NO: 301. In embodiments, the disclosure provides an antibody or antigen binding fragment thereof comprising: a heavy chain variable region comprising an amino acid sequence corresponding to SEQ ID NO: 125 and a light chain variable region comprising an amino acid sequence corresponding to SEQ ID NO: 302. In embodiments, the disclosure provides an antibody or antigen binding fragment thereof comprising: a heavy chain variable region comprising an amino acid sequence corresponding to SEQ ID NO: 126 and a light chain variable region comprising an amino acid sequence corresponding to SEQ ID NO: 303. In embodiments, the disclosure provides an antibody or antigen binding fragment thereof comprising: a heavy chain variable region comprising an amino acid sequence corresponding to SEQ ID NO: 127 and a light chain variable region comprising an amino acid sequence corresponding to SEQ ID NO: 304. In embodiments, the disclosure provides an antibody or antigen binding fragment thereof comprising: a heavy chain variable region comprising an amino acid sequence corresponding to SEQ ID NO: 128 and a light chain variable region comprising an amino acid sequence corresponding to SEQ ID NO: 305. In embodiments, the disclosure provides an antibody or antigen binding fragment thereof comprising: a heavy chain variable region comprising an amino acid sequence corresponding to SEQ ID NO: 129 and a light chain variable region comprising an amino acid sequence corresponding to SEQ ID NO: 306. In embodiments, the disclosure provides an antibody or antigen binding fragment thereof comprising: a heavy chain variable region comprising an amino acid sequence corresponding to SEQ ID NO: 130 and a light chain variable region comprising an amino acid sequence corresponding to SEQ ID NO: 307. In embodiments, the disclosure provides an antibody or antigen binding fragment thereof comprising: a heavy chain variable region comprising an amino acid sequence corresponding to SEQ ID NO: 131 and a light chain variable region comprising an amino acid sequence corresponding to SEQ ID NO: 308. In embodiments, the disclosure provides an antibody or antigen binding fragment thereof comprising: a heavy chain variable region comprising an amino acid sequence corresponding to SEQ ID NO: 132 and a light chain variable region comprising an amino acid sequence corresponding to SEQ ID NO: 309. In embodiments, the disclosure provides an antibody or antigen binding fragment thereof comprising: a heavy chain variable region comprising an amino acid sequence corresponding to SEQ ID NO: 133 and a light chain variable region comprising an amino acid sequence corresponding to SEQ ID NO: 310. In embodiments, the disclosure provides an antibody or antigen binding fragment thereof comprising: a heavy chain variable region comprising an amino acid sequence corresponding to SEQ ID NO: 134 and a light chain variable region comprising an amino acid sequence corresponding to SEQ ID NO: 311. In embodiments, the disclosure provides an antibody or antigen binding fragment thereof comprising: a heavy chain variable region comprising an amino acid sequence corresponding to SEQ ID NO: 135 and a light chain variable region comprising an amino acid sequence corresponding to SEQ ID NO: 312. In embodiments, the disclosure provides an antibody or antigen binding fragment thereof comprising: a heavy chain variable region comprising an amino acid sequence corresponding to SEQ ID NO: 136 and a light chain variable region comprising an amino acid sequence corresponding to SEQ ID NO: 313. In embodiments, the disclosure provides an antibody or antigen binding fragment thereof comprising: a heavy chain variable region comprising an amino acid sequence corresponding to SEQ ID NO: 137 and a light chain variable region comprising an amino acid sequence corresponding to SEQ ID NO: 314. In embodiments, the disclosure provides an antibody or antigen binding fragment thereof comprising: a heavy chain variable region comprising an amino acid sequence corresponding to SEQ ID NO: 149 and a light chain variable region comprising an amino acid sequence corresponding to SEQ ID NO: 326. In embodiments, the disclosure provides an antibody or antigen binding fragment thereof comprising: a heavy chain variable region comprising an amino acid sequence corresponding to SEQ ID NO: 156 and a light chain variable region comprising an amino acid sequence corresponding to SEQ ID NO: 333. In embodiments, the disclosure provides an antibody or antigen binding fragment thereof comprising: a heavy chain variable region comprising an amino acid sequence corresponding to SEQ ID NO: 158 and a light chain variable region comprising an amino acid sequence corresponding to SEQ ID NO: 335. In embodiments, the disclosure provides an antibody or antigen binding fragment thereof comprising: a heavy chain variable region comprising an amino acid sequence corresponding to SEQ ID NO: 143 and a light chain variable region comprising an amino acid sequence corresponding to SEQ ID NO: 320.

In embodiments, the disclosure provides an antibody or antigen binding fragment thereof comprising: a heavy chain variable region comprising an amino acid sequence having at least 80% identity to SEQ ID NO: 114 and a light chain variable region comprising an amino acid sequence having at least 80% identity to SEQ ID NO: 291. In embodiments, the disclosure provides an antibody or antigen binding fragment thereof comprising: a heavy chain variable region comprising an amino acid sequence having at least 80% identity to SEQ ID NO: 115 and a light chain variable region comprising an amino acid sequence having at least 80% identity to SEQ ID NO: 292. In embodiments, the disclosure provides an antibody or antigen binding fragment thereof comprising: a heavy chain variable region comprising an amino acid sequence having at least 80% identity to SEQ ID NO: 116 and a light chain variable region comprising an amino acid sequence having at least 80% identity to SEQ ID NO: 293. In embodiments, the disclosure provides an antibody or antigen binding fragment thereof comprising: a heavy chain variable region comprising an amino acid sequence having at least 80% identity to SEQ ID NO: 117 and a light chain variable region comprising an amino acid sequence having at least 80% identity to SEQ ID NO: 294. In embodiments, the disclosure provides an antibody or antigen binding fragment thereof comprising: a heavy chain variable region comprising an amino acid sequence having at least 80% identity to SEQ ID NO: 118 and a light chain variable region comprising an amino acid sequence having at least 80% identity to SEQ ID NO: 295. In embodiments, the disclosure provides an antibody or antigen binding fragment thereof comprising: a heavy chain variable region comprising an amino acid sequence having at least 80% identity to SEQ ID NO: 119 and a light chain variable region comprising an amino acid sequence having at least 80% identity to SEQ ID NO: 296. In embodiments, the disclosure provides an antibody or antigen binding fragment thereof comprising: a heavy chain variable region comprising an amino acid sequence having at least 80% identity to SEQ ID NO: 120 and a light chain variable region comprising an amino acid sequence having at least 80% identity to SEQ ID NO: 297. In embodiments, the disclosure provides an antibody or antigen binding fragment thereof comprising: a heavy chain variable region comprising an amino acid sequence having at least 80% identity to SEQ ID NO: 121 and a light chain variable region comprising an amino acid sequence having at least 80% identity to SEQ ID NO: 298. In embodiments, the disclosure provides an antibody or antigen binding fragment thereof comprising: a heavy chain variable region comprising an amino acid sequence having at least 80% identity to SEQ ID NO: 122 and a light chain variable region comprising an amino acid sequence having at least 80% identity to SEQ ID NO: 299. In embodiments, the disclosure provides an antibody or antigen binding fragment thereof comprising: a heavy chain variable region comprising an amino acid sequence having at least 80% identity to SEQ ID NO: 123 and a light chain variable region comprising an amino acid sequence having at least 80% identity to SEQ ID NO: 300. In embodiments, the disclosure provides an antibody or antigen binding fragment thereof comprising: a heavy chain variable region comprising an amino acid sequence having at least 80% identity to SEQ ID NO: 124 and a light chain variable region comprising an amino acid sequence having at least 80% identity to SEQ ID NO: 301. In embodiments, the disclosure provides an antibody or antigen binding fragment thereof comprising: a heavy chain variable region comprising an amino acid sequence having at least 80% identity to SEQ ID NO: 125 and a light chain variable region comprising an amino acid sequence having at least 80% identity to SEQ ID NO: 302. In embodiments, the disclosure provides an antibody or antigen binding fragment thereof comprising: a heavy chain variable region comprising an amino acid sequence having at least 80% identity to SEQ ID NO: 126 and a light chain variable region comprising an amino acid sequence having at least 80% identity to SEQ ID NO: 303. In embodiments, the disclosure provides an antibody or antigen binding fragment thereof comprising: a heavy chain variable region comprising an amino acid sequence having at least 80% identity to SEQ ID NO: 127 and a light chain variable region comprising an amino acid sequence having at least 80% identity to SEQ ID NO: 304. In embodiments, the disclosure provides an antibody or antigen binding fragment thereof comprising: a heavy chain variable region comprising an amino acid sequence having at least 80% identity to SEQ ID NO: 128 and a light chain variable region comprising an amino acid sequence having at least 80% identity to SEQ ID NO: 305. In embodiments, the disclosure provides an antibody or antigen binding fragment thereof comprising: a heavy chain variable region comprising an amino acid sequence having at least 80% identity to SEQ ID NO: 129 and a light chain variable region comprising an amino acid sequence having at least 80% identity to SEQ ID NO: 306. In embodiments, the disclosure provides an antibody or antigen binding fragment thereof comprising: a heavy chain variable region comprising an amino acid sequence having at least 80% identity to SEQ ID NO: 130 and a light chain variable region comprising an amino acid sequence having at least 80% identity to SEQ ID NO: 307. In embodiments, the disclosure provides an antibody or antigen binding fragment thereof comprising: a heavy chain variable region comprising an amino acid sequence having at least 80% identity to SEQ ID NO: 131 and a light chain variable region comprising an amino acid sequence having at least 80% identity to SEQ ID NO: 308. In embodiments, the disclosure provides an antibody or antigen binding fragment thereof comprising: a heavy chain variable region comprising an amino acid sequence having at least 80% identity to SEQ ID NO: 132 and a light chain variable region comprising an amino acid sequence having at least 80% identity to SEQ ID NO: 309. In embodiments, the disclosure provides an antibody or antigen binding fragment thereof comprising: a heavy chain variable region comprising an amino acid sequence having at least 80% identity to SEQ ID NO: 133 and a light chain variable region comprising an amino acid sequence having at least 80% identity to SEQ ID NO: 310. In embodiments, the disclosure provides an antibody or antigen binding fragment thereof comprising: a heavy chain variable region comprising an amino acid sequence having at least 80% identity to SEQ ID NO: 134 and a light chain variable region comprising an amino acid sequence having at least 80% identity to SEQ ID NO: 311. In embodiments, the disclosure provides an antibody or antigen binding fragment thereof comprising: a heavy chain variable region comprising an amino acid sequence having at least 80% identity to SEQ ID NO: 135 and a light chain variable region comprising an amino acid sequence having at least 80% identity to SEQ ID NO: 312. In embodiments, the disclosure provides an antibody or antigen binding fragment thereof comprising: a heavy chain variable region comprising an amino acid sequence having at least 80% identity to SEQ ID NO: 136 and a light chain variable region comprising an amino acid sequence having at least 80% identity to SEQ ID NO: 313. In embodiments, the disclosure provides an antibody or antigen binding fragment thereof comprising: a heavy chain variable region comprising an amino acid sequence having at least 80% identity to SEQ ID NO: 137 and a light chain variable region comprising an amino acid sequence having at least 80% identity to SEQ ID NO: 314. In embodiments, the disclosure provides an antibody or antigen binding fragment thereof comprising: a heavy chain variable region comprising an amino acid sequence having at least 80% identity to SEQ ID NO: 149 and a light chain variable region comprising an amino acid sequence having at least 80% identity to SEQ ID NO: 326. In embodiments, the disclosure provides an antibody or antigen binding fragment thereof comprising: a heavy chain variable region comprising an amino acid sequence having at least 80% identity to SEQ ID NO: 156 and a light chain variable region comprising an amino acid sequence having at least 80% identity to SEQ ID NO: 333. In embodiments, the disclosure provides an antibody or antigen binding fragment thereof comprising: a heavy chain variable region comprising an amino acid sequence having at least 80% identity to SEQ ID NO: 158 and a light chain variable region comprising an amino acid sequence having at least 80% identity to SEQ ID NO: 335. In embodiments, the disclosure provides an antibody or antigen binding fragment thereof comprising: a heavy chain variable region comprising an amino acid sequence having at least 80% identity to SEQ ID NO: 143 and a light chain variable region comprising an amino acid sequence having at least 80% identity to SEQ ID NO: 320.

In embodiments, the disclosure provides an antibody or antigen binding fragment thereof comprising: a heavy chain variable region comprising an amino acid sequence having at least 90% identity to SEQ ID NO: 114 and a light chain variable region comprising an amino acid sequence having at least 90% identity to SEQ ID NO: 291. In embodiments, the disclosure provides an antibody or antigen binding fragment thereof comprising: a heavy chain variable region comprising an amino acid sequence having at least 90% identity to SEQ ID NO: 115 and a light chain variable region comprising an amino acid sequence having at least 90% identity to SEQ ID NO: 292. In embodiments, the disclosure provides an antibody or antigen binding fragment thereof comprising: a heavy chain variable region comprising an amino acid sequence having at least 90% identity to SEQ ID NO: 116 and a light chain variable region comprising an amino acid sequence having at least 90% identity to SEQ ID NO: 293. In embodiments, the disclosure provides an antibody or antigen binding fragment thereof comprising: a heavy chain variable region comprising an amino acid sequence having at least 90% identity to SEQ ID NO: 117 and a light chain variable region comprising an amino acid sequence having at least 90% identity to SEQ ID NO: 294. In embodiments, the disclosure provides an antibody or antigen binding fragment thereof comprising: a heavy chain variable region comprising an amino acid sequence having at least 90% identity to SEQ ID NO: 118 and a light chain variable region comprising an amino acid sequence having at least 90% identity to SEQ ID NO: 295. In embodiments, the disclosure provides an antibody or antigen binding fragment thereof comprising: a heavy chain variable region comprising an amino acid sequence having at least 90% identity to SEQ ID NO: 119 and a light chain variable region comprising an amino acid sequence having at least 90% identity to SEQ ID NO: 296. In embodiments, the disclosure provides an antibody or antigen binding fragment thereof comprising: a heavy chain variable region comprising an amino acid sequence having at least 90% identity to SEQ ID NO: 120 and a light chain variable region comprising an amino acid sequence having at least 90% identity to SEQ ID NO: 297. In embodiments, the disclosure provides an antibody or antigen binding fragment thereof comprising: a heavy chain variable region comprising an amino acid sequence having at least 90% identity to SEQ ID NO: 121 and a light chain variable region comprising an amino acid sequence having at least 90% identity to SEQ ID NO: 298. In embodiments, the disclosure provides an antibody or antigen binding fragment thereof comprising: a heavy chain variable region comprising an amino acid sequence having at least 90% identity to SEQ ID NO: 122 and a light chain variable region comprising an amino acid sequence having at least 90% identity to SEQ ID NO: 299. In embodiments, the disclosure provides an antibody or antigen binding fragment thereof comprising: a heavy chain variable region comprising an amino acid sequence having at least 90% identity to SEQ ID NO: 123 and a light chain variable region comprising an amino acid sequence having at least 90% identity to SEQ ID NO: 300. In embodiments, the disclosure provides an antibody or antigen binding fragment thereof comprising: a heavy chain variable region comprising an amino acid sequence having at least 90% identity to SEQ ID NO: 124 and a light chain variable region comprising an amino acid sequence having at least 90% identity to SEQ ID NO: 301. In embodiments, the disclosure provides an antibody or antigen binding fragment thereof comprising: a heavy chain variable region comprising an amino acid sequence having at least 90% identity to SEQ ID NO: 125 and a light chain variable region comprising an amino acid sequence having at least 90% identity to SEQ ID NO: 302. In embodiments, the disclosure provides an antibody or antigen binding fragment thereof comprising: a heavy chain variable region comprising an amino acid sequence having at least 90% identity to SEQ ID NO: 126 and a light chain variable region comprising an amino acid sequence having at least 90% identity to SEQ ID NO: 303. In embodiments, the disclosure provides an antibody or antigen binding fragment thereof comprising: a heavy chain variable region comprising an amino acid sequence having at least 90% identity to SEQ ID NO: 127 and a light chain variable region comprising an amino acid sequence having at least 90% identity to SEQ ID NO: 304. In embodiments, the disclosure provides an antibody or antigen binding fragment thereof comprising: a heavy chain variable region comprising an amino acid sequence having at least 90% identity to SEQ ID NO: 128 and a light chain variable region comprising an amino acid sequence having at least 90% identity to SEQ ID NO: 305. In embodiments, the disclosure provides an antibody or antigen binding fragment thereof comprising: a heavy chain variable region comprising an amino acid sequence having at least 90% identity to SEQ ID NO: 129 and a light chain variable region comprising an amino acid sequence having at least 90% identity to SEQ ID NO: 306. In embodiments, the disclosure provides an antibody or antigen binding fragment thereof comprising: a heavy chain variable region comprising an amino acid sequence having at least 90% identity to SEQ ID NO: 130 and a light chain variable region comprising an amino acid sequence having at least 90% identity to SEQ ID NO: 307. In embodiments, the disclosure provides an antibody or antigen binding fragment thereof comprising: a heavy chain variable region comprising an amino acid sequence having at least 90% identity to SEQ ID NO: 131 and a light chain variable region comprising an amino acid sequence having at least 90% identity to SEQ ID NO: 308. In embodiments, the disclosure provides an antibody or antigen binding fragment thereof comprising: a heavy chain variable region comprising an amino acid sequence having at least 90% identity to SEQ ID NO: 132 and a light chain variable region comprising an amino acid sequence having at least 90% identity to SEQ ID NO: 309. In embodiments, the disclosure provides an antibody or antigen binding fragment thereof comprising: a heavy chain variable region comprising an amino acid sequence having at least 90% identity to SEQ ID NO: 133 and a light chain variable region comprising an amino acid sequence having at least 90% identity to SEQ ID NO: 310. In embodiments, the disclosure provides an antibody or antigen binding fragment thereof comprising: a heavy chain variable region comprising an amino acid sequence having at least 90% identity to SEQ ID NO: 134 and a light chain variable region comprising an amino acid sequence having at least 90% identity to SEQ ID NO: 311. In embodiments, the disclosure provides an antibody or antigen binding fragment thereof comprising: a heavy chain variable region comprising an amino acid sequence having at least 90% identity to SEQ ID NO: 135 and a light chain variable region comprising an amino acid sequence having at least 90% identity to SEQ ID NO: 312. In embodiments, the disclosure provides an antibody or antigen binding fragment thereof comprising: a heavy chain variable region comprising an amino acid sequence having at least 90% identity to SEQ ID NO: 136 and a light chain variable region comprising an amino acid sequence having at least 90% identity to SEQ ID NO: 313. In embodiments, the disclosure provides an antibody or antigen binding fragment thereof comprising: a heavy chain variable region comprising an amino acid sequence having at least 90% identity to SEQ ID NO: 137 and a light chain variable region comprising an amino acid sequence having at least 90% identity to SEQ ID NO: 314. In embodiments, the disclosure provides an antibody or antigen binding fragment thereof comprising: a heavy chain variable region comprising an amino acid sequence having at least 90% identity to SEQ ID NO: 149 and a light chain variable region comprising an amino acid sequence having at least 90% identity to SEQ ID NO: 326. In embodiments, the disclosure provides an antibody or antigen binding fragment thereof comprising: a heavy chain variable region comprising an amino acid sequence having at least 90% identity to SEQ ID NO: 156 and a light chain variable region comprising an amino acid sequence having at least 90% identity to SEQ ID NO: 333. In embodiments, the disclosure provides an antibody or antigen binding fragment thereof comprising: a heavy chain variable region comprising an amino acid sequence having at least 90% identity to SEQ ID NO: 158 and a light chain variable region comprising an amino acid sequence having at least 90% identity to SEQ ID NO: 335. In embodiments, the disclosure provides an antibody or antigen binding fragment thereof comprising: a heavy chain variable region comprising an amino acid sequence having at least 90% identity to SEQ ID NO: 143 and a light chain variable region comprising an amino acid sequence having at least 90% identity to SEQ ID NO: 320.

In embodiments, the disclosure provides an antibody or antigen binding fragment thereof comprising: a heavy chain variable region comprising an amino acid sequence having at least 95% identity to SEQ ID NO: 114 and a light chain variable region comprising an amino acid sequence having at least 95% identity to SEQ ID NO: 291. In embodiments, the disclosure provides an antibody or antigen binding fragment thereof comprising: a heavy chain variable region comprising an amino acid sequence having at least 95% identity to SEQ ID NO: 115 and a light chain variable region comprising an amino acid sequence having at least 95% identity to SEQ ID NO: 292. In embodiments, the disclosure provides an antibody or antigen binding fragment thereof comprising: a heavy chain variable region comprising an amino acid sequence having at least 95% identity to SEQ ID NO: 116 and a light chain variable region comprising an amino acid sequence having at least 95% identity to SEQ ID NO: 293. In embodiments, the disclosure provides an antibody or antigen binding fragment thereof comprising: a heavy chain variable region comprising an amino acid sequence having at least 95% identity to SEQ ID NO: 117 and a light chain variable region comprising an amino acid sequence having at least 95% identity to SEQ ID NO: 294. In embodiments, the disclosure provides an antibody or antigen binding fragment thereof comprising: a heavy chain variable region comprising an amino acid sequence having at least 95% identity to SEQ ID NO: 118 and a light chain variable region comprising an amino acid sequence having at least 95% identity to SEQ ID NO: 295. In embodiments, the disclosure provides an antibody or antigen binding fragment thereof comprising: a heavy chain variable region comprising an amino acid sequence having at least 95% identity to SEQ ID NO: 119 and a light chain variable region comprising an amino acid sequence having at least 95% identity to SEQ ID NO: 296. In embodiments, the disclosure provides an antibody or antigen binding fragment thereof comprising: a heavy chain variable region comprising an amino acid sequence having at least 95% identity to SEQ ID NO: 120 and a light chain variable region comprising an amino acid sequence having at least 95% identity to SEQ ID NO: 297. In embodiments, the disclosure provides an antibody or antigen binding fragment thereof comprising: a heavy chain variable region comprising an amino acid sequence having at least 95% identity to SEQ ID NO: 121 and a light chain variable region comprising an amino acid sequence having at least 95% identity to SEQ ID NO: 298. In embodiments, the disclosure provides an antibody or antigen binding fragment thereof comprising: a heavy chain variable region comprising an amino acid sequence having at least 95% identity to SEQ ID NO: 122 and a light chain variable region comprising an amino acid sequence having at least 95% identity to SEQ ID NO: 299. In embodiments, the disclosure provides an antibody or antigen binding fragment thereof comprising: a heavy chain variable region comprising an amino acid sequence having at least 95% identity to SEQ ID NO: 123 and a light chain variable region comprising an amino acid sequence having at least 95% identity to SEQ ID NO: 300. In embodiments, the disclosure provides an antibody or antigen binding fragment thereof comprising: a heavy chain variable region comprising an amino acid sequence having at least 95% identity to SEQ ID NO: 124 and a light chain variable region comprising an amino acid sequence having at least 95% identity to SEQ ID NO: 301. In embodiments, the disclosure provides an antibody or antigen binding fragment thereof comprising: a heavy chain variable region comprising an amino acid sequence having at least 95% identity to SEQ ID NO: 125 and a light chain variable region comprising an amino acid sequence having at least 95% identity to SEQ ID NO: 302. In embodiments, the disclosure provides an antibody or antigen binding fragment thereof comprising: a heavy chain variable region comprising an amino acid sequence having at least 95% identity to SEQ ID NO: 126 and a light chain variable region comprising an amino acid sequence having at least 95% identity to SEQ ID NO: 303. In embodiments, the disclosure provides an antibody or antigen binding fragment thereof comprising: a heavy chain variable region comprising an amino acid sequence having at least 95% identity to SEQ ID NO: 127 and a light chain variable region comprising an amino acid sequence having at least 95% identity to SEQ ID NO: 304. In embodiments, the disclosure provides an antibody or antigen binding fragment thereof comprising: a heavy chain variable region comprising an amino acid sequence having at least 95% identity to SEQ ID NO: 128 and a light chain variable region comprising an amino acid sequence having at least 95% identity to SEQ ID NO: 305. In embodiments, the disclosure provides an antibody or antigen binding fragment thereof comprising: a heavy chain variable region comprising an amino acid sequence having at least 95% identity to SEQ ID NO: 129 and a light chain variable region comprising an amino acid sequence having at least 95% identity to SEQ ID NO: 306. In embodiments, the disclosure provides an antibody or antigen binding fragment thereof comprising: a heavy chain variable region comprising an amino acid sequence having at least 95% identity to SEQ ID NO: 130 and a light chain variable region comprising an amino acid sequence having at least 95% identity to SEQ ID NO: 307. In embodiments, the disclosure provides an antibody or antigen binding fragment thereof comprising: a heavy chain variable region comprising an amino acid sequence having at least 95% identity to SEQ ID NO: 131 and a light chain variable region comprising an amino acid sequence having at least 95% identity to SEQ ID NO: 308. In embodiments, the disclosure provides an antibody or antigen binding fragment thereof comprising: a heavy chain variable region comprising an amino acid sequence having at least 95% identity to SEQ ID NO: 132 and a light chain variable region comprising an amino acid sequence having at least 95% identity to SEQ ID NO: 309. In embodiments, the disclosure provides an antibody or antigen binding fragment thereof comprising: a heavy chain variable region comprising an amino acid sequence having at least 95% identity to SEQ ID NO: 133 and a light chain variable region comprising an amino acid sequence having at least 95% identity to SEQ ID NO: 310. In embodiments, the disclosure provides an antibody or antigen binding fragment thereof comprising: a heavy chain variable region comprising an amino acid sequence having at least 95% identity to SEQ ID NO: 134 and a light chain variable region comprising an amino acid sequence having at least 95% identity to SEQ ID NO: 311. In embodiments, the disclosure provides an antibody or antigen binding fragment thereof comprising: a heavy chain variable region comprising an amino acid sequence having at least 95% identity to SEQ ID NO: 135 and a light chain variable region comprising an amino acid sequence having at least 95% identity to SEQ ID NO: 312. In embodiments, the disclosure provides an antibody or antigen binding fragment thereof comprising: a heavy chain variable region comprising an amino acid sequence having at least 95% identity to SEQ ID NO: 136 and a light chain variable region comprising an amino acid sequence having at least 95% identity to SEQ ID NO: 313. In embodiments, the disclosure provides an antibody or antigen binding fragment thereof comprising: a heavy chain variable region comprising an amino acid sequence having at least 95% identity to SEQ ID NO: 137 and a light chain variable region comprising an amino acid sequence having at least 95% identity to SEQ ID NO: 314. In embodiments, the disclosure provides an antibody or antigen binding fragment thereof comprising: a heavy chain variable region comprising an amino acid sequence having at least 95% identity to SEQ ID NO: 149 and a light chain variable region comprising an amino acid sequence having at least 95% identity to SEQ ID NO: 326. In embodiments, the disclosure provides an antibody or antigen binding fragment thereof comprising: a heavy chain variable region comprising an amino acid sequence having at least 95% identity to SEQ ID NO: 156 and a light chain variable region comprising an amino acid sequence having at least 95% identity to SEQ ID NO: 333. In embodiments, the disclosure provides an antibody or antigen binding fragment thereof comprising: a heavy chain variable region comprising an amino acid sequence having at least 95% identity to SEQ ID NO: 158 and a light chain variable region comprising an amino acid sequence having at least 95% identity to SEQ ID NO: 335. In embodiments, the disclosure provides an antibody or antigen binding fragment thereof comprising: a heavy chain variable region comprising an amino acid sequence having at least 95% identity to SEQ ID NO: 143 and a light chain variable region comprising an amino acid sequence having at least 95% identity to SEQ ID NO: 320.

In embodiments, the disclosure provides an antibody or antigen binding fragment thereof comprising: a heavy chain variable region comprising an amino acid sequence having at least 97% identity to SEQ ID NO: 114 and a light chain variable region comprising an amino acid sequence having at least 97% identity to SEQ ID NO: 291. In embodiments, the disclosure provides an antibody or antigen binding fragment thereof comprising: a heavy chain variable region comprising an amino acid sequence having at least 97% identity to SEQ ID NO: 115 and a light chain variable region comprising an amino acid sequence having at least 97% identity to SEQ ID NO: 292. In embodiments, the disclosure provides an antibody or antigen binding fragment thereof comprising: a heavy chain variable region comprising an amino acid sequence having at least 97% identity to SEQ ID NO: 116 and a light chain variable region comprising an amino acid sequence having at least 97% identity to SEQ ID NO: 293. In embodiments, the disclosure provides an antibody or antigen binding fragment thereof comprising: a heavy chain variable region comprising an amino acid sequence having at least 97% identity to SEQ ID NO: 117 and a light chain variable region comprising an amino acid sequence having at least 97% identity to SEQ ID NO: 294. In embodiments, the disclosure provides an antibody or antigen binding fragment thereof comprising: a heavy chain variable region comprising an amino acid sequence having at least 97% identity to SEQ ID NO: 118 and a light chain variable region comprising an amino acid sequence having at least 97% identity to SEQ ID NO: 295. In embodiments, the disclosure provides an antibody or antigen binding fragment thereof comprising: a heavy chain variable region comprising an amino acid sequence having at least 97% identity to SEQ ID NO: 119 and a light chain variable region comprising an amino acid sequence having at least 97% identity to SEQ ID NO: 296. In embodiments, the disclosure provides an antibody or antigen binding fragment thereof comprising: a heavy chain variable region comprising an amino acid sequence having at least 97% identity to SEQ ID NO: 120 and a light chain variable region comprising an amino acid sequence having at least 97% identity to SEQ ID NO: 297. In embodiments, the disclosure provides an antibody or antigen binding fragment thereof comprising: a heavy chain variable region comprising an amino acid sequence having at least 97% identity to SEQ ID NO: 121 and a light chain variable region comprising an amino acid sequence having at least 97% identity to SEQ ID NO: 298. In embodiments, the disclosure provides an antibody or antigen binding fragment thereof comprising: a heavy chain variable region comprising an amino acid sequence having at least 97% identity to SEQ ID NO: 122 and a light chain variable region comprising an amino acid sequence having at least 97% identity to SEQ ID NO: 299. In embodiments, the disclosure provides an antibody or antigen binding fragment thereof comprising: a heavy chain variable region comprising an amino acid sequence having at least 97% identity to SEQ ID NO: 123 and a light chain variable region comprising an amino acid sequence having at least 97% identity to SEQ ID NO: 300. In embodiments, the disclosure provides an antibody or antigen binding fragment thereof comprising: a heavy chain variable region comprising an amino acid sequence having at least 97% identity to SEQ ID NO: 124 and a light chain variable region comprising an amino acid sequence having at least 97% identity to SEQ ID NO: 301. In embodiments, the disclosure provides an antibody or antigen binding fragment thereof comprising: a heavy chain variable region comprising an amino acid sequence having at least 97% identity to SEQ ID NO: 125 and a light chain variable region comprising an amino acid sequence having at least 97% identity to SEQ ID NO: 302. In embodiments, the disclosure provides an antibody or antigen binding fragment thereof comprising: a heavy chain variable region comprising an amino acid sequence having at least 97% identity to SEQ ID NO: 126 and a light chain variable region comprising an amino acid sequence having at least 97% identity to SEQ ID NO: 303. In embodiments, the disclosure provides an antibody or antigen binding fragment thereof comprising: a heavy chain variable region comprising an amino acid sequence having at least 97% identity to SEQ ID NO: 127 and a light chain variable region comprising an amino acid sequence having at least 97% identity to SEQ ID NO: 304. In embodiments, the disclosure provides an antibody or antigen binding fragment thereof comprising: a heavy chain variable region comprising an amino acid sequence having at least 97% identity to SEQ ID NO: 128 and a light chain variable region comprising an amino acid sequence having at least 97% identity to SEQ ID NO: 305. In embodiments, the disclosure provides an antibody or antigen binding fragment thereof comprising: a heavy chain variable region comprising an amino acid sequence having at least 97% identity to SEQ ID NO: 129 and a light chain variable region comprising an amino acid sequence having at least 97% identity to SEQ ID NO: 306. In embodiments, the disclosure provides an antibody or antigen binding fragment thereof comprising: a heavy chain variable region comprising an amino acid sequence having at least 97% identity to SEQ ID NO: 130 and a light chain variable region comprising an amino acid sequence having at least 97% identity to SEQ ID NO: 307. In embodiments, the disclosure provides an antibody or antigen binding fragment thereof comprising: a heavy chain variable region comprising an amino acid sequence having at least 97% identity to SEQ ID NO: 131 and a light chain variable region comprising an amino acid sequence having at least 97% identity to SEQ ID NO: 308. In embodiments, the disclosure provides an antibody or antigen binding fragment thereof comprising: a heavy chain variable region comprising an amino acid sequence having at least 97% identity to SEQ ID NO: 132 and a light chain variable region comprising an amino acid sequence having at least 97% identity to SEQ ID NO: 309. In embodiments, the disclosure provides an antibody or antigen binding fragment thereof comprising: a heavy chain variable region comprising an amino acid sequence having at least 97% identity to SEQ ID NO: 133 and a light chain variable region comprising an amino acid sequence having at least 97% identity to SEQ ID NO: 310. In embodiments, the disclosure provides an antibody or antigen binding fragment thereof comprising: a heavy chain variable region comprising an amino acid sequence having at least 97% identity to SEQ ID NO: 134 and a light chain variable region comprising an amino acid sequence having at least 97% identity to SEQ ID NO: 311. In embodiments, the disclosure provides an antibody or antigen binding fragment thereof comprising: a heavy chain variable region comprising an amino acid sequence having at least 97% identity to SEQ ID NO: 135 and a light chain variable region comprising an amino acid sequence having at least 97% identity to SEQ ID NO: 312. In embodiments, the disclosure provides an antibody or antigen binding fragment thereof comprising: a heavy chain variable region comprising an amino acid sequence having at least 97% identity to SEQ ID NO: 136 and a light chain variable region comprising an amino acid sequence having at least 97% identity to SEQ ID NO: 313. In embodiments, the disclosure provides an antibody or antigen binding fragment thereof comprising: a heavy chain variable region comprising an amino acid sequence having at least 97% identity to SEQ ID NO: 137 and a light chain variable region comprising an amino acid sequence having at least 97% identity to SEQ ID NO: 314. In embodiments, the disclosure provides an antibody or antigen binding fragment thereof comprising: a heavy chain variable region comprising an amino acid sequence having at least 97% identity to SEQ ID NO: 149 and a light chain variable region comprising an amino acid sequence having at least 97% identity to SEQ ID NO: 326. In embodiments, the disclosure provides an antibody or antigen binding fragment thereof comprising: a heavy chain variable region comprising an amino acid sequence having at least 97% identity to SEQ ID NO: 156 and a light chain variable region comprising an amino acid sequence having at least 97% identity to SEQ ID NO: 333. In embodiments, the disclosure provides an antibody or antigen binding fragment thereof comprising: a heavy chain variable region comprising an amino acid sequence having at least 97% identity to SEQ ID NO: 158 and a light chain variable region comprising an amino acid sequence having at least 97% identity to SEQ ID NO: 335. In embodiments, the disclosure provides an antibody or antigen binding fragment thereof comprising: a heavy chain variable region comprising an amino acid sequence having at least 97% identity to SEQ ID NO: 143 and a light chain variable region comprising an amino acid sequence having at least 97% identity to SEQ ID NO: 320.

In embodiments, the disclosure provides an antibody or antigen binding fragment thereof comprising: a heavy chain variable region comprising an amino acid sequence having at least 99% identity to SEQ ID NO: 114 and a light chain variable region comprising an amino acid sequence having at least 99% identity to SEQ ID NO: 291. In embodiments, the disclosure provides an antibody or antigen binding fragment thereof comprising: a heavy chain variable region comprising an amino acid sequence having at least 99% identity to SEQ ID NO: 115 and a light chain variable region comprising an amino acid sequence having at least 99% identity to SEQ ID NO: 292. In embodiments, the disclosure provides an antibody or antigen binding fragment thereof comprising: a heavy chain variable region comprising an amino acid sequence having at least 99% identity to SEQ ID NO: 116 and a light chain variable region comprising an amino acid sequence having at least 99% identity to SEQ ID NO: 293. In embodiments, the disclosure provides an antibody or antigen binding fragment thereof comprising: a heavy chain variable region comprising an amino acid sequence having at least 99% identity to SEQ ID NO: 117 and a light chain variable region comprising an amino acid sequence having at least 99% identity to SEQ ID NO: 294. In embodiments, the disclosure provides an antibody or antigen binding fragment thereof comprising: a heavy chain variable region comprising an amino acid sequence having at least 99% identity to SEQ ID NO: 118 and a light chain variable region comprising an amino acid sequence having at least 99% identity to SEQ ID NO: 295. In embodiments, the disclosure provides an antibody or antigen binding fragment thereof comprising: a heavy chain variable region comprising an amino acid sequence having at least 99% identity to SEQ ID NO: 119 and a light chain variable region comprising an amino acid sequence having at least 99% identity to SEQ ID NO: 296. In embodiments, the disclosure provides an antibody or antigen binding fragment thereof comprising: a heavy chain variable region comprising an amino acid sequence having at least 99% identity to SEQ ID NO: 120 and a light chain variable region comprising an amino acid sequence having at least 99% identity to SEQ ID NO: 297. In embodiments, the disclosure provides an antibody or antigen binding fragment thereof comprising: a heavy chain variable region comprising an amino acid sequence having at least 99% identity to SEQ ID NO: 121 and a light chain variable region comprising an amino acid sequence having at least 99% identity to SEQ ID NO: 298. In embodiments, the disclosure provides an antibody or antigen binding fragment thereof comprising: a heavy chain variable region comprising an amino acid sequence having at least 99% identity to SEQ ID NO: 122 and a light chain variable region comprising an amino acid sequence having at least 99% identity to SEQ ID NO: 299. In embodiments, the disclosure provides an antibody or antigen binding fragment thereof comprising: a heavy chain variable region comprising an amino acid sequence having at least 99% identity to SEQ ID NO: 123 and a light chain variable region comprising an amino acid sequence having at least 99% identity to SEQ ID NO: 300. In embodiments, the disclosure provides an antibody or antigen binding fragment thereof comprising: a heavy chain variable region comprising an amino acid sequence having at least 99% identity to SEQ ID NO: 124 and a light chain variable region comprising an amino acid sequence having at least 99% identity to SEQ ID NO: 301. In embodiments, the disclosure provides an antibody or antigen binding fragment thereof comprising: a heavy chain variable region comprising an amino acid sequence having at least 99% identity to SEQ ID NO: 125 and a light chain variable region comprising an amino acid sequence having at least 99% identity to SEQ ID NO: 302. In embodiments, the disclosure provides an antibody or antigen binding fragment thereof comprising: a heavy chain variable region comprising an amino acid sequence having at least 99% identity to SEQ ID NO: 126 and a light chain variable region comprising an amino acid sequence having at least 99% identity to SEQ ID NO: 303. In embodiments, the disclosure provides an antibody or antigen binding fragment thereof comprising: a heavy chain variable region comprising an amino acid sequence having at least 99% identity to SEQ ID NO: 127 and a light chain variable region comprising an amino acid sequence having at least 99% identity to SEQ ID NO: 304. In embodiments, the disclosure provides an antibody or antigen binding fragment thereof comprising: a heavy chain variable region comprising an amino acid sequence having at least 99% identity to SEQ ID NO: 128 and a light chain variable region comprising an amino acid sequence having at least 99% identity to SEQ ID NO: 305. In embodiments, the disclosure provides an antibody or antigen binding fragment thereof comprising: a heavy chain variable region comprising an amino acid sequence having at least 99% identity to SEQ ID NO: 129 and a light chain variable region comprising an amino acid sequence having at least 99% identity to SEQ ID NO: 306. In embodiments, the disclosure provides an antibody or antigen binding fragment thereof comprising: a heavy chain variable region comprising an amino acid sequence having at least 99% identity to SEQ ID NO: 130 and a light chain variable region comprising an amino acid sequence having at least 99% identity to SEQ ID NO: 307. In embodiments, the disclosure provides an antibody or antigen binding fragment thereof comprising: a heavy chain variable region comprising an amino acid sequence having at least 99% identity to SEQ ID NO: 131 and a light chain variable region comprising an amino acid sequence having at least 99% identity to SEQ ID NO: 308. In embodiments, the disclosure provides an antibody or antigen binding fragment thereof comprising: a heavy chain variable region comprising an amino acid sequence having at least 99% identity to SEQ ID NO: 132 and a light chain variable region comprising an amino acid sequence having at least 99% identity to SEQ ID NO: 309. In embodiments, the disclosure provides an antibody or antigen binding fragment thereof comprising: a heavy chain variable region comprising an amino acid sequence having at least 99% identity to SEQ ID NO: 133 and a light chain variable region comprising an amino acid sequence having at least 99% identity to SEQ ID NO: 310. In embodiments, the disclosure provides an antibody or antigen binding fragment thereof comprising: a heavy chain variable region comprising an amino acid sequence having at least 99% identity to SEQ ID NO: 134 and a light chain variable region comprising an amino acid sequence having at least 99% identity to SEQ ID NO: 311. In embodiments, the disclosure provides an antibody or antigen binding fragment thereof comprising: a heavy chain variable region comprising an amino acid sequence having at least 99% identity to SEQ ID NO: 135 and a light chain variable region comprising an amino acid sequence having at least 99% identity to SEQ ID NO: 312. In embodiments, the disclosure provides an antibody or antigen binding fragment thereof comprising: a heavy chain variable region comprising an amino acid sequence having at least 99% identity to SEQ ID NO: 136 and a light chain variable region comprising an amino acid sequence having at least 99% identity to SEQ ID NO: 313. In embodiments, the disclosure provides an antibody or antigen binding fragment thereof comprising: a heavy chain variable region comprising an amino acid sequence having at least 99% identity to SEQ ID NO: 137 and a light chain variable region comprising an amino acid sequence having at least 99% identity to SEQ ID NO: 314. In embodiments, the disclosure provides an antibody or antigen binding fragment thereof comprising: a heavy chain variable region comprising an amino acid sequence having at least 99% identity to SEQ ID NO: 149 and a light chain variable region comprising an amino acid sequence having at least 99% identity to SEQ ID NO: 326. In embodiments, the disclosure provides an antibody or antigen binding fragment thereof comprising: a heavy chain variable region comprising an amino acid sequence having at least 99% identity to SEQ ID NO: 156 and a light chain variable region comprising an amino acid sequence having at least 99% identity to SEQ ID NO: 333. In embodiments, the disclosure provides an antibody or antigen binding fragment thereof comprising: a heavy chain variable region comprising an amino acid sequence having at least 99% identity to SEQ ID NO: 158 and a light chain variable region comprising an amino acid sequence having at least 99% identity to SEQ ID NO: 335. In embodiments, the disclosure provides an antibody or antigen binding fragment thereof comprising: a heavy chain variable region comprising an amino acid sequence having at least 99% identity to SEQ ID NO: 143 and a light chain variable region comprising an amino acid sequence having at least 99% identity to SEQ ID NO: 320.

The SCF248 isoform of SCF includes exon 6, which comprises a cleavage site between two alanine residues (amino acids 16 and 17 of SEQ ID NO: 473, which provides the amino acid sequence of exon 6). Previous anti-SCF antibodies were generated by immunizing mice with a peptide spanning exon 6 and part of Exon 7 (see, e.g., U.S. Pat. No. 8,911,729, which is hereby incorporated by reference in its entirety for all purposes). Since SCF220 is associated with homeostatic activities, any cross-reactivity with SCF220 would be detrimental as it would result in various off-target effects in subjects. Advantageously, the antibodies provided in the present disclosure bind to SCF248 with very high specificity. In some embodiments, the antibodies provided herein are specific for SCF248 and do not bind to SCF220. Thus, the antibodies provided herein are capable of specifically inhibiting the interaction between SCF248 and c-Kit that induces and perpetuates chronic inflammatory responses and fibrosis. Moreover, the antibodies provided herein are capable of specifically inducing the internalization of SCF and thereby reducing the interaction between SCF248 and c-Kit. Accordingly, in some embodiments the present disclosure provides antibodies that are specific for SCF248 and are safe and effective in various inflammatory and fibrotic diseases discussed herein and known in the art.

For preparation of monoclonal antibodies, any technique that provides for the production of antibody molecules by continuous cell lines in culture may be used (see e.g., Harlow and Lane, Antibodies: A Laboratory Manual, Cold Spring Harbor Laboratory Press, Cold Spring Harbor, N.Y.). These include, but are not limited to, the hybridoma technique originally developed by Kohler and Milstein and the trioma technique, the human B-cell hybridoma technique (See, e.g., Kozbor et al., Immunol. Today, 4:72 (1983)), and the EBV-hybridoma technique to produce human monoclonal antibodies (Cole et al., in Monoclonal Antibodies and Cancer Therapy, Alan R. Liss, Inc., pp. 77-96 (1985)). Alternatively, the antibodies may be made by recombinant DNA methods. In some embodiments, antibodies in accordance with the present disclosure may be made by isolating monoclonal antibodies from phage display libraries using the techniques described, for example, in Clackson et al., Nature 352:624-28 (1991) and Marks et al., J. Mol. Biol. 222(3):581-97 (1991). In some embodiments, the antibodies are fully human antibodies constructed by combining Fv clone variable domain sequence(s) selected from human-derived phage display or yeast display libraries with known human constant domain sequence(s).

In some embodiments provided herein, the antibodies are prepared from a hybridoma. Using the hybridoma method, a mouse, hamster, or other appropriate host animal, is immunized by injecting an immunizing peptide to elicit the production by lymphocytes of antibodies that will specifically bind to an immunizing antigen. Alternatively, lymphocytes can be immunized in vitro. Following immunization, the lymphocytes are isolated and fused with a suitable myeloma cell line using, for example, polyethylene glycol, to form hybridoma cells that can then be selected away from unfused lymphocytes and myeloma cells. Hybridomas that produce monoclonal antibodies directed specifically against a chosen antigen as determined by immunoprecipitation, immunoblotting, or by an in vitro binding assay such as radioimmunoassay (RIA) or enzyme-linked immunosorbent assay (ELISA) can then be propagated in vitro (e.g., in culture) using standard methods (Goding, Monoclonal Antibodies: Principles and Practice, Academic Press, 1986) or in vivo as ascites tumors in an animal. The monoclonal antibodies can then be purified from the culture medium or ascites fluid as described for polyclonal antibodies above.

In some embodiments, the antibodies provided herein are generated using the murine hybridoma system. Hybridoma production in the mouse is a well-established procedure. Immunization protocols and techniques for isolation of immunized splenocytes for fusion are known in the art. Fusion partners (e.g., murine myeloma cells) and fusion procedures are also known. Embodiments of the technology herein provide antibodies (e.g., monoclonal antibodies) produced from a hybridoma prepared by immunizing mice with a peptide that is a portion or fragment of the SCF protein.

In some embodiments, the antibodies specific for SCF248 provided herein are generated by immunizing mice with a peptide having an amino acid sequence that is largely or exclusively within exon 6. For example, the immunizing peptide comprises any stretch of 5 or more amino acids within SEQ ID NO: 473. As another example, the immunizing peptide comprises any stretch of 5 or more amino acids beginning at amino acid position 20 of SEQ ID NO: 470. As another example, the immunizing peptide comprises a stretch of 5 or more amino acids beginning at amino acid position 20 of SEQ ID NO: 470 and ending at any one of positions 25 to 38 of SEQ ID NO: 470. Thus, in some embodiments, the immunizing peptide comprises the amino acid sequence of exon 6 after the cleavage site, and is either fully contained within exon 6 or comprises only 1, 2, 3, 4, or 5 amino acids of exon 7. In some embodiments, the immunizing peptide comprises or consists of SEQ ID NO: 474. In some embodiments, the immunizing peptide comprises any of the peptides provided herein or conservative variants thereof. Conservative variants may comprise 1, 2, 3, 4, or 5 amino acid substitutions or deletions, or a combination thereof. As provided above, in some embodiments, the antibodies generated using the immunizing peptides provided herein have an epitope that falls entirely or largely within exon 6. By “largely within” it is meant that at least 75%, at least 80%, at least 85%, at least 90%, or at least 95% of the peptide falls within exon 6. In some embodiments, the epitope begins at the cleavage site of exon 6 (i.e., between the alanines at amino acid positions 19 and 20 of SEQ ID NO: 470 and extends to the end of exon 6. In some embodiments, the epitope begins at the cleavage site of exon 6 and extends to the 1^{st}, 2^{nd}, 3^{rd}, 4^{th}, or 5^{th} n-terminal amino acid of the transmembrane domain. In some embodiments, the epitope comprises or consists of SEQ ID NO: 471. In some embodiments, the antibody referred to herein as 5H10 (including the murine, chimeric, and humanized 5H10 antibodies) binds to an epitope of SCF comprising or consisting of SEQ ID NO: 471.

In some embodiments, the methods provided herein were used to generate antibodies referred to herein as 5H10 variants. Antibody 5H10 advantageously binds SCF248 with high specificity and does not bind SCF220. The amino acid sequences of the murine parent antibody 5H10, as well as humanized variants thereof, are provided herein (see, Tables 1 and 2). In some embodiments, provided herein are methods of improving the affinity of the humanized 5H10 variants for SCF. In some embodiments, the humanized 5H10 variants are subjected to affinity maturation using the method described in Example 2. In some embodiments, the affinity of the humanized 5H10 variants for SCF is improved by generating combinatorial libraries using phage, yeast, or ribosome display technologies and selecting for antibodies or fragments thereof with improved affinity. In some embodiments, each member of the combinatorial library comprises a 5H10 parental sequence (e.g., the murine parental sequence or a humanized variant thereof) with one or more mutations in hCDR1, hCDR2, hCDR3, lCDR1, lCDR2, or lCDR3. Exemplary antibodies with significantly higher affinity for SCF relative to the humanized 5H10 parental antibody are provided in Tables 1 and 2.

In one embodiment, the present invention provides bispecific or multispecific antibodies specific for SCF and at least one other antigen or epitope. The anti-SCF antibodies and fragments thereof provided herein may be tested for binding to SCF using the binding assays provided herein, or any other binding assay known in the art.

Unless otherwise stated, the practice of the present invention employs conventional molecular biology, cell biology, biochemistry, and immunology techniques that are well known in the art and described, for example, in Methods in Molecular Biology, Humana Press; Molecular Cloning: A Laboratory Manual, second edition (Sambrook et al., 1989), Current Protocols in Immunology (J. E. Coligan et al., eds., 1991); Immunobiology (C. A. Janeway and P. Travers, 1997); Antibodies (P. Finch, 1997); Antibodies: a practical approach (D. Catty., ed., IRL Press, 1988-1989); Monoclonal antibodies: a practical approach (P. Shepherd and C. Dean, eds., Oxford University Press, 2000); Phage display: a laboratory manual (C. Barbas III et al, Cold Spring Harbor Laboratory Press, 2001); and Using antibodies: a laboratory manual (E. Harlow and D. Lane (Cold Spring Harbor Laboratory Press, 1999).

### Methods of Treatment

In one aspect, the present disclosure provides methods for treating and/or preventing any disease or condition associated with immune cell migration, activation, and/or proliferation via interaction of SCF248 with c-Kit on immune cells. Thus, in some embodiments, the present disclosure provides methods for inhibiting or preventing activation of immune cells; as well as reducing or preventing the accumulation of immune cells within organs or tissues, thereby treating or preventing various diseases and disorders that involve inflammation. In some embodiments, the immune cells are selected from the group consisting of mast cells, innate lymphoid cells (ILCs, such as ILC2 or ILC3 cells), and eosinophils.

As used herein, the terms “treatment” or “treating” refers to both therapeutic treatment and prophylactic or preventive measures. Subjects in need of treatment include those subjects that already have the disease or condition, as well as those that may develop the disease or condition and in whom the object is to prevent, delay, or diminish the disease or condition. As used herein, the term “subject” denotes a mammal, such as a rodent, a feline, a canine, and a primate. Preferably, a subject according to the invention is a human. The term “therapeutically effective amount,” as used herein, refers to the amount of a compound or composition that is necessary to provide a therapeutic and/or preventative benefit to the subject.

In one aspect the present invention provides methods for treating a subject for an inflammatory disease, a fibrotic disease, and/or a tissue remodeling disease. In some embodiments, the inflammatory disease is a chronic inflammatory disease.

Chronic inflammatory, fibrotic, and tissue remodeling diseases include diseases of the lung, kidney, liver, heart, skin, connective tissue, and other tissues. Exemplary inflammatory, fibrotic or tissue remodeling diseases include, without limitation, pulmonary fibrosis (e.g., idiopathic pulmonary fibrosis (IPF), scleroderma lung fibrosis, scleroderma-related interstitial lung disease (SSc-ILD), pulmonary fibrosis associated with a lung infection or pneumonia, pulmonary fibrosis associated with systemic lupus erythematosus and/or rheumatoid arthritis, sarcoidosis), chronic obstructive pulmonary disease (COPD), acute respiratory distress syndrome (ARDS), cystic fibrosis, peribronchial fibrosis, bleomycin lung, hypersensitivity pneumonitis, asthma, fibrothorax, mediastinal fibrosis, chronic rhinosinusitis, urticaria (e.g., chronic spontaneous urticaria), atopic dermatitis, dermatomyositis, nodular subepidermal fibrosis, scleroderma, keloid, renal fibrosis, chronic kidney disease, glomerulonephritis, chronic renal allograft rejection, nephropathy (e.g., IgA nephropathy, focal segmental glomerulosclerosis, rapidly progressive glomerulonephritis, crescentic glomerulonephritis, lupus nephritis, hypertensive nephropathy, or diabetic nephropathy), non-alcoholic steatohepatitis (NASH), liver cirrhosis, hepatic fibrosis, primary sclerosing cholangitis, primary biliary cirrhosis, fibromyalgia, gingival fibrosis, radiation-induced fibrosis, eosinophilic esophagitis, inflammatory bowel disease (IBD), arthrofibrosis, and atrial fibrosis, endomyocardial fibrosis, parenchymal fibrosis, fibrous histocytoma, or glial scarring.

In some embodiments, the antibodies and fragments thereof disclosed herein may be administered to the subject by at least one route selected from parenteral, subcutaneous, intramuscular, intravenous, intrarticular, intrabronchial, intraabdominal, intracapsular, intracartilaginous, intracavitary, intracelial, intracerebellar, intracerebroventricular, intracolic, intracervical, intragastric, intrahepatic, intramyocardial, intraosteal, intrapelvic, intrapericardiac, intraperitoneal, intrapleural, intraprostatic, intrapulmonary, intrarectal, intrarenal, intraretinal, intraspinal, intrasynovial, intrathoracic, intratympanic, intrauterine, intravesical, intravitreal, bolus, subconjunctival, oral, vaginal, rectal, buccal, sublingual, intranasal, intratumoral, and transdermal.

In embodiments, the antibodies and fragments thereof disclosed herein may be administered to a subject in need thereof in combination with one or more additional therapy. The one or more additional therapy may be a procedure such as a surgical procedure, or may be a therapeutic agent, such as an agent designed to mitigate or reduce symptoms of a disease or disorder associated with fibrosis and/or inflammation.

In embodiments, the methods provided herein reduce inflammation. In embodiments, the methods provided herein reduce expression of one or more cytokines. For example, the methods provided herein may reduce expression of one or more interleukins, (e.g., IL-4, IL-19, IL-13, IL-25, IL-1, IL-6,), transforming growth factor beta (TGFβ), chemokine ligand 2 (CCL2), or tumor necrosis factor alpha (TNF-α). In embodiments, expression is measured as in Example 4. In embodiments, expression is measured as compared to a control. In embodiments, the control is cells that are not exposed to the antibody or variant. The 5H10 VK3/VH1 humanized antibody of Table 1 reduces expression of CCL2 and TGFβ. The table below shows expression of CCL2 and TGFβ in cells that were exposed to the 5H10 VK3/VH1 humanized antibody of Table 1. Expression is relative to a positive control that was not exposed to an antibody.

| | CCL2 | | TGFβ | |
|---|---|---|---|---|
| | Expression as | | Expression as | |
| | Percentage of | Standard | Percentage of | Standard |
| | Positive Control | error | Positive Control | error |
| 5H10 | 37 | 3.6 | 65.5 | 7.5 |
| VK3/VH1 | | | | |
| humanized | | | | |

In embodiments, provided herein are antibodies or variants thereof that as compared to a positive control, exhibit less than 37% expression of CCL2. In embodiments, provided herein are antibodies or variants thereof that as compared to a positive control, exhibit less than 65.5% expression of TGFβ. In embodiments, provided herein are antibodies that reduce CCL2 or TGFβ expression in a cell by at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, or at least about 95% more than the 5H10 VK3/VH1 humanized antibody of Table 1. In embodiments, CCL2 or TGFβ expression is measured compared to a control as in Example 4.

The present invention is further illustrated by reference to the following Examples. However, it should be noted that these Examples, like the embodiments described above, are illustrative and are not to be construed as restricting the scope of the invention in any way.

### EXAMPLES

The following examples are given for the purpose of illustrating various embodiments of the disclosure and are not meant to limit the present disclosure in any fashion. Changes therein and other uses which are encompassed within the spirit of the disclosure, as defined by the scope of the claims, will be recognized by those skilled in the art.

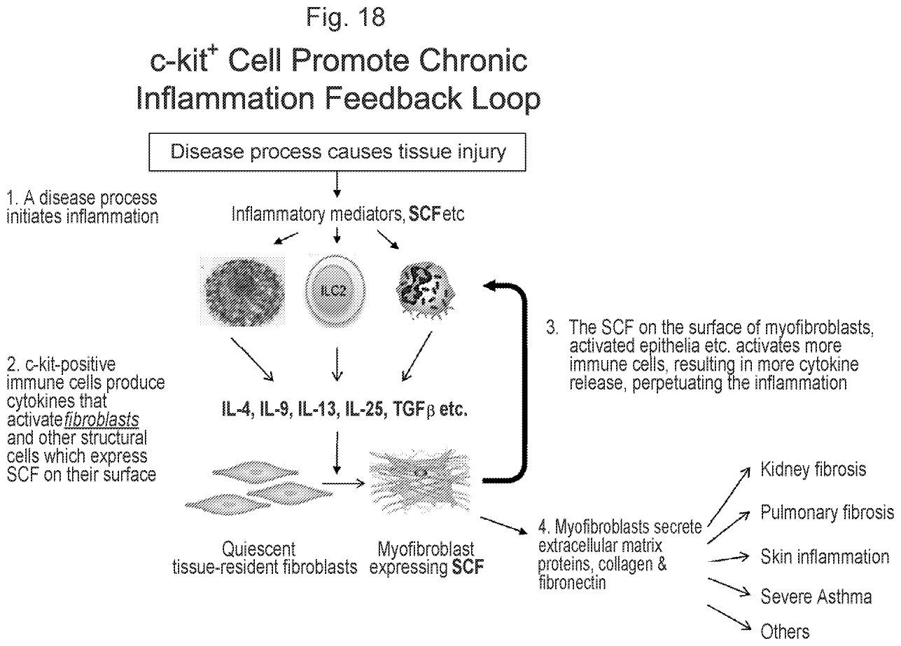

An overview of the tissue injury/disease process is summarized in FIG. 18. A disease process initiates inflammation. c-Kit+ immune cells produce cytokines that cause fibroblasts to change into activated myofibroblasts which express SCF248 on their surface. The expression of SCF248 on the surface of myofibroblasts and other cells activates more immune cells, resulting in cytokine release of IL-4, IL-9, IL-13, IL-25, TGFβ, and other cytokines, perpetuating inflammation. Myofibroblasts secrete extracellular matrix proteins, collagen, and fibronectin, leading to fibrosis and remodeling diseases such as pulmonary fibrosis, skin fibrosis, severe asthma, and other diseases.

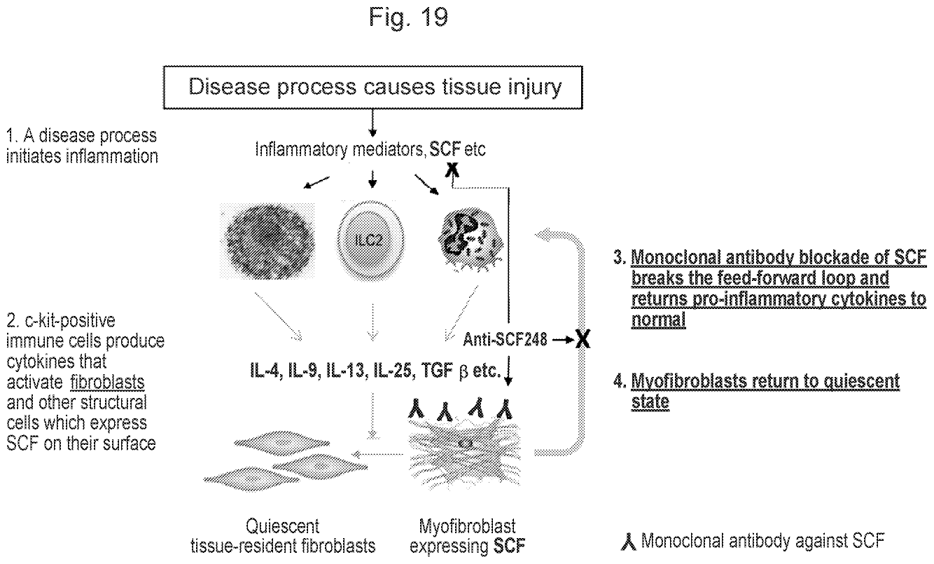

An exemplary mechanism of an antibody of the instant disclosure which targets SCF248 is summarized in FIG. 19.

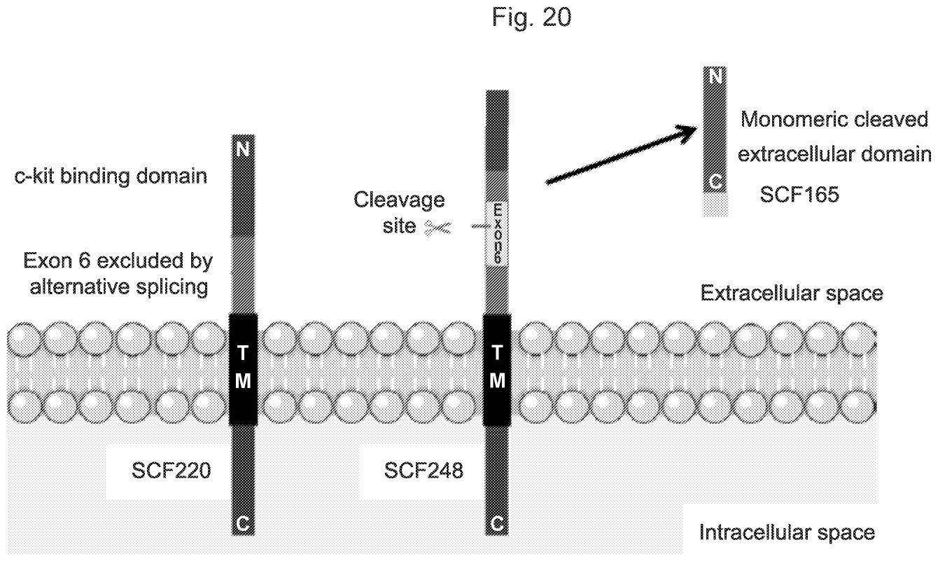

As provided above, SCF has two isoforms which result from alternative splicing: SCF248 and SCF220. SCF248 and SCF220 differ by exon 6. SCF220 is associated with homeostatic functions, and SCF248 is associated with inflammation and fibrosis. SCF248 activates immune cells during inflammation and is sometimes called “soluble SCF.” SCF248 is expressed on various cell types including myofibroblasts, activated epithelia, endothelia, macrophages, eosinophils, mast cells, and monocytes (FIG. 20). The SCF248 isoform results in cleavage of monomeric cleaved extracellular domain, called SCF165. The amino acid sequence of exon 6 is provided herein as SEQ ID NO: 473.

### Example 1: Affinity of Humanized 5H10 scFv (“VK3/VH1”)

5H10 was humanized by engrafting the complementarity determining regions (CDRs) on a human scaffold. The resultant humanized antibody was referred to as “VK3/VH1” or “parental antibody.”

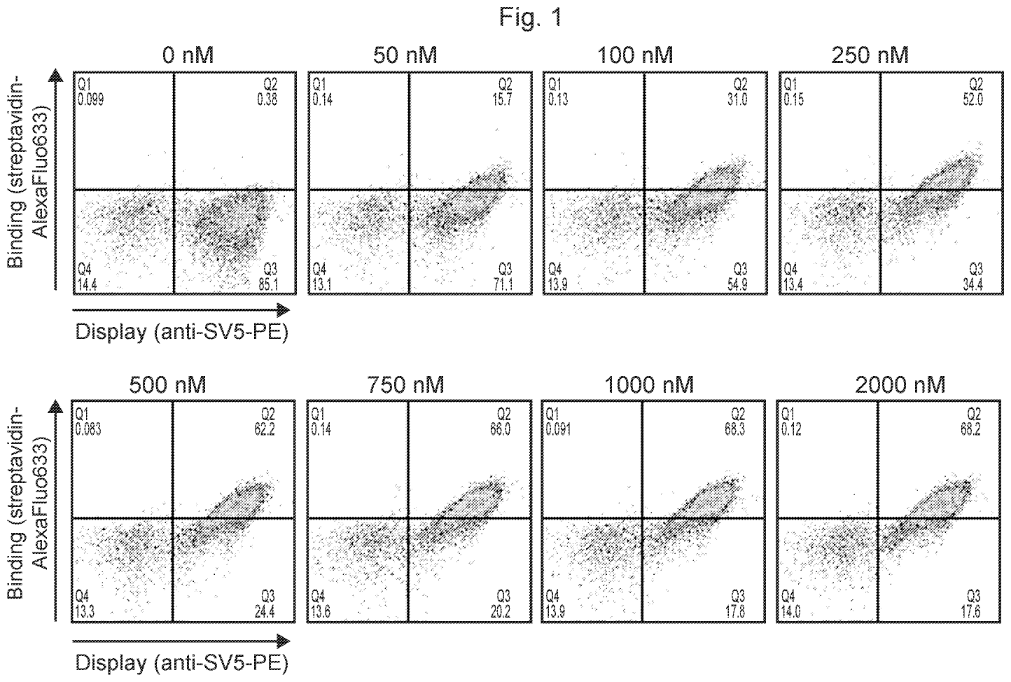

The VH and VL domains of the parental antibody were cloned into the pSYD yeast display vector and displayed as a single chain variable fragment (scFv) (VH-linker-VL-SV5tag) on the surface of yeast cells. The parental scFv contained from N-terminus to C-terminus a variable heavy domain, a linker, a variable light domain, and an SV5 tag. The yeast display vector carried a galactose inducible promoter, a secretion leader, a multicloning site and the Aga2 protein sequence for the C-terminal anchoring of the scFv molecule on the yeast surface [1]. Yeast display methods were conducted as described by Ferrara et al [2]. Briefly, cells were induced in induction media overnight at 20° C. 10⁵ induced cells were washed twice with wash buffer (PBS supplemented with 0.5% BSA) and incubated at room temperature with the biotinylated antigen diluted in PBS. To assess binding of the scFv, a C-terminal biotinylated peptide mapping on Exon6 of the SCF molecule was used (referred to herein as “PE9413”). PE9413 has an amino acid sequence of ASSLRNDSSSSNRKAKNPPGDS (SEQ ID NO: 479). The induced yeast population was stained with biotinylated PE9413 at different concentrations ranging from 50 nM to 2 μM (0 nM, 50 nM, 100 nM, 250 nM, 500 nM, 750 nM, 1000 nM, or 2000 nM). Volumes and incubation times were carefully adjusted according to parameters described in [3]. After the incubation with the target peptide, the yeast cells were washed twice with cold wash buffer and subsequently incubated at 4° C. for an additional 30 minutes with fluorescently labelled streptavidin (Streptavidin-AlexaFlu633), to detect binding of the biotinylated PE9413, and with the labelled anti-SV5 (anti-SV5-PE), to detect the scFv display levels on yeast cells. Following 2 washes with cold yeast wash buffer, the cells were resuspended in cold PBS and analyzed in the flow cytometer (FIG. 1).

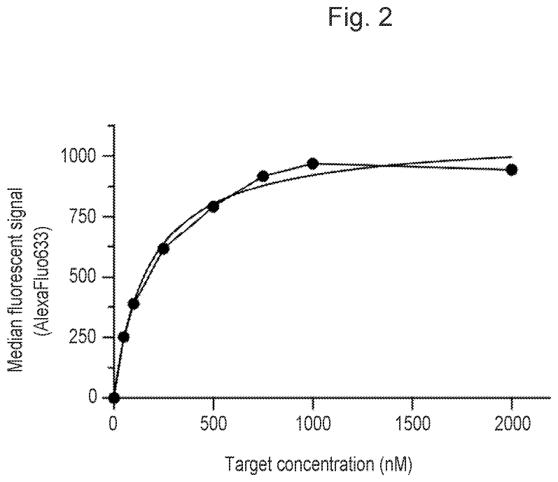

The median fluorescent signal of the binding population was plotted against the PE9413 target and used to estimate the affinity of the parental antibody for PE9413 (FIG. 2). The K_{D} for the parental antibody was 173.8 nM (R²=0.9922).

The parental antibody was also displayed on yeast cells as a scFv in alternate orientations, including as VL-linker-VH-SV5tag. Alternate vectors that allowed for N-terminal anchoring of the scFv molecule on the yeast surface via Aga2 were also explored. Similar affinity measurements were obtained.

### Example 2: Affinity Maturation of VK3/VH1 Antibody

The humanized anti-SCF 5H10 VK3/VH1 antibody (referred to as “parental” throughout this document) was subjected to affinity maturation by mutational scanning of its CDRs and yeast display screening of the mutants.

### Development of Mutational Scanning Libraries

Oligonucleotides (oligos) encoding the parental CDRs and oligonucleotides encoding the parental CDRS with single amino acid mutations at each CDR residue were designed and synthesized (Table A1).

TABLE A1
Kabat-annotated CDRs of 5H10 VK3/VH1 and
number of oligonucleotides used for
affinity maturation

| | | # oligos |
|---|---|---|
| | | (including |
| CDR | Amino acid sequence | parental) |
| HCDR1 | SYWMN (SEQ ID NO: 14) | 95 |
| HCDR2 | QIYPGDGDTHYNGKFKG (SEQ | 323 |
| | ID NO: 15) | |
| HCDR3 | SNWVGSY(SEQ ID NO: 16) | 133 |
| LCDR1 | KSSQSLLESDGKTYLN(SEQ | 304 |
| | ID NO: 17) | |
| LCDR2 | LVSRLDS (SEQ ID NO: 18) | 133 |
| LCDR3 | WQGTHLPQT (SEQ ID NO: | 171 |
| | 19) | |

The single amino acid mutations could be any of the twenty natural amino acids except for cysteine (e.g., 19 possible amino acid changes at each CDR position). The oligos were synthesized with parental framework flanking regions to facilitate the assembly of a complete scFv.

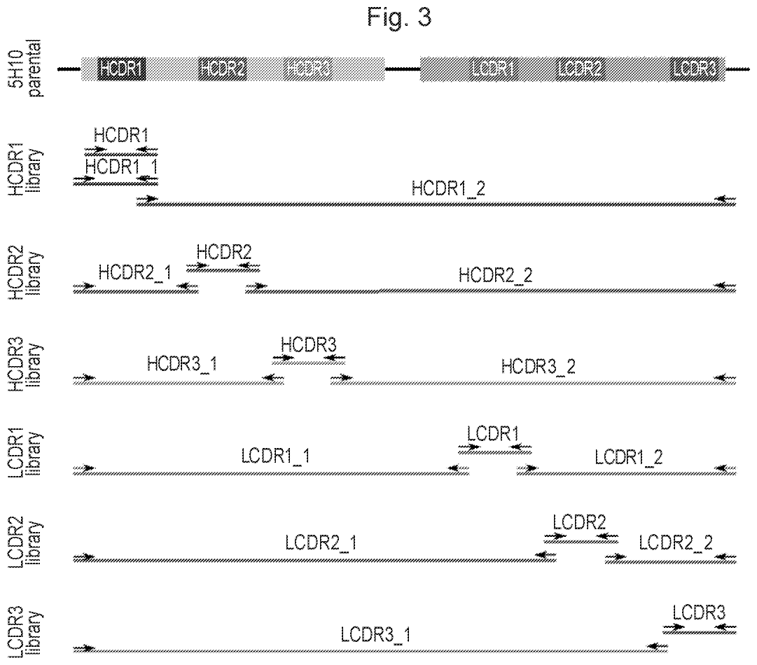

The oligonucleotides for each CDR were separately pooled to form six different collections of CDR sequences (also referred to as “mutational scanning libraries”). Each collection was synthesized by array synthesis and individually amplified from the pool with specific primers by polymerase chain reaction (PCR) using a high fidelity Q5 polymerase. The remaining regions for the reconstitution of the full-length scFv were amplified from the parental antibody gene and assembled with the CDRs by PCR. In each library, one of the parental CDRs was replaced by the designed mutational oligos as shown in FIG. 3 and the assembly products were cloned in the pSYD yeast display vector by in vivo homologous recombination [4]. The scFvs were assembled in the “VH-VL” orientation.

### Screening of Mutational Scanning Libraries

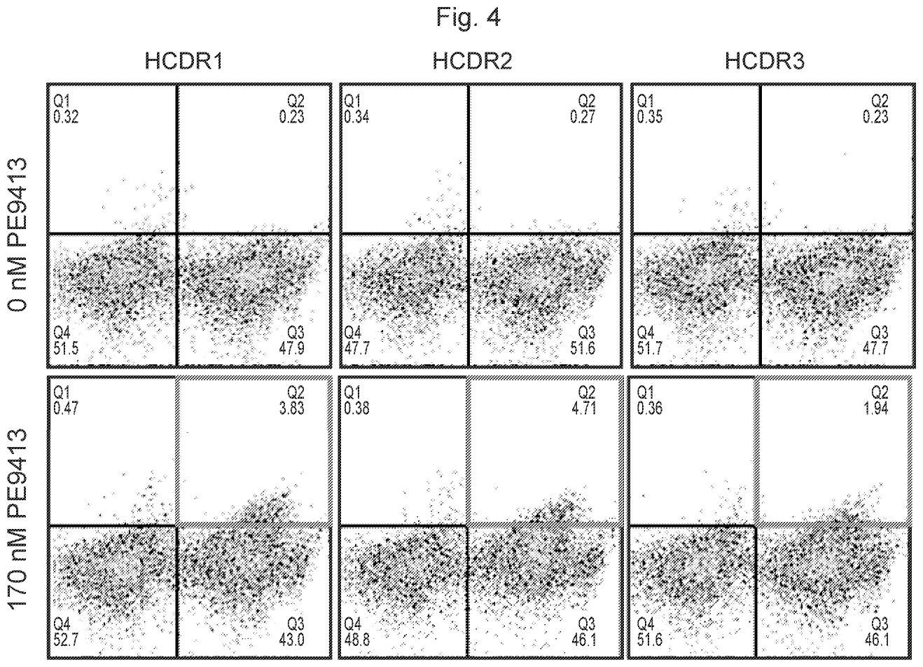

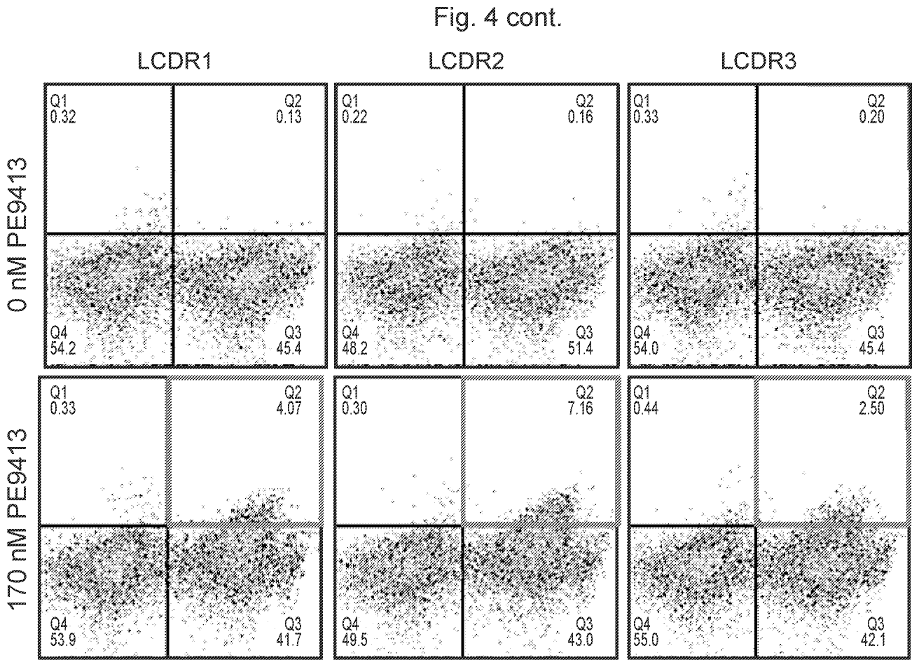

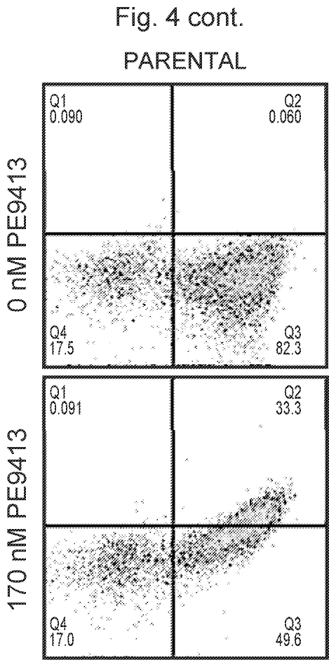

The 6 mutational scanning CDR libraries in yeast were subsequently induced and stained with the target PE9413 at a concentration of 170 nM. The yeast cells showing a fluorescent binding signal above background were sorted by Fluorescence-Activated Cell Sorting (FACS) and expanded for subsequent rounds of selection (FIG. 4).

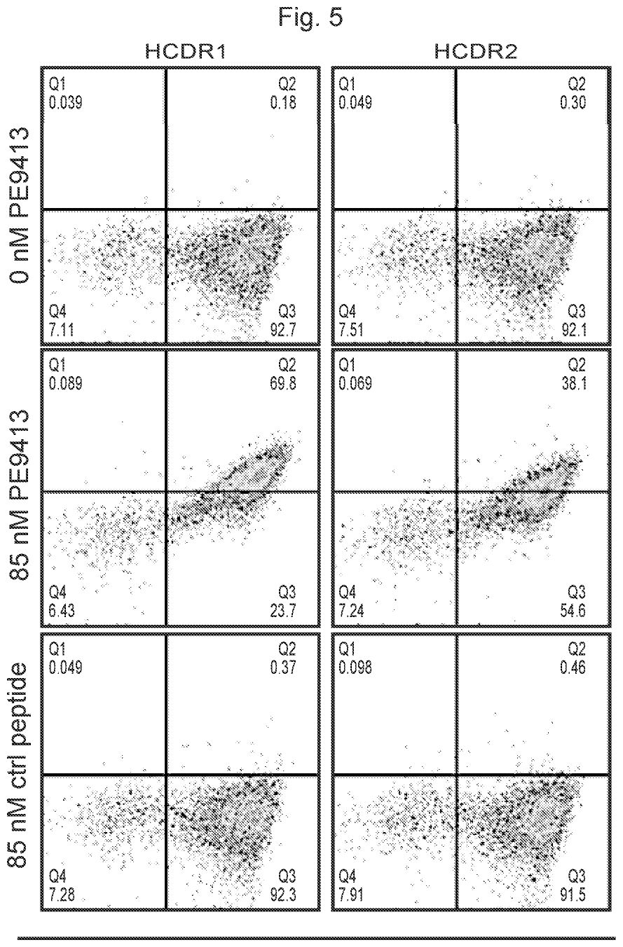

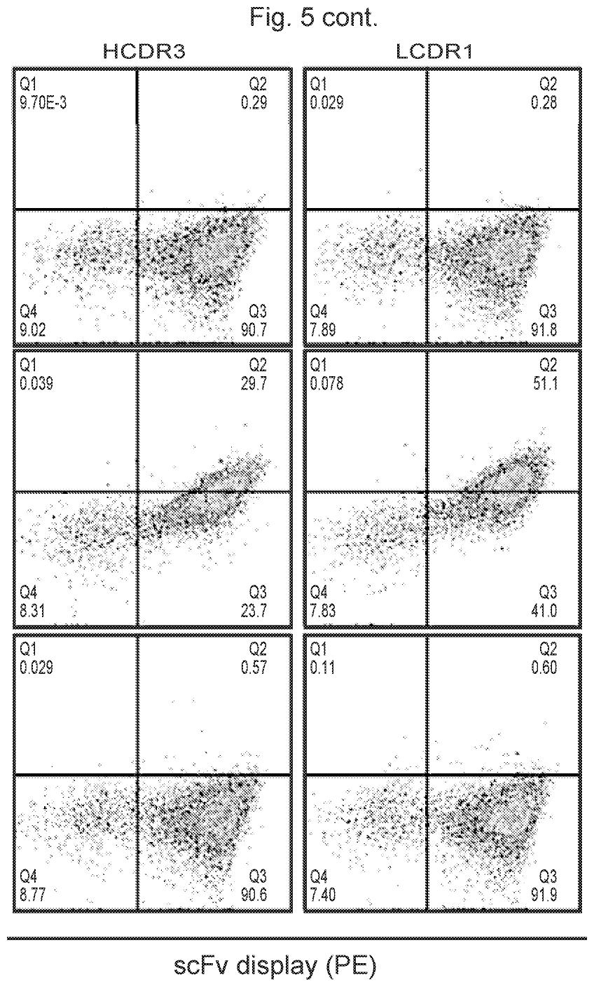

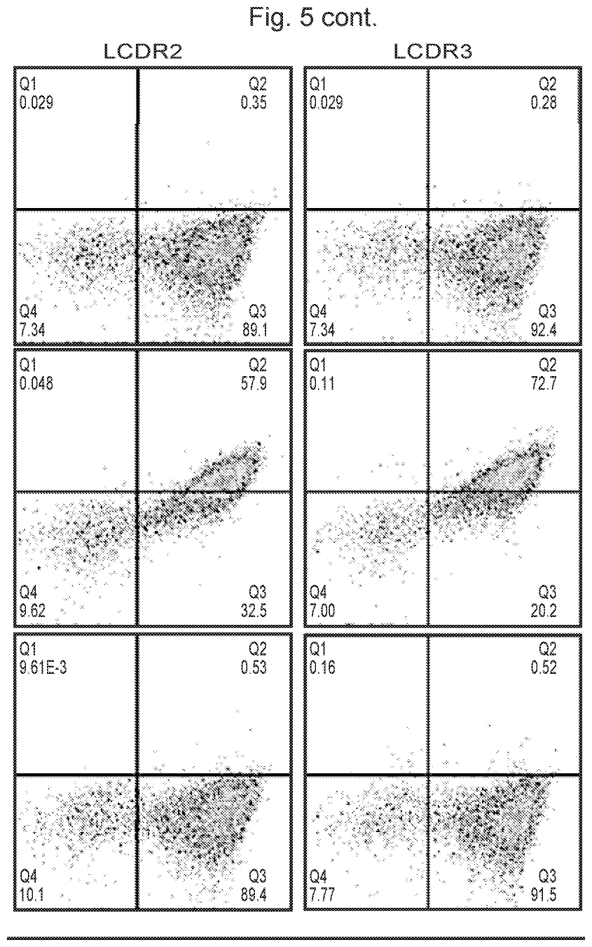

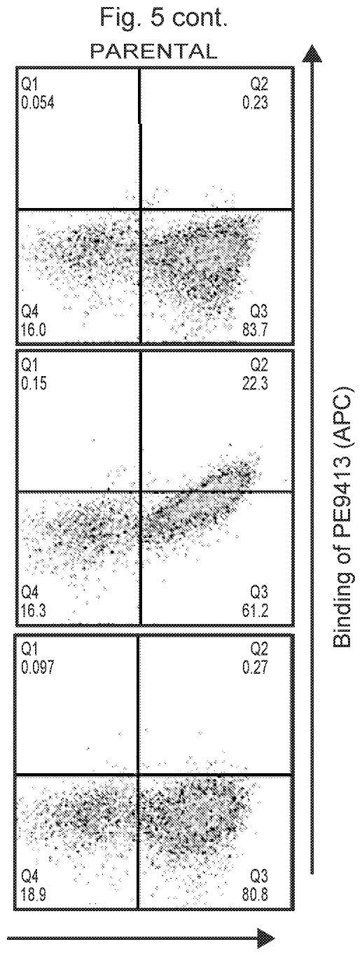

The 6 mutational scanning CDR libraries in yeast were also induced and stained with the target PE9413 using a lower concentration of the target peptide (85 nM) to increase the stringency of the selection. The end result of the second sort is shown in FIG. 5 where all libraries showed an apparent improved binding signal over the parental antibody when stained with the target peptide at 85 nM concentration.

Individual clones from each of the selected CDR library outputs were Sanger sequenced and mutational hotspots leading to binders specific to PE9413 were identified. The WebLogo representation [5] in FIG. 6 highlights sites of higher tolerance for mutations (e.g. LCDR2) and sites with a more restricted patter of accepted amino acid substitutions (e.g. HCDR3).

### Generation and Screening of a Combinatorial CDR Library

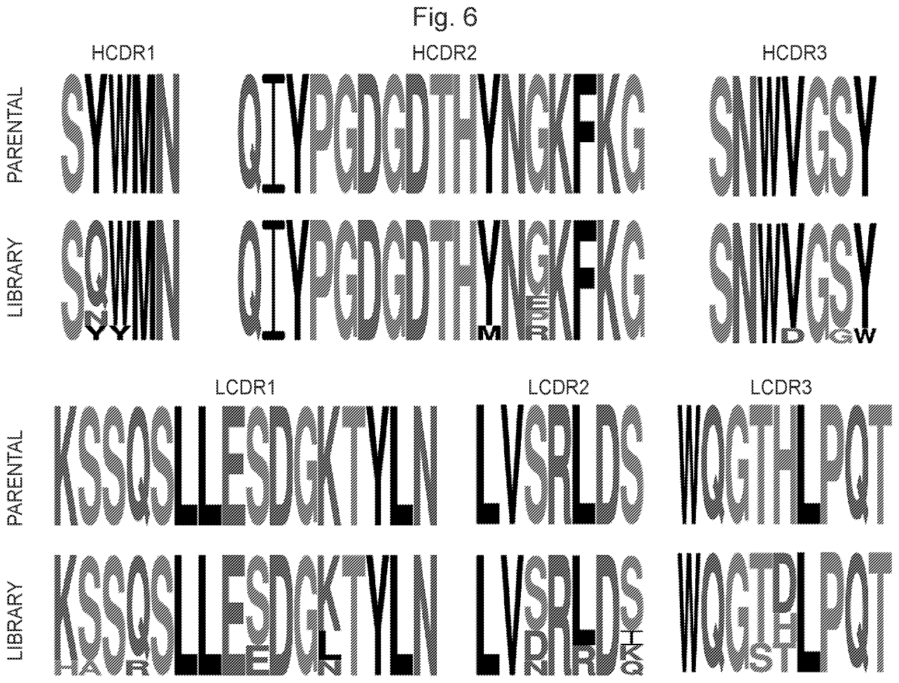

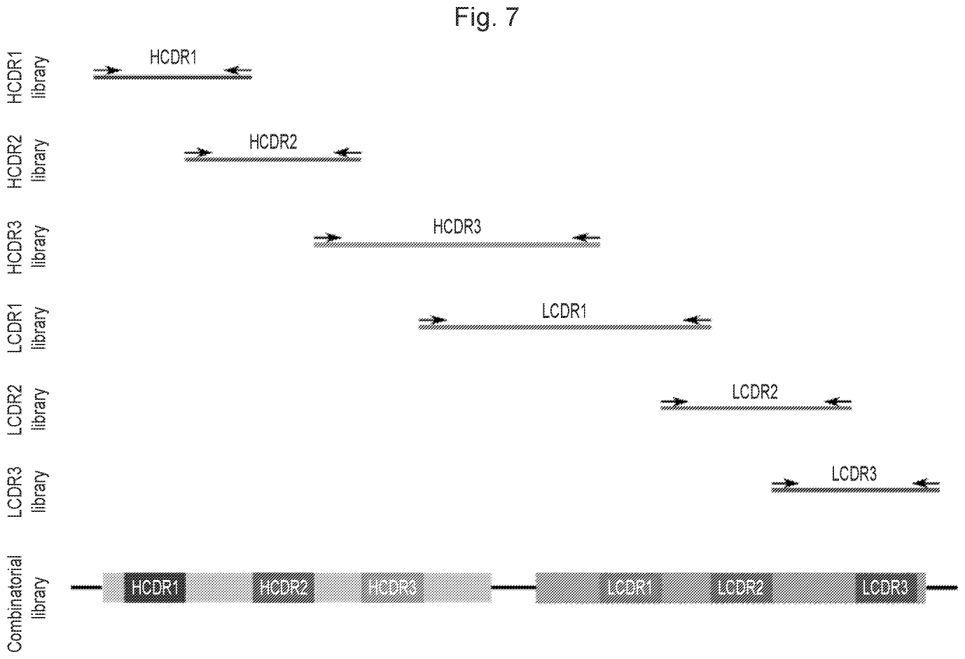

The CDRs selected after 2 rounds of sorting were PCR amplified from the six selected individual libraries and assembled in a final combinatorial library where each one of the parental CDRs was replaced by the collection of selected CDRs (FIG. 7). Yeast were transformed with the combinatorial library according to the protocols described above.

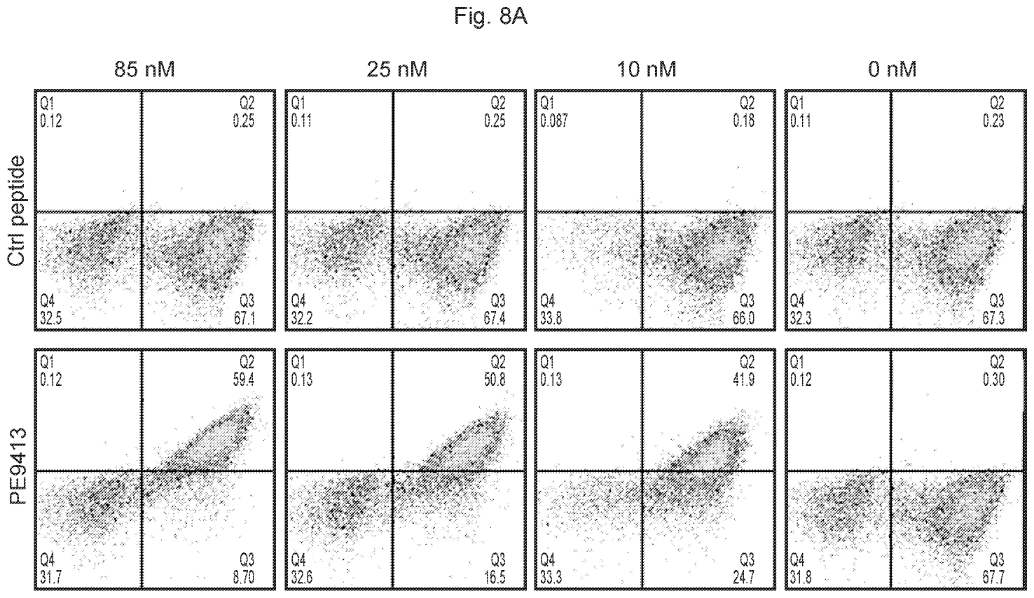

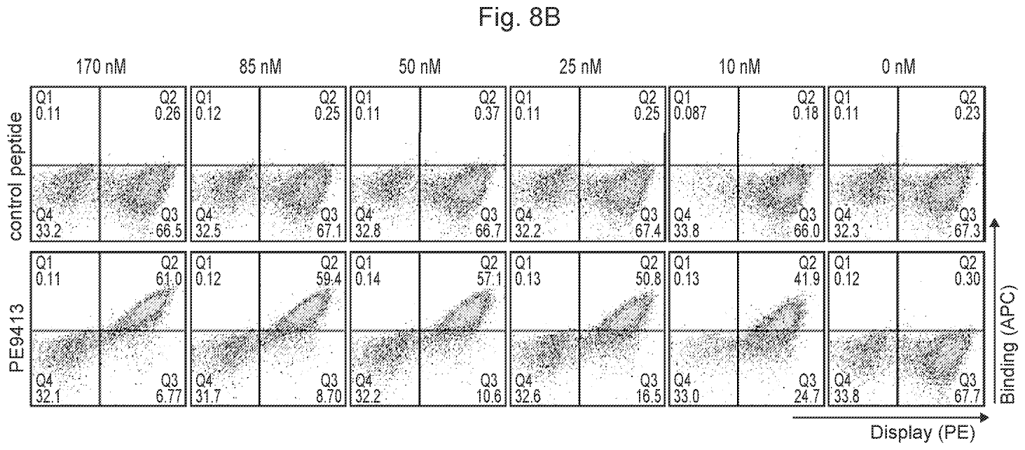

The final number of transformants in the combinatorial library was 1.33×10⁸. The library was induced and tested for binding to PE9413. Most clones in the library bound to PE9413 at a concentration as low as 10 nM with no detectable background (FIG. 8A). FIG. 8B shows binding of the clones to PE9413 at various concentrations (170 nM, 85 nM, 50 nM, 25 nM, 10 nM, 0 nM).

### Sorting of Combinatorial Library

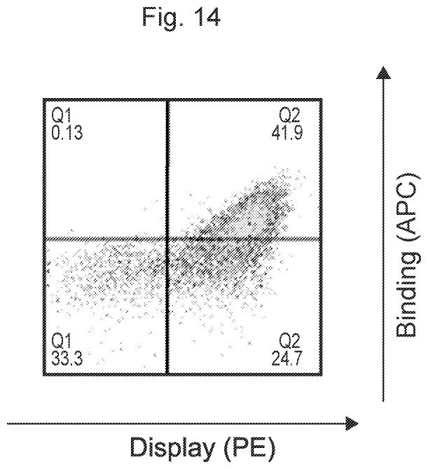

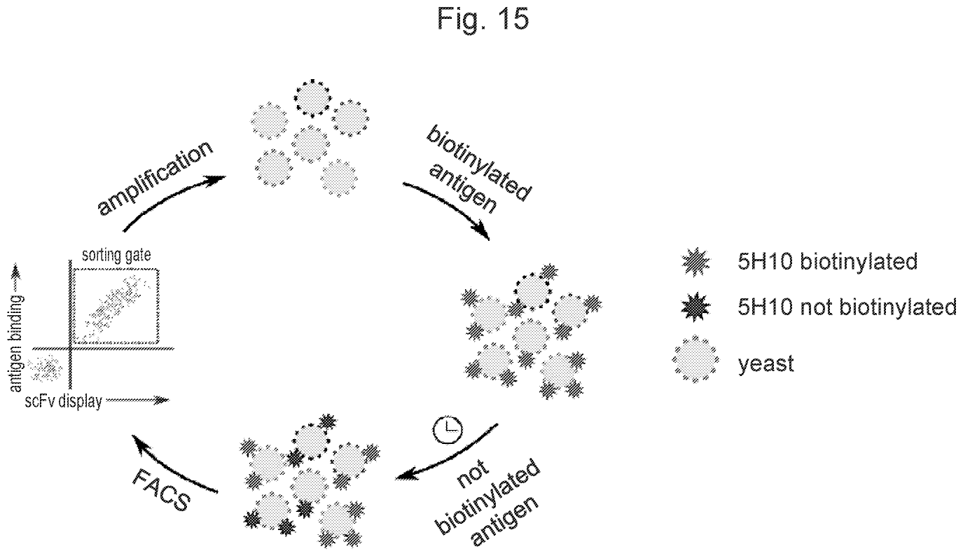

To reduce the size of the combinatorial library, the library was subjected to magnetic-activated cells sorting (MACS)[6] at 10 nM of PE9413. Briefly, the induced yeast cells were incubated with 10 nM of PE9413 in yeast wash buffer for 30 min in rotation at room temperature, then placed on ice for 5 min. After centrifugation, the cells were washed twice with yeast washing buffer and resuspended in yeast wash buffer. Streptavidin-coated paramagnetic beads were added to the solution and incubated on ice for 15 min with occasional mixing. Following 2 washing steps, the cells/beads mixture was resuspended in yeast wash buffer, and the sample was applied to a column set on a magnetic stand to allow for the retention of the yeast cells bound to PE9413 captured by streptavidin paramagnetic beads. After washing with yeast wash buffer, the column was removed from the magnetic stand and the yeast cells were eluted with yeast wash buffer. FIG. 14 shows a flow cytometry plot of the eluted population.

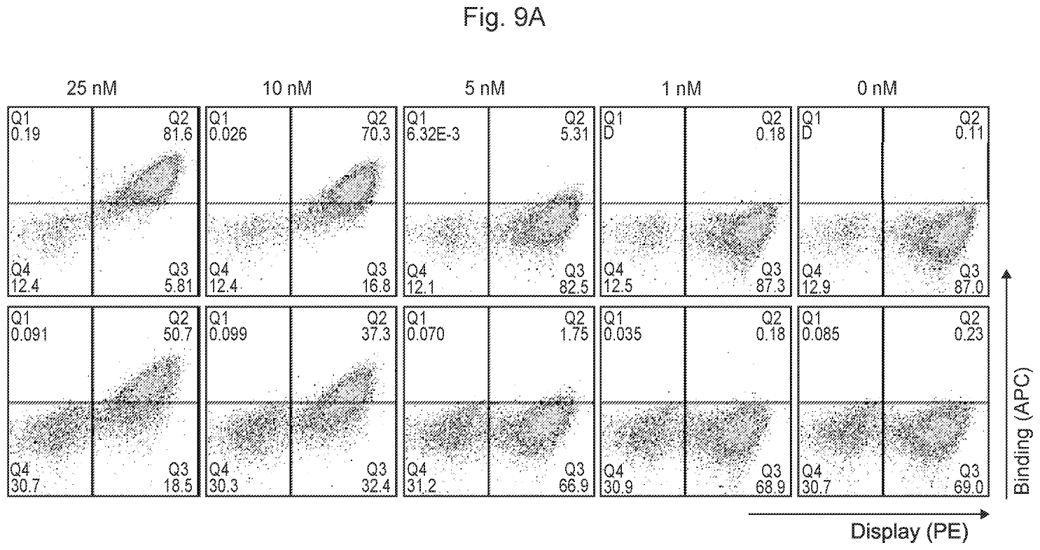

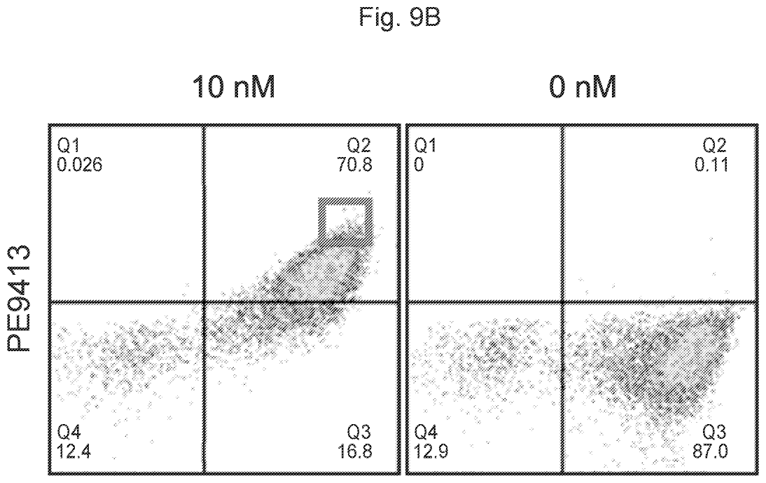

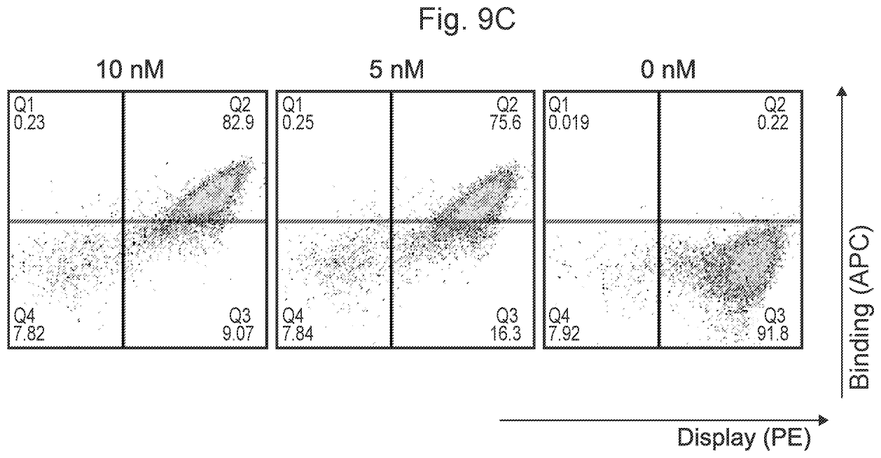

The MACS sorted population underwent a further equilibrium sort by fluorescence-activated cell sorting (FACS). The yeast cells displaying the combinatorial library were incubated with 25 nM, 10 nM, 5 nM, or 1 nM of PE9413 (FIG. 9A). The top 1% of yeast cells that bound to 10 nM of PE9413 were selected for (FIG. 9B). Binding of the sorted cells to 10 nM PE9413 is shown in FIG. 9C.

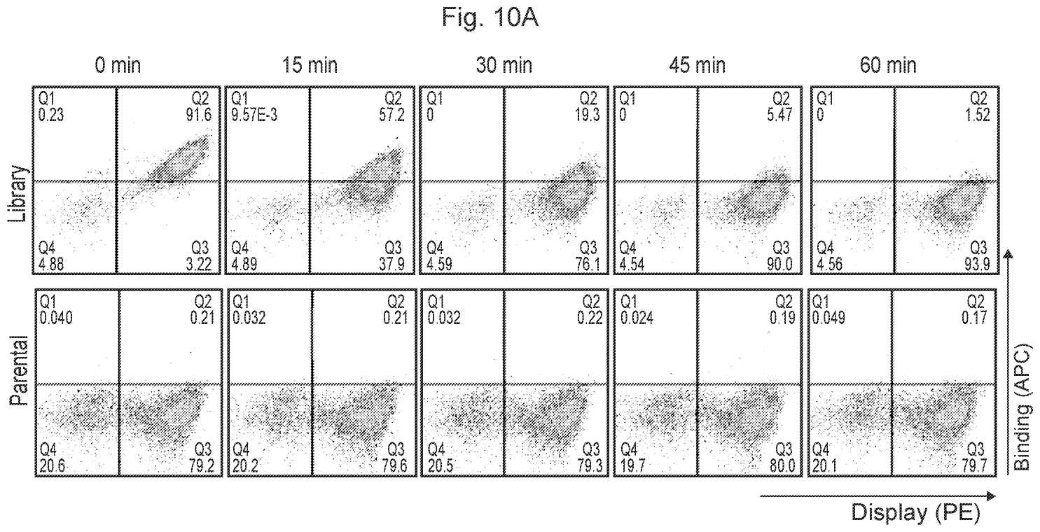

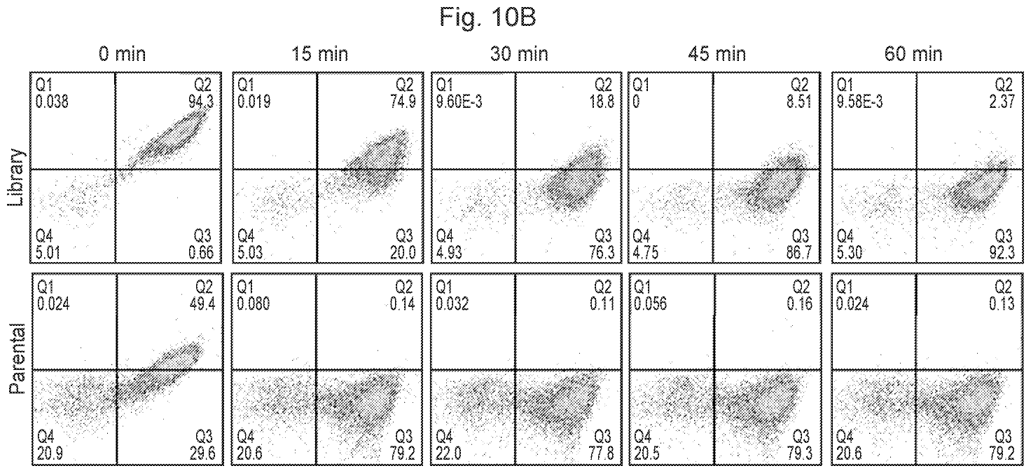

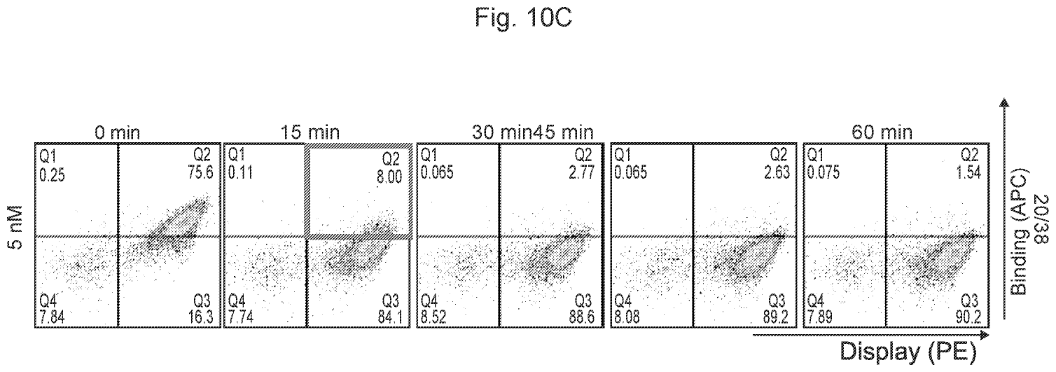

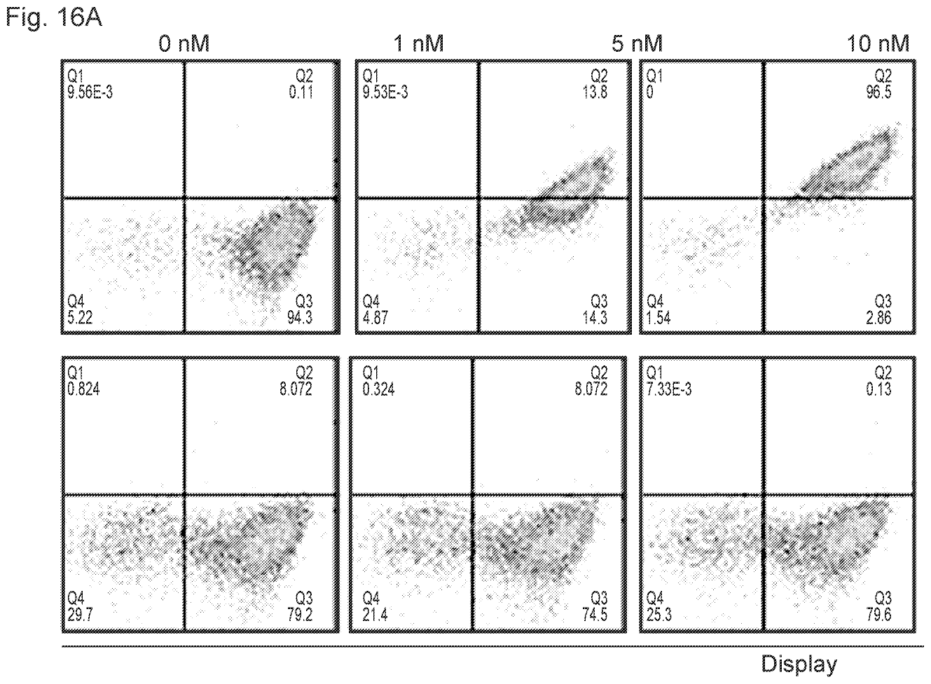

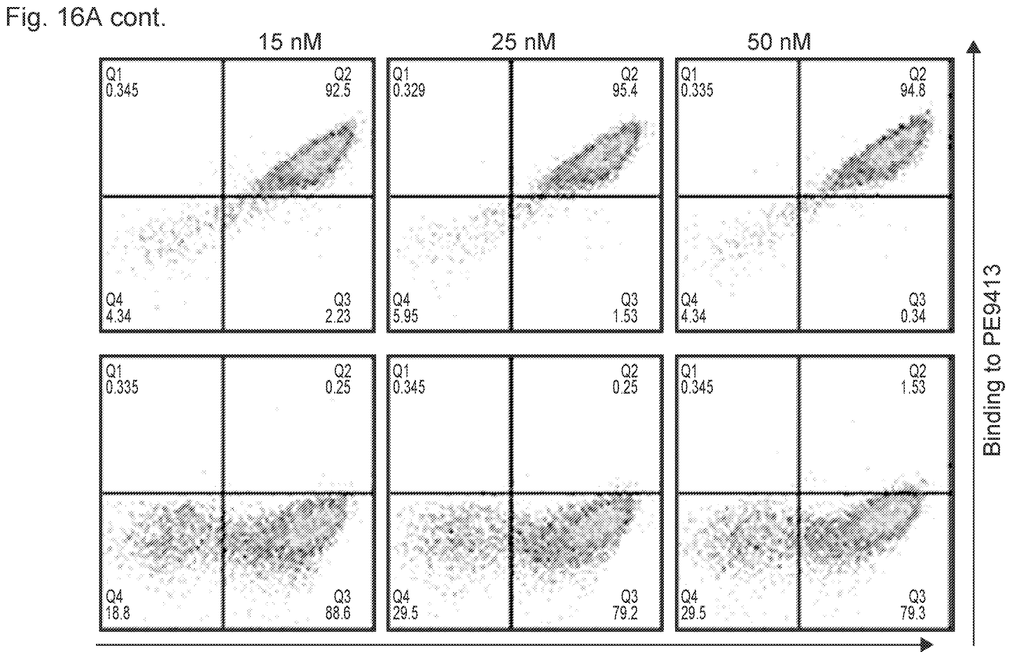

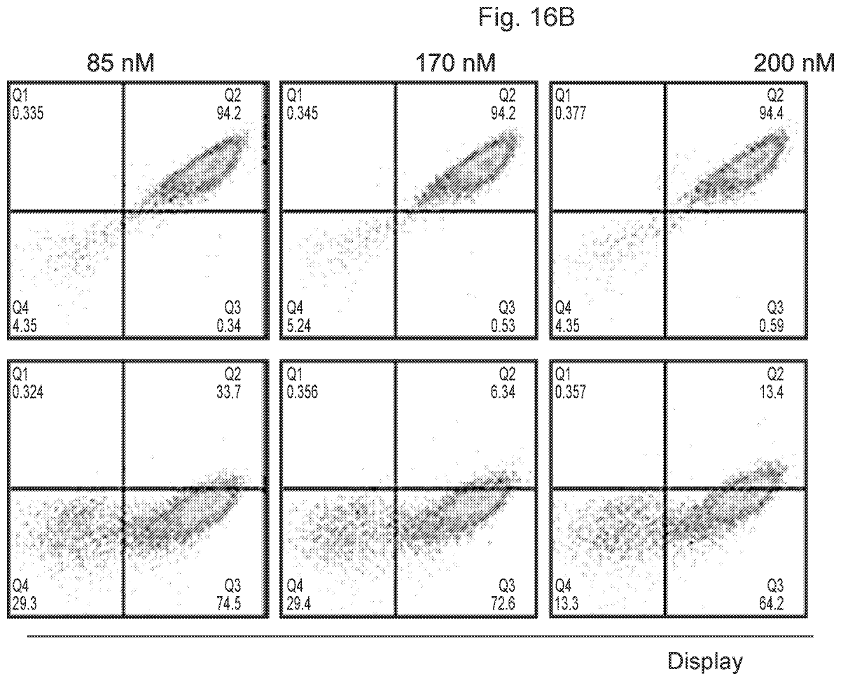

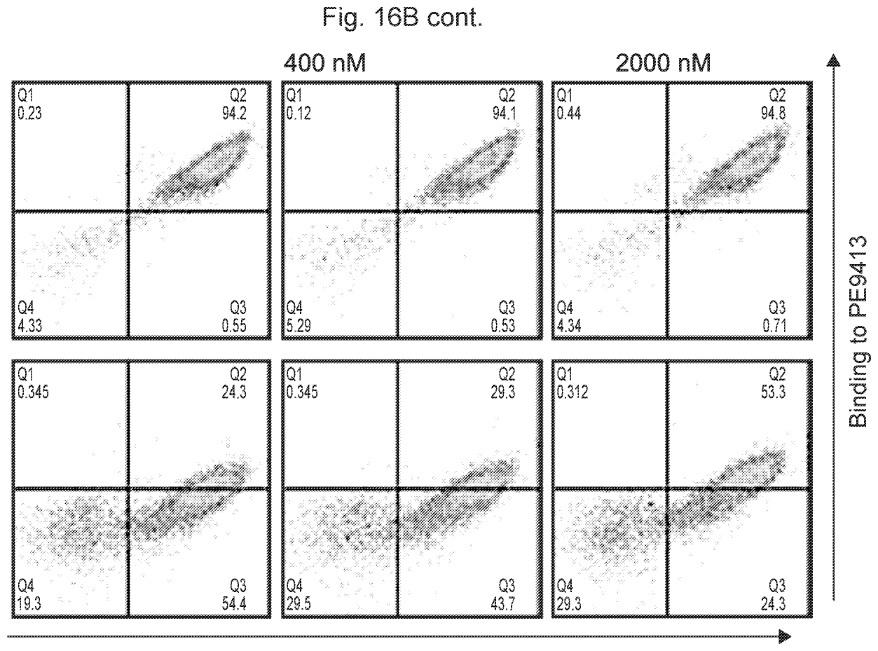

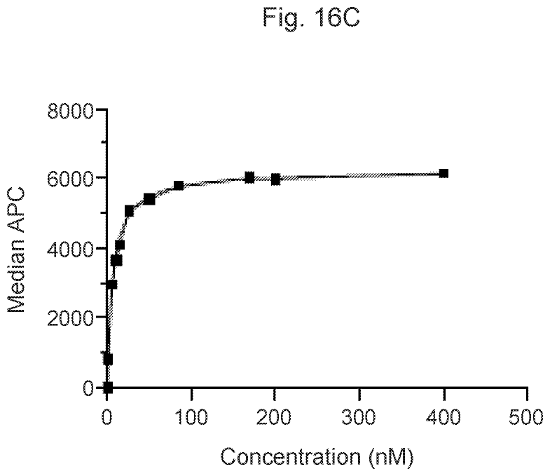

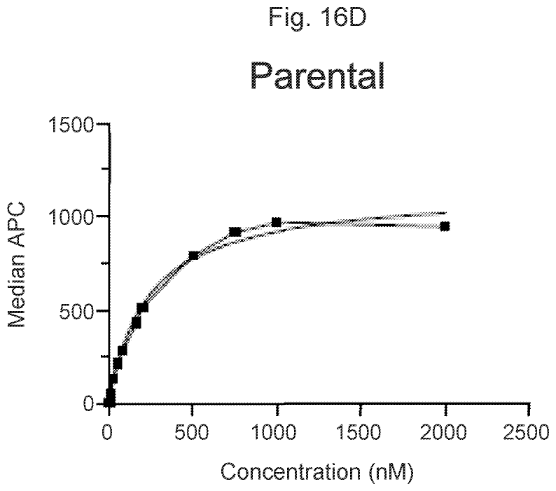

The FACS sorted population was sorted kinetically to find clones with an improved K_{off} compared to the parenteral scFv. The yeast cells were incubated with the biotinylated peptide PE9413, then washed and subsequently incubated with a 10-fold excess of unlabeled peptide to compete out the biotinylated antigen according to the off rate of the displayed scFv and prevent the re-binding of the biotinylated peptide. A time course study was conducted to identify the timepoint where the parental antibody would lose all the binding signal from the biotinylated peptide while the affinity matured polyclonal population would still show some binding. At 15 min of competition, the top 1% clones in the polyclonal population retaining binding were sorted. The result of the sort are shown in FIG. 10A, FIG. 10B, and FIG. 10C. Both the parental and sorted population were stained at equilibrium with the biotinylated peptide PE9413 and then incubated with the 10-fold excess of non-biotinylated target peptide for increasing amounts of time. The parental 5H10 shows complete loss of binding after 15 min, while the library retains detectable binding up to 1 hour of competition, indicating the selection of antibodies with improved K_{off.}. FIG. 16A and FIG. 16B show binding of the selected kinetically sorted yeast cells to 0 nM, 1 nM, 5 nm, 10 nM, 15 nM, 25 nM, 50 nM, 85 nM, 170 nM, 200 nM, 400 nM, and 2000 nM PE9413. FIG. 16C shows a plot of concentration of PE9413 (labeled “concentration”) versus the median fluorescent signal of the kinetically sorted yeast combinatorial library that bound to PE9413. The curve was used to determine that the affinity of the combinatorial library for C-terminal biotinylated PE9413 was 6.4 nM (R2=0.9964). FIG. 16D is a plot of concentration of PE9413 (labeled “concentration”) versus the median fluorescent signal of yeast cells displaying the parental 5H10 scFv. The curve was used to determine that the affinity of the parental 5H10 scFv for C-terminal biotinylated PE9413 was 232 nM (R2=0.9922).

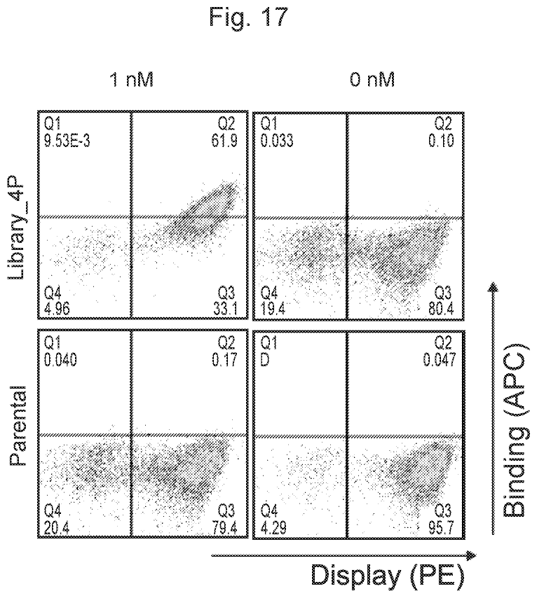

The kinetically sorted population underwent a final equilibrium sort by FACS using 1 nM of PE9413 to identify clones with an overall improved K_{D}. FIG. 17 shows flow cytometry plots of the final equilibrium sort.

### Sanger Sequencing of Affinity Matured Clones

95 clones from the kinetic sort and 95 clones from the final equilibrium sort were sequenced. A total of 137 unique sequences (SEQ ID NOS: 481-617) were identified of which 47 were selected based on frequency for further screenings (SEQ ID NOS: 481-527). FIG. 11 shows WebLogo representations for all the selected CDR sequences. Preferred mutations were observed at specific hotspots in HCDR1 (Y→Q/N), LCDR1 (K→N) and LCDR3 (H→D).

The binding of the selected 48 clones described above to 170 nM of C-terminal biotinylated PE9413 was evaluated. The 24 clones with the highest binding signal were further evaluated for binding affinity. Each clone was incubated with increasing amounts of biotinylated PE9413, and stained with a fluorescently labeled anti-streptavidin antibody. Binding of the clone to PE9413 was analyzed by flow cytometry. A plot of concentration of PE9413 versus the median fluorescent signal of the yeast cell population that bound to PE9413 was used to estimate the binding affinity (K_{D}) of each clone to C-terminal biotinylated PE9413. The K_{D} of 27 unique antibodies was measured and reported in Table A2, along with the R² value, indicative of the accuracy of the fitting of the experimental data.

TABLE A2
Binding Affinities of 27 Unique Clones
to C-terminal biotinylated PE9413

| Clone | K_{D} (nM) | R squared |
|---|---|---|
| A84P | 2.523 | 0.9937 |
| A64P | 3.294 | 0.9925 |
| B114P | 3.631 | 0.9945 |
| A104P | 3.636 | 0.9943 |
| B124P | 3.817 | 0.9946 |
| H74P | 3.843 | 0.9898 |
| H63P | 4.034 | 0.9906 |
| H83P | 4.078 | 0.9922 |
| F54P | 4.093 | 0.9941 |
| D44P | 4.185 | 0.9953 |
| D14P | 4.389 | 0.9925 |
| B104P | 4.518 | 0.9956 |
| B53P | 4.966 | 0.9917 |
| B34P | 5.056 | 0.9937 |
| B23P | 5.302 | 0.9905 |
| A44P | 5.32 | 0.9951 |
| F103P | 5.472 | 0.9938 |
| G24P | 5.795 | 0.9928 |
| G14P | 5.934 | 0.9944 |
| E94P | 6.334 | 0.9947 |
| D74P | 6.376 | 0.9947 |
| F64P | 6.686 | 0.9939 |
| D124P | 6.97 | 0.994 |
| G104P | 7.093 | 0.9952 |
| D113P | 8.073 | 0.9926 |
| E93P | 9.176 | 0.9966 |
| D24P | 14.41 | 0.996 |

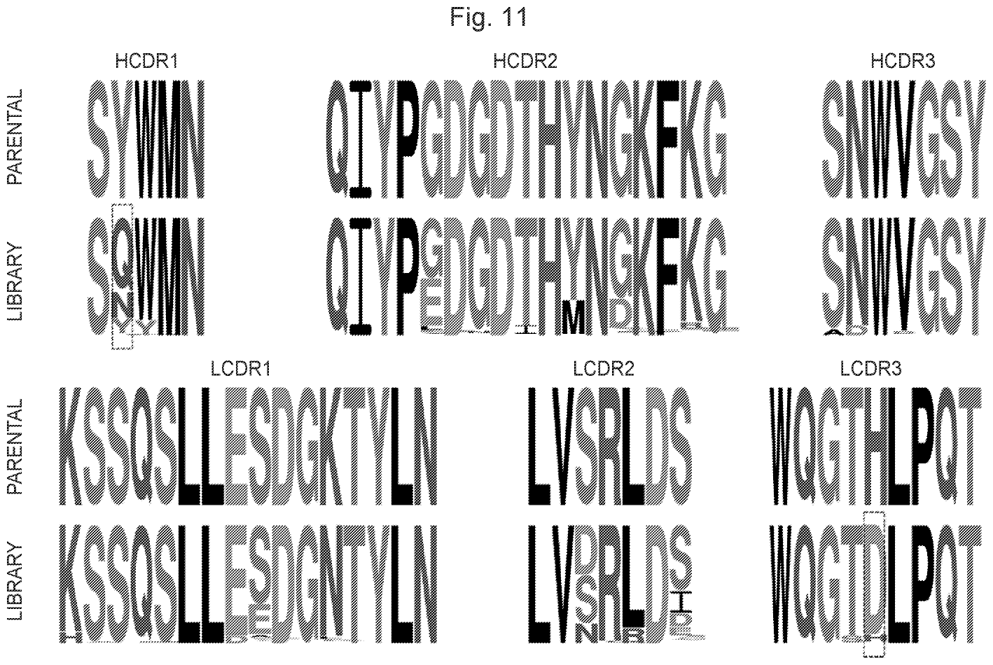

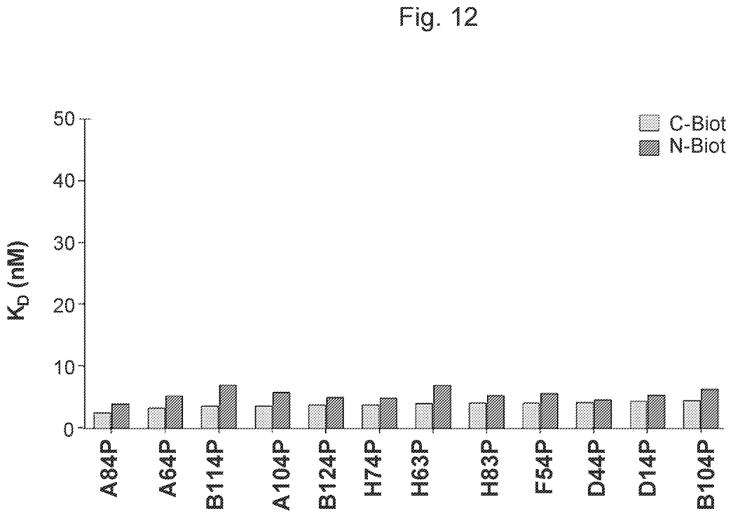

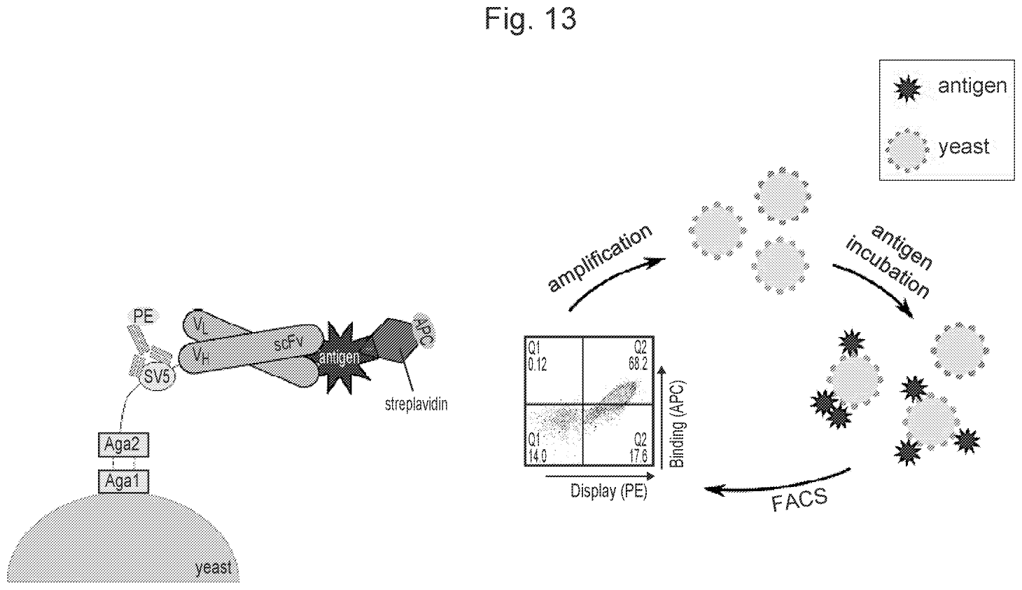

The affinity of the 12 best clones for the N-terminal biotinylated target peptide (PE9411) was also evaluated. Each clone was incubated with increasing amounts of N-terminal biotinylated PE9413, and stained with a fluorescently labeled anti-streptavidin antibody. Binding of the clone to N-terminal biotinylated PE9413 was analyzed by flow cytometry. A plot of concentration of PE9413 versus the median fluorescent signal of the yeast cell population that bound to PE9413 was used to estimate the binding affinity (K_{D}) of each clone to N-terminal biotinylated PE9413. The tested clones were confirmed to have improved affinities over the parental antibody 5H10 against the target sequence PE9413, regardless of the biotinylation modification, as reported in Table A3 and FIG. 12.

TABLE A3
Binding Affinities of Top 12 Clones
to N-terminal biotinylated PE9413

| Clone | K_{D} (nM) C-ter PE9413 | K_{D} (nM) N-ter PE9413 |
|---|---|---|
| A84P | 2.523 | 3.927 |
| A64P | 3.294 | 5.229 |
| B114P | 3.631 | 7.138 |
| A104P | 3.636 | 5.846 |
| B124P | 3.817 | 5.000 |
| H74P | 3.843 | 4.901 |
| H63P | 4.034 | 6.948 |
| H83P | 4.078 | 5.301 |
| F54P | 4.093 | 5.663 |
| D44P | 4.185 | 4.609 |
| D14P | 4.389 | 5.382 |
| B104P | 4.518 | 6.378 |

### Example 3: Evaluation of the Binding to Exon 6 Peptide by ELISA of Clones of Example 2

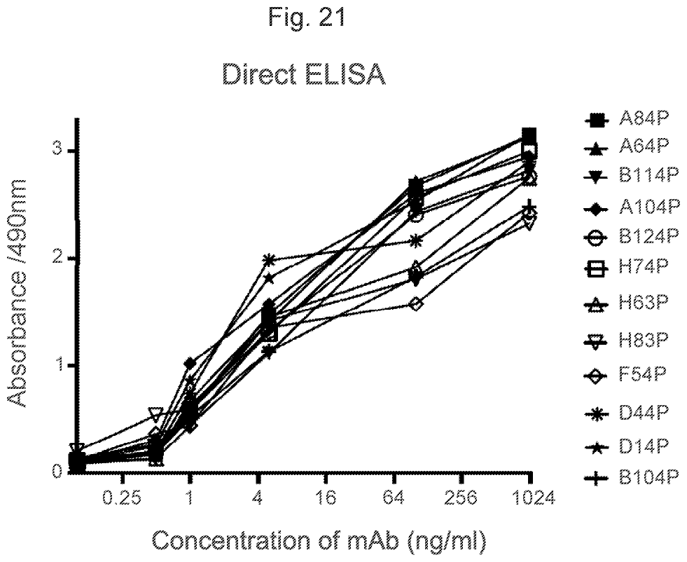

A direct enzyme-linked immunosorbent assay (ELISA) was performed to evaluate binding of the clones of Example 2 (referred to in all additional examples as “mutants”) to the Exon 6 peptide. The mutants were expressed as full length IgG4 antibodies. FIG. 21 shows the binding of mutant as a function of mutant concentration.

Table A4 below shows the absorbance at various mutant concentrations. A greater absorbance means more binding of the mutant to exon 6 peptide at a particular concentration.

TABLE A4
Absorbance at different variant concentrations

| | 1000 | 100 | 5 | 1 | 0.5 | 0.1 |
|---|---|---|---|---|---|---|
| | ng/mL | ng/mL | ng/mL | ng/mL | ng/mL | ng/mL |
| A84P | 3.1445 | 2.67 | 1.475 | 0.5605 | 0.193 | 0.131 |
| A64P | 3.1245 | 2.7126 | 1.4055 | 0.5635 | 0.1715 | 0.122 |
| B114P | 2.814 | 2.4335 | 1.115 | 0.446 | 0.127 | 0.098 |
| A104P | 2.943 | 2.599 | 1.57 | 1.0175 | 0.3055 | 0.092 |
| B124P | 2.76 | 2.4025 | 1.322 | 0.6295 | 0.183 | 0.0985 |
| H74P | 2.995 | 2.564 | 1.299 | 0.58 | 0.249 | 0.1055 |
| H63P | 2.746 | 1.916 | 1.449 | 0.6755 | 0.1365 | 0.0875 |
| H83P | 2.322 | 1.807 | 1.4165 | 0.5965 | 0.537 | 0.2045 |
| F54P | 2.42 | 1.575 | 1.349 | 0.4455 | 0.367 | 0.11 |
| D44P | 2.897 | 2.161 | 1.981 | 0.7435 | 0.207 | 0.0895 |
| D14P | 3.1515 | 2.5315 | 1.811 | 0.8605 | 0.2655 | 0.137 |
| B104P | 2.4815 | 1.827 | 1.131 | 0.527 | 0.174 | 0.0855 |

Methods: A 96-well plate was coated with 0.1-0.5 μg/mL of exon 6 peptide (ASSLRNDSSSSNRKAKNPPGDS, SEQ ID NO: 479) in coating buffer. The plate was incubated at 4° C. overnight. A control plate was coated with an alternative peptide that does not bind to the mutants. Both plates were washed with wash buffer twice. Then, the plates were blocked with blocking buffer for 1 hour at 37° C. The plates were then washed with wash buffer twice. The mutants were added to the plate at a concentration of 0.1 μg/well. The plate was covered and incubated for 1 hour at 37° C. The plates were then washed with wash buffer three times. The secondary antibody was added to the plates. The plates were covered and incubated for 1 hour at 37° C. The plates were then washed with wash buffer three times. 100 μL of detection substrate was added to each well of the plates. The plates were covered and incubated for 10-20 minutes at room temperature. Subsequently, 100 μL of stop solution was added to the plates. Each plate was read at an absorbance of 450 nm.

Reagents: Coating buffer contained 1.5M NaCl, 0.5M H₃BO₄, and 1.0N NaOH. Wash buffer contained 0.05% Tween-20 in PBS. Blocking buffer contained 2.0% normal goat serum in wash buffer. The dilution buffer was wash buffer and 2% FCS. The secondary antibody was a goat anti-human IgG HRP conjugate, purchased from Millipore (Catalogue Number: AP309P). The secondary antibody was diluted 1:1000. The detection substrate was prepared by dissolving 4 O-phenylenediamine dihydrochloride (OPD) tablets dissolved in 12 mL sterile distilled water. 5 μL of hydrogen peroxide (30% v/v) was added to the detection substrate immediately before adding substrate to the wells. The stop solution contained 0.5 M H₂SO₄.

### Example 4: Inhibition of c-Kit Activation by Mutants of Example 2

Purpose: The ability of the mutants to inhibit C-kit activation was evaluated. C-kit activation results in expression of inflammatory cytokines like CCL2 and TGFβ.

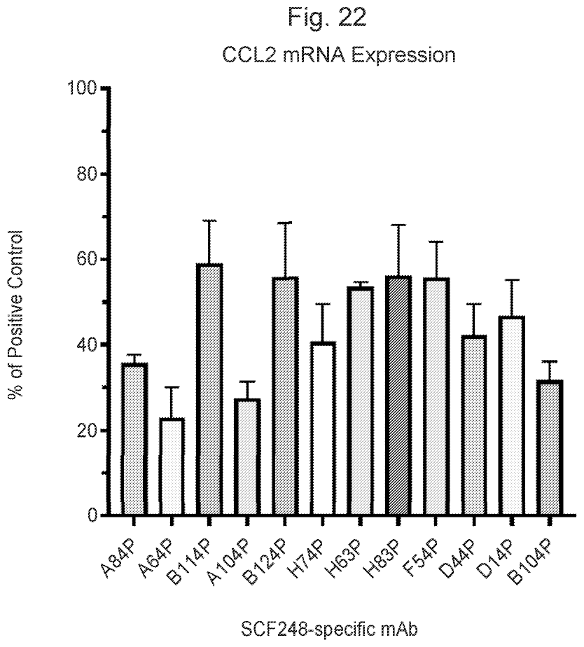

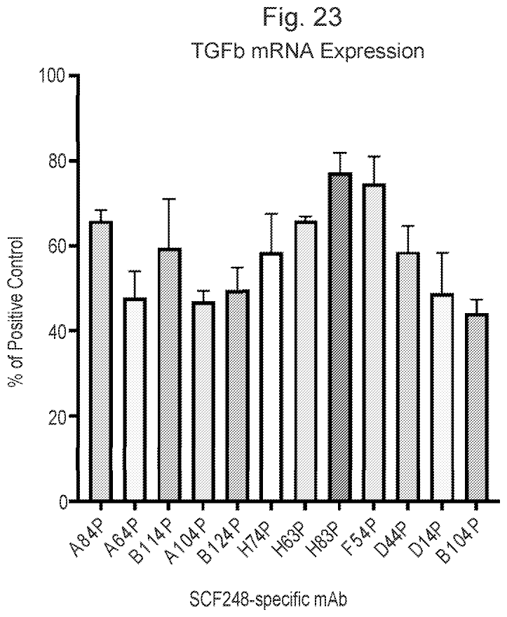

Results: FIG. 22 shows that in comparison to the positive control CCL2 mRNA expression was reduced. FIG. 23 shows that in comparison to the positive control TGFβ mRNA expression was reduced. Collectively, this data shows the ability of the mutants to reduce inflammation.

Table A5 shows the expression of CCL2 and TGFβ as a percentage of the positive control.

TABLE A5
Expression of CCL2 and TGFβ
as a Percentage of the Positive Control

| | CCL2 | | TGFβ | |
|---|---|---|---|---|
| | Expression as | | Expression as | |
| | Percentage of | Standard | Percentage of | Standard |
| | Positive Control | error | Positive Control | error |
| A84P | 35.6 | 2.1 | 65.8 | 2.6 |
| A64P | 22.9 | 7.4 | 47.9 | 6.2 |
| B114P | 59.1 | 10.1 | 59.5 | 11.7 |
| A104P | 27.4 | 4.0 | 46.9 | 2.6 |
| B124P | 55.9 | 12.9 | 49.6 | 5.3 |
| H74P | 40.6 | 9.0 | 58.4 | 9.0 |
| H63P | 53.5 | 1.4 | 65.6 | 1.2 |
| H83P | 56.2 | 12.1 | 77.1 | 4.8 |
| F54P | 55.7 | 10.1 | 74.6 | 6.5 |
| D44P | 42.2 | 7.4 | 58.5 | 6.1 |
| D14P | 46.8 | 8.6 | 48.8 | 9.8 |
| B104P | 31.7 | 4.5 | 43.9 | 3.6 |

Methods: A 24 well plate was coated with SI/SI4 hSCF cells (200,000 cells per well in 200 μL of medium) and incubated in the presence of 5% CO2 and 37° C. for 24 hours. LAD2 cells were suspended in serum free medium in the absence of SCF and seeded in a T75 tissue culture flask and incubated in the presence of 5% CO2 and 37° C. for 24 hours.

After 24 hours, LAD2 cells are centrifuged and resuspended in serum free medium with nutrients in the absence of SCF at a concentration of one million cells per milliliter. The media was aspirated from each well of the 24 well plate containing SI/SI4 hSCF cells. 250 μL of LAD2 cells were added to each well containing SI/SI4 hSCF cells. A mutant was added to the each well of the plate at concentration of 1 μg/mL and 10 μg/mL. The mutants were expressed as full length IgG4 antibodies. As a negative control, human IgG4 isotype control was added to the plate. The plate was incubated in the presence of 5% CO2 and 37° C. for 24 hours.

After 24 hours, the plate was centrifuged, and the supernatant was removed. RNA was extracted from the cells using a TRIZOL® reagent (an acid-guanidinumphenol based reagent). CCL2 and TGFβ mRNA was quantitated and compared to a positive control. The positive control is RNA extracted from the same cells that were not exposed to a mutant.

Reagents: A mouse SI/SI4 cell line (referred to herein as “SI/SI4 hSCF cells”) expressing human SCF248 was employed. The cell line was purchased from the American Type Culture Collection (ATCC CRL-2454). The SI/SI4 hSCF cells were grown on 0.1% gelatin (ATCC Cat PCT-999-027). LAD2 cells were also employed. LAD2 cells do not express human SCF248. LAD2 cells are SCF-responsive and depend on c-kit for activation. LAD2 cells are grown in the presence of 100 ng/mL recombinant human SCF in serum free media (STEMPRO™-34), which contains nutrient supplements.

### Example 5: Binding of Mutants of Example 2 to Cells Expressing SCF248 Via Flow Cytometry

The ability of the mutants to bind to cells expressing SCF248 is evaluated.

Cell Lines: A mouse SI/SI4 cell line expressing human SCF248 (referred to herein as “SI/SI4 hSCF248 cells”) and a mouse SI/SI4 expressing human SCF220 was employed. These cell lines were purchased from the American Type Culture Collection (ATCC CRL-2454; ATCC CRL-2453). The cells are grown on 0.1% gelatin (ATCC Cat PCT-999-027). Cells are positively selected over three passages using 100 μg/mL hygromycin B solution (Invitrogen 10687010) at the optimal dose as determined by cell viability.

Preparation of cells for flow cytometry staining. Cells are dissociated using a trypsin solution (0.25% trypsin in 2.2 mM ethylenediaminetetraacetic acid (EDTA)). The cells are washed twice by centrifugation at 300 g for four minutes. The cells are resuspended at a concentration of 1×10⁷ cells/mL for flow cytometry straining.

Flow cytometry staining. The cells are blocked in PBS containing blocking buffer (2% goat serum, 0.1% bovine serum albumin, and 0.1% TWEEN® 20 (polyoxyethylene sorbitol ester)) at 4° C. The cells are resuspended in blocking buffer containing a variant or the parental antibody of Example 1. As a negative control, cells are suspended in blocking buffer containing human IgG4 isotype control (BIOLEGEND® 403702). The cells are incubated for two hours at 4° C. The cells are washed twice and resuspended in blocking buffer containing the secondary antibody goat anti-human IgG-PE (INVITROGEN PAI-86078). The secondary antibody are diluted in the blocking buffer 1:500. The cells are protected from light and incubated for one hour at 4° C. The cells are washed twice and fixed in 2% paraformaldehyde (PFA) for ten minutes at 4° C.

Binding of the variants or parental antibody is evaluated via flow cytometry using an ACEA NOVOCYTE® flow cytometer. NovoExpress® software is utilized to control data collection and analysis on the flow cytometer. The Flow Jo 9.9.6 analysis program is utilized to analyze the data.

### Example 6: Internalization of Parental Antibody and Mutants of Example 2 Via Flow Cytometry

An assay is performed to evaluate internalization of the parental antibody and the mutants of Example 2.

The parental antibody, a mutant antibody, or a control antibody is labeled using Molecular Probes pHrodo Red Microscale Labeling Kit as directed by the manufacturer (ThermoFisher Scientific; Catalog #P35372). The pHrodo red is a pH sensitive dye that only fluoresces at pH lower than 6.5, which only occurs in the intracellular compartment of the endosome for this assay.

Once labeled, the antibody is incubated at room temperature with ATCC cell line SL/SL4 hSCF248 (CRL-2454) that expresses only the SCF248, and not SCF220, on its surface. Various time points are analyzed by live cell flow cytometry.

In order to slow the reaction at a particular time point the cells are put on ice and quickly analyzed by flow cytometry. The time points that are performed are 5, 15, 30 and 60 minutes. The cells have internalization (fluorescence) by 5 minutes and plateau by around 15 minutes of incubation, indicating a fast internalization of the pHRodo red tagged antibody once it interacts with the surface protein, SCF248.

Controls are run with the ATCC cell line SL/SL3 hSCF220 (CRL-2453) that only expresses SCF220 as well as ATCC cell line SL/SL2 control (CRL-2452). ATCC cell line SL/SL2 control cells do not express any SCF isoforms. Neither of these cell lines show internalization and demonstrate specificity for the parental antibody.

### REFERENCES

1. Boder, E. T. and K. D. Wittrup, Yeast surface display for screening combinatorial polypeptide libraries. Nat Biotechnol, 1997. 15(6): p. 553-7.
2. Ferrara, F., et al., Using phage and yeast display to select hundreds of monoclonal antibodies: application to antigen 85, a tuberculosis biomarker. PLoS One, 2012. 7(11): p. e49535.
3. Hunter, S. A. and J. R. Cochran, Cell-Binding Assays for Determining the Affinity of Protein-Protein Interactions: Technologies and Considerations. Methods Enzymol, 2016. 580: p. 21-44.
4. Orr-Weaver, T. L., J. W. Szostak, and R. J. Rothstein, Yeast transformation: a model systemfor the study ofrecombination. Proc Natl Acad Sci USA, 1981. 78(10): p. 6354-8.
5. Crooks, G. E., et al., WebLogo: a sequence logo generator. Genome Res, 2004. 14(6): p. 1188-90.
6. Abts, H., et al., CD20 positive human B lymphocytes separated with the magnetic cell sorter (MACS) can be induced to proliferation and antibody secretion in vitro. J Immunol Methods, 1989. 125(1-2): p. 19-28.
7. Tiller, K. E., et al., Arginine mutations in antibody complementarity-determining regions display context-dependent affnity/specficity trade-offs. J Biol Chem, 2017. 292(40): p. 16638-16652.
8. Lowenthal, M. S., et al., Identfication of Novel N-Glycosylation Sites at Noncanonical Protein Consensus Motifs. J Proteome Res, 2016. 15(7): p. 2087-101.
9. Sydow, J. F., et al., Structure-based prediction of asparagine and aspartate degradation sites in antibody variable regions. PLoS One, 2014. 9(6): p. e100736.
10. Li, X., C. Lin, and P. B. O'Connor, Glutamine deamidation: differentiation of glutamic acid and gamma-glutamic acid in peptides by electron capture dissociation. Anal Chem, 2010. 82(9): p. 3606-15.
11. Kelly, R. L., et al., Reduction of Nonspecificity Motifs in Synthetic Antibody Libraries. J Mol Biol, 2018. 430(1): p. 119-130.
12. Alam, M. E., et al., Biophysical and Sequence-Based Methods for Identifying Monovalent and Bivalent Antibodies with High Colloidal Stability. Mol Pharm, 2018. 15(1): p. 150-163.
13. Vlasak, J. and R. Ionescu, Fragmentation of monoclonal antibodies. MAbs, 2011. 3(3): p. 253-63.

### Numbered Embodiments of the Disclosure

Notwithstanding the appended claims, the disclosure sets forth the following numbered embodiments:
1. An antibody or fragment thereof that specifically binds to stem cell factor (SCF), wherein the antibody comprises a heavy chain and a light chain, the heavy and light chain each comprising three complementarity determining regions (CDRs), comprising:
   (i) a heavy chain CDR1 comprising an amino acid sequence according to SEQ ID NO: 1 (SX₂X₃MN, wherein X₂ is Q, N, or Y; and X₃ is W or Y);
   (ii) a heavy chain CDR2 comprising an amino acid sequence according to SEQ ID NO: 2 (QIYPX₅DX₇DX₉HX₁₁NX₁₃KFX₁₆X₁₇, wherein X₅ is E, G, D, or L; X₇ is G, D or N; X₉ is T or I; X₁₁ is M or Y; X₁₃ is G, D, or E; X₁ is K, R, N, E, or D; and X₁₇ is G or T);
   (iii) a heavy chain CDR3 comprising an amino acid sequence according to SEQ ID NO: 3 (X₁NWX₄GSY, wherein X₁ is S or A; and X₄ is V or D);
   (iv) a light chain CDR1 comprising an amino acid sequence according to SEQ ID NO: 4 (X₁X₂SQSLLX₈X₉DGNTYLN, wherein X₁ is K or H; X₂ is S or A; X₈ is E or D; and X₉ is S, E, Q, A, or G);
   (v) a light chain CDR2 comprising an amino acid sequence according to SEQ ID NO: 5 (LVX₃RX₅DX₇, wherein X₃ is D, N, or S; X₅ is L or R; and X₇ is I, D, S, or L); and
   (vi) a light chain CDR3 comprising an amino acid sequence according to SEQ ID NO: 6 (WQGX₄X₅LPQT, wherein X₄ is T or S; and X₅ is D or H).
2. The antibody or fragment thereof of embodiment 1, comprising:
   (i) a heavy chain CDR1 comprising an amino acid sequence according to SEQ ID NO: 7 (SX₂WMN, wherein X₂ is Q or N);
   (ii) a heavy chain CDR2 comprising an amino acid sequence according to SEQ ID NO: 8 (QIYPX₅DX₇DX₉HX₁₁NX₁₃KFKX₁₇, wherein X₅ is E, G, or D; X₇ is G or D; X₉ is T or I; X₁₃ is G or D; X₁₁ is M or Y; and X₁₇ is G or T);
   (iii) a heavy chain CDR3 comprising an amino acid sequence according to SEQ ID NO: 9 (SNWX₄GSY, wherein X₄ is V or D);
   (iv) a light chain CDR1 comprising an amino acid sequence according to SEQ ID NO: 10 (KSSQSLLEX₉DGNTYLN, wherein X₉ is S, E, Q, or A);
   (v) a light chain CDR2 comprising an amino acid sequence according to SEQ ID NO: 11 (LVX₃RLDX₇, wherein X₃ is D or N; X₇ is I, D, S, or L); and
   (vi) a light chain CDR3 according to SEQ ID NO: 6 (WQGX₄X₅LPQT, wherein X₄ is T or S; and X₅ is D or H).
3. The antibody or fragment thereof of embodiment 1, comprising:
   (i) a heavy chain CDR1, CDR2, and CDR3 according to SEQ ID Nos: 22, 26, and 16, respectively; and a light chain CDR1, CDR2, and CDR3 according to SEQ ID NOs: 71, 90, and 111;
   (ii) a heavy chain CDR1, CDR2, and CDR3 according to SEQ ID Nos: 23, 27, and 16, respectively; and a light chain CDR1, CDR2, and CDR3 according to SEQ ID NOs: 71, 91, and 111;
   (iii) a heavy chain CDR1, CDR2, and CDR3 according to SEQ ID Nos: 22, 26, and 67, respectively; and a light chain CDR1, CDR2, and CDR3 according to SEQ ID NOs: 71, 92, and 111;
   (iv) a heavy chain CDR1, CDR2, and CDR3 according to SEQ ID Nos: 22, 27, and 16, respectively; and a light chain CDR1, CDR2, and CDR3 according to SEQ ID NOs: 71, 92, and 111;
   (v) a heavy chain CDR1, CDR2, and CDR3 according to SEQ ID Nos: 23, 28, and 16, respectively; and a light chain CDR1, CDR2, and CDR3 according to SEQ ID NOs: 72, 92, and 111;
   (vi) a heavy chain CDR1, CDR2, and CDR3 according to SEQ ID Nos: 22, 27, and 16, respectively; and a light chain CDR1, CDR2, and CDR3 according to SEQ ID NOs: 71, 93, and 112;
   (vii) a heavy chain CDR1, CDR2, and CDR3 according to SEQ ID Nos: 22, 28, and 16, respectively; and a light chain CDR1, CDR2, and CDR3 according to SEQ ID NOs: 73, 92, and 111;
   (viii) a heavy chain CDR1, CDR2, and CDR3 according to SEQ ID Nos: 22, 29, and 16, respectively; and a light chain CDR1, CDR2, and CDR3 according to SEQ ID NOs: 73, 92, and 111;
   (ix) a heavy chain CDR1, CDR2, and CDR3 according to SEQ ID Nos: 22, 30, and 16, respectively; and a light chain CDR1, CDR2, and CDR3 according to SEQ ID NOs: 71, 92, and 111;
   (x) a heavy chain CDR1, CDR2, and CDR3 according to SEQ ID Nos: 22, 31, and 16, respectively; and a light chain CDR1, CDR2, and CDR3 according to SEQ ID NOs: 71, 92, and 112;
   (xi) a heavy chain CDR1, CDR2, and CDR3 according to SEQ ID Nos: 22, 32, and 16, respectively; and a light chain CDR1, CDR2, and CDR3 according to SEQ ID NOs: 73, 93, and 111;
   (xii) a heavy chain CDR1, CDR2, and CDR3 according to SEQ ID Nos: 22, 33, and 16, respectively; and a light chain CDR1, CDR2, and CDR3 according to SEQ ID NOs: 74, 92, and 111;
   (xiii) a heavy chain CDR1, CDR2, and CDR3 according to SEQ ID Nos: 24, 34, and 16, respectively; and a light chain CDR1, CDR2, and CDR3 according to SEQ ID NOs: 73, 94, and 111;
   (xiv) a heavy chain CDR1, CDR2, and CDR3 according to SEQ ID Nos: 23, 28, an d16, respectively; and a light chain CDR1, CDR2, and CDR3 according to SEQ ID NOs: 73, 94, and 111;
   (xv) a heavy chain CDR1, CDR2, and CDR3 according to SEQ ID Nos: 22, 31, and 16, respectively; and a light chain CDR1, CDR2, and CDR3 according to SEQ ID NOs: 73, 92, and 111;
   (xvi) a heavy chain CDR1, CDR2, and CDR3 according to SEQ ID Nos: 22, 28, and 16, respectively; and a light chain CDR1, CDR2, and CDR3 according to SEQ ID NOs: 73, 92, and 111;
   (xvii) a heavy chain CDR1, CDR2, and CDR3 according to SEQ ID Nos: 22, 27, and 16, respectively; and a light chain CDR1, CDR2, and CDR3 according to SEQ ID NOs: 73, 92, and 111;
   (xviii) a heavy chain CDR1, CDR2, and CDR3 according to SEQ ID Nos: 22, 35, and 16, respectively; and a light chain CDR1, CDR2, and CDR3 according to SEQ ID NOs: 74, 92, and 111;
   (xix) a heavy chain CDR1, CDR2, and CDR3 according to SEQ ID Nos: 22, 34, and 16, respectively; and a light chain CDR1, CDR2, and CDR3 according to SEQ ID NOs: 71, 92, and 111;
   (xx) a heavy chain CDR1, CDR2, and CDR3 according to SEQ ID Nos: 22, 27, and 16, respectively; and a light chain CDR1, CDR2, and CDR3 according to SEQ ID NOs: 75, 92, and 111;
   (xxi) a heavy chain CDR1, CDR2, and CDR3 according to SEQ ID Nos: 24, 34, and 16, respectively; and a light chain CDR1, CDR2, and CDR3 according to SEQ ID NOs: 73, 92, and 111;
   (xxii) a heavy chain CDR1, CDR2, and CDR3 according to SEQ ID Nos: 22, 36, and 16, respectively; and a light chain CDR1, CDR2, and CDR3 according to SEQ ID NOs: 71, 95, and 111;
   (xxiii) a heavy chain CDR1, CDR2, and CDR3 according to SEQ ID Nos: 23, 28, and 16, respectively; and a light chain CDR1, CDR2, and CDR3 according to SEQ ID NOs: 73, 92, and 111;
   (xxiv) a heavy chain CDR1, CDR2, and CDR3 according to SEQ ID Nos: 22, 28, and 16, respectively; and a light chain CDR1, CDR2, and CDR3 according to SEQ ID NOs: 73, 96, and 111;
   (xxv) a heavy chain CDR1, CDR2, and CDR3 according to SEQ ID Nos: 22, 34, and 16, respectively; and a light chain CDR1, CDR2, and CDR3 according to SEQ ID NOs: 71, 95, and 111;
   (xxvi) a heavy chain CDR1, CDR2, and CDR3 according to SEQ ID Nos: 22, 34, and 16, respectively; and a light chain CDR1, CDR2, and CDR3 according to SEQ ID NOs: 79, 90, and 111;
   (xxvii) a heavy chain CDR1, CDR2, and CDR3 according to SEQ ID Nos: 22, 34, and 16, respectively; and a light chain CDR1, CDR2, and CDR3 according to SEQ ID NOs: 75, 92, and 111; or
   (xxviii) a heavy chain CDR1, CDR2, and CDR3 according to SEQ ID Nos: 22, 27, and 16, respectively; and a light chain CDR1, CDR2, and CDR3 according to SEQ ID NOs: 73, 98, and 111.
4. The antibody or fragment thereof of any one of embodiments 1-3, wherein the antibody comprises:
   (i) a heavy chain variable region comprising an amino acid sequence having at least 80% identity to SEQ ID NO: 114 and a light chain variable region comprising an amino acid sequence having at least 80% identity to SEQ ID NO: 291;
   (ii) a heavy chain variable region comprising an amino acid sequence having at least 80% identity to SEQ ID NO: 115 and a light chain variable region comprising an amino acid sequence having at least 80% identity to SEQ ID NO: 292;
   (iii) a heavy chain variable region comprising an amino acid sequence having at least 80% identity to SEQ ID NO: 116 and a light chain variable region comprising an amino acid sequence having at least 80% identity to SEQ ID NO: 293;
   (iv) a heavy chain variable region comprising an amino acid sequence having at least 80% identity to SEQ ID NO: 117 and a light chain variable region comprising an amino acid sequence having at least 80% identity to SEQ ID NO: 294;
   (v) a heavy chain variable region comprising an amino acid sequence having at least 80% identity to SEQ ID NO: 118 and a light chain variable region comprising an amino acid sequence having at least 80% identity to SEQ ID NO: 295;
   (vi) a heavy chain variable region comprising an amino acid sequence having at least 80% identity to SEQ ID NO: 119 and a light chain variable region comprising an amino acid sequence having at least 80% identity to SEQ ID NO: 296;
   (vii) a heavy chain variable region comprising an amino acid sequence having at least 80% identity to SEQ ID NO: 120 and a light chain variable region comprising an amino acid sequence having at least 80% identity to SEQ ID NO: 297;
   (viii) a heavy chain variable region comprising an amino acid sequence having at least 80% identity to SEQ ID NO: 121 and a light chain variable region comprising an amino acid sequence having at least 80% identity to SEQ ID NO: 298;
   (ix) a heavy chain variable region comprising an amino acid sequence having at least 80% identity to SEQ ID NO: 122 and a light chain variable region comprising an amino acid sequence having at least 80% identity to SEQ ID NO: 299;
   (x) a heavy chain variable region comprising an amino acid sequence having at least 80% identity to SEQ ID NO: 123 and a light chain variable region comprising an amino acid sequence having at least 80% identity to SEQ ID NO: 300;
   (xi) a heavy chain variable region comprising an amino acid sequence having at least 80% identity to SEQ ID NO: 124 and a light chain variable region comprising an amino acid sequence having at least 80% identity to SEQ ID NO: 301;
   (xii) a heavy chain variable region comprising an amino acid sequence having at least 80% identity to SEQ ID NO: 125 and a light chain variable region comprising an amino acid sequence having at least 80% identity to SEQ ID NO: 302;
   (xiii) a heavy chain variable region comprising an amino acid sequence having at least 80% identity to SEQ ID NO: 126 and a light chain variable region comprising an amino acid sequence having at least 80% identity to SEQ ID NO: 303;
   (xiv) a heavy chain variable region comprising an amino acid sequence having at least 80% identity to SEQ ID NO: 127 and a light chain variable region comprising an amino acid sequence having at least 80% identity to SEQ ID NO: 304;
   (xv) a heavy chain variable region comprising an amino acid sequence having at least 80% identity to SEQ ID NO: 128 and a light chain variable region comprising an amino acid sequence having at least 80% identity to SEQ ID NO: 305;
   (xvi) a heavy chain variable region comprising an amino acid sequence having at least 80% identity to SEQ ID NO: 129 and a light chain variable region comprising an amino acid sequence having at least 80% identity to SEQ ID NO: 306;
   (xvii) a heavy chain variable region comprising an amino acid sequence having at least 80% identity to SEQ ID NO: 130 and a light chain variable region comprising an amino acid sequence having at least 80% identity to SEQ ID NO: 307;
   (xviii) a heavy chain variable region comprising an amino acid sequence having at least 80% identity to SEQ ID NO: 131 and a light chain variable region comprising an amino acid sequence having at least 80% identity to SEQ ID NO: 308;
   (xix) a heavy chain variable region comprising an amino acid sequence having at least 80% identity to SEQ ID NO: 132 and a light chain variable region comprising an amino acid sequence having at least 80% identity to SEQ ID NO: 309;
   (xx) a heavy chain variable region comprising an amino acid sequence having at least 80% identity to SEQ ID NO: 133 and a light chain variable region comprising an amino acid sequence having at least 80% identity to SEQ ID NO: 310;
   (xxi) a heavy chain variable region comprising an amino acid sequence having at least 80% identity to SEQ ID NO: 134 and a light chain variable region comprising an amino acid sequence having at least 80% identity to SEQ ID NO: 311;
   (xxii) a heavy chain variable region comprising an amino acid sequence having at least 80% identity to SEQ ID NO: 135 and a light chain variable region comprising an amino acid sequence having at least 80% identity to SEQ ID NO: 312;
   (xxiii) a heavy chain variable region comprising an amino acid sequence having at least 80% identity to SEQ ID NO: 136 and a light chain variable region comprising an amino acid sequence having at least 80% identity to SEQ ID NO: 313;
   (xxiv) a heavy chain variable region comprising an amino acid sequence having at least 80% identity to SEQ ID NO: 137 and a light chain variable region comprising an amino acid sequence having at least 80% identity to SEQ ID NO: 314;
   (xxv) a heavy chain variable region comprising an amino acid sequence having at least 80% identity to SEQ ID NO: 149 and a light chain variable region comprising an amino acid sequence having at least 80% identity to SEQ ID NO: 326;
   (xxvi) a heavy chain variable region comprising an amino acid sequence having at least 80% identity to SEQ ID NO: 156 and a light chain variable region comprising an amino acid sequence having at least 80% identity to SEQ ID NO: 333;
   (xxvii) a heavy chain variable region comprising an amino acid sequence having at least 80% identity to SEQ ID NO: 158 and a light chain variable region comprising an amino acid sequence having at least 80% identity to SEQ ID NO: 335; or
   (xxviii) a heavy chain variable region comprising an amino acid sequence having at least 80% identity to SEQ ID NO: 143 and a light chain variable region comprising an amino acid sequence having at least 80% identity to SEQ ID NO: 320.
5. The antibody or fragment thereof of any one of embodiments 1-4, wherein the antibody comprises:
   (i) a heavy chain variable region comprising an amino acid sequence having at least 90% identity to SEQ ID NO: 114 and a light chain variable region comprising an amino acid sequence having at least 90% identity to SEQ ID NO: 291;
   (ii) a heavy chain variable region comprising an amino acid sequence having at least 90% identity to SEQ ID NO: 115 and a light chain variable region comprising an amino acid sequence having at least 90% identity to SEQ ID NO: 292;
   (iii) a heavy chain variable region comprising an amino acid sequence having at least 90% identity to SEQ ID NO: 116 and a light chain variable region comprising an amino acid sequence having at least 90% identity to SEQ ID NO: 293;
   (iv) a heavy chain variable region comprising an amino acid sequence having at least 90% identity to SEQ ID NO: 117 and a light chain variable region comprising an amino acid sequence having at least 90% identity to SEQ ID NO: 294;
   (v) a heavy chain variable region comprising an amino acid sequence having at least 90% identity to SEQ ID NO: 118 and a light chain variable region comprising an amino acid sequence having at least 90% identity to SEQ ID NO: 295;
   (vi) a heavy chain variable region comprising an amino acid sequence having at least 90% identity to SEQ ID NO: 119 and a light chain variable region comprising an amino acid sequence having at least 90% identity to SEQ ID NO: 296;
   (vii) a heavy chain variable region comprising an amino acid sequence having at least 90% identity to SEQ ID NO: 120 and a light chain variable region comprising an amino acid sequence having at least 90% identity to SEQ ID NO: 297;
   (viii) a heavy chain variable region comprising an amino acid sequence having at least 90% identity to SEQ ID NO: 121 and a light chain variable region comprising an amino acid sequence having at least 90% identity to SEQ ID NO: 298;
   (ix) a heavy chain variable region comprising an amino acid sequence having at least 90% identity to SEQ ID NO: 122 and a light chain variable region comprising an amino acid sequence having at least 90% identity to SEQ ID NO: 299;
   (x) a heavy chain variable region comprising an amino acid sequence having at least 90% identity to SEQ ID NO: 123 and a light chain variable region comprising an amino acid sequence having at least 90% identity to SEQ ID NO: 300;
   (xi) a heavy chain variable region comprising an amino acid sequence having at least 90% identity to SEQ ID NO: 124 and a light chain variable region comprising an amino acid sequence having at least 90% identity to SEQ ID NO: 301;
   (xii) a heavy chain variable region comprising an amino acid sequence having at least 90% identity to SEQ ID NO: 125 and a light chain variable region comprising an amino acid sequence having at least 90% identity to SEQ ID NO: 302;
   (xiii) a heavy chain variable region comprising an amino acid sequence having at least 90% identity to SEQ ID NO: 126 and a light chain variable region comprising an amino acid sequence having at least 90% identity to SEQ ID NO: 303;
   (xiv) a heavy chain variable region comprising an amino acid sequence having at least 90% identity to SEQ ID NO: 127 and a light chain variable region comprising an amino acid sequence having at least 90% identity to SEQ ID NO: 304;
   (xv) a heavy chain variable region comprising an amino acid sequence having at least 90% identity to SEQ ID NO: 128 and a light chain variable region comprising an amino acid sequence having at least 90% identity to SEQ ID NO: 305;
   (xvi) a heavy chain variable region comprising an amino acid sequence having at least 90% identity to SEQ ID NO: 129 and a light chain variable region comprising an amino acid sequence having at least 90% identity to SEQ ID NO: 306;
   (xvii) a heavy chain variable region comprising an amino acid sequence having at least 90% identity to SEQ ID NO: 130 and a light chain variable region comprising an amino acid sequence having at least 90% identity to SEQ ID NO: 307;
   (xviii) a heavy chain variable region comprising an amino acid sequence having at least 90% identity to SEQ ID NO: 131 and a light chain variable region comprising an amino acid sequence having at least 90% identity to SEQ ID NO: 308;
   (xix) a heavy chain variable region comprising an amino acid sequence having at least 90% identity to SEQ ID NO: 132 and a light chain variable region comprising an amino acid sequence having at least 90% identity to SEQ ID NO: 309;
   (xx) a heavy chain variable region comprising an amino acid sequence having at least 90% identity to SEQ ID NO: 133 and a light chain variable region comprising an amino acid sequence having at least 90% identity to SEQ ID NO: 310;
   (xxi) a heavy chain variable region comprising an amino acid sequence having at least 90% identity to SEQ ID NO: 134 and a light chain variable region comprising an amino acid sequence having at least 90% identity to SEQ ID NO: 311;
   (xxii) a heavy chain variable region comprising an amino acid sequence having at least 90% identity to SEQ ID NO: 135 and a light chain variable region comprising an amino acid sequence having at least 90% identity to SEQ ID NO: 312;
   (xxiii) a heavy chain variable region comprising an amino acid sequence having at least 90% identity to SEQ ID NO: 136 and a light chain variable region comprising an amino acid sequence having at least 90% identity to SEQ ID NO: 313;
   (xxiv) a heavy chain variable region comprising an amino acid sequence having at least 90% identity to SEQ ID NO: 137 and a light chain variable region comprising an amino acid sequence having at least 90% identity to SEQ ID NO: 314;
   (xxv) a heavy chain variable region comprising an amino acid sequence having at least 90% identity to SEQ ID NO: 149 and a light chain variable region comprising an amino acid sequence having at least 90% identity to SEQ ID NO: 326;
   (xxvi) a heavy chain variable region comprising an amino acid sequence having at least 90% identity to SEQ ID NO: 156 and a light chain variable region comprising an amino acid sequence having at least 90% identity to SEQ ID NO: 333;
   (xxvii) a heavy chain variable region comprising an amino acid sequence having at least 90% identity to SEQ ID NO: 158 and a light chain variable region comprising an amino acid sequence having at least 90% identity to SEQ ID NO: 335; or
   (xxviii) a heavy chain variable region comprising an amino acid sequence having at least 90% identity to SEQ ID NO: 143 and a light chain variable region comprising an amino acid sequence having at least 90% identity to SEQ ID NO: 320.
6. The antibody or fragment thereof of any one of embodiments 1-5, wherein the antibody comprises:
   (i) a heavy chain variable region comprising an amino acid sequence according to SEQ ID NO: 114 and a light chain variable region comprising an amino acid sequence according to SEQ ID NO: 291;
   (ii) a heavy chain variable region comprising an amino acid sequence according to SEQ ID NO: 115 and a light chain variable region comprising an amino acid sequence according to SEQ ID NO: 292;
   (iii) a heavy chain variable region comprising an amino acid sequence according to SEQ ID NO: 116 and a light chain variable region comprising an amino acid sequence according to SEQ ID NO: 293;
   (iv) a heavy chain variable region comprising an amino acid sequence according to SEQ ID NO: 117 and a light chain variable region comprising an amino acid sequence according to SEQ ID NO: 294;
   (v) a heavy chain variable region comprising an amino acid sequence according to SEQ ID NO: 118 and a light chain variable region comprising an amino acid sequence according to SEQ ID NO: 295;
   (vi) a heavy chain variable region comprising an amino acid sequence according to SEQ ID NO: 119 and a light chain variable region comprising an amino acid sequence according to SEQ ID NO: 296;
   (vii) a heavy chain variable region comprising an amino acid sequence according to SEQ ID NO: 120 and a light chain variable region comprising an amino acid sequence according to SEQ ID NO: 297;
   (viii) a heavy chain variable region comprising an amino acid sequence according to SEQ ID NO: 121 and a light chain variable region comprising an amino acid sequence according to SEQ ID NO: 298;
   (ix) a heavy chain variable region comprising an amino acid sequence according to SEQ ID NO: 122 and a light chain variable region comprising an amino acid sequence according to SEQ ID NO: 299;
   (x) a heavy chain variable region comprising an amino acid sequence according to SEQ ID NO: 123 and a light chain variable region comprising an amino acid sequence according to SEQ ID NO: 300;
   (xi) a heavy chain variable region comprising an amino acid sequence according to SEQ ID NO: 124 and a light chain variable region comprising an amino acid sequence according to SEQ ID NO: 301;
   (xii) a heavy chain variable region comprising an amino acid sequence according to SEQ ID NO: 125 and a light chain variable region comprising an amino acid sequence according to SEQ ID NO: 302;
   (xiii) a heavy chain variable region comprising an amino acid sequence according to SEQ ID NO: 126 and a light chain variable region comprising an amino acid sequence according to SEQ ID NO: 303;
   (xiv) a heavy chain variable region comprising an amino acid sequence according to SEQ ID NO: 127 and a light chain variable region comprising an amino acid sequence according to SEQ ID NO: 304;
   (xv) a heavy chain variable region comprising an amino acid sequence according to SEQ ID NO: 128 and a light chain variable region comprising an amino acid sequence according to SEQ ID NO: 305;
   (xvi) a heavy chain variable region comprising an amino acid sequence according to SEQ ID NO: 129 and a light chain variable region comprising an amino acid sequence according to SEQ ID NO: 306;
   (xvii) a heavy chain variable region comprising an amino acid sequence according to SEQ ID NO: 130 and a light chain variable region comprising an amino acid sequence according to SEQ ID NO: 307;
   (xviii) a heavy chain variable region comprising an amino acid sequence according to SEQ ID NO: 131 and a light chain variable region comprising an amino acid sequence according to SEQ ID NO: 308;
   (xix) a heavy chain variable region comprising an amino acid sequence according to SEQ ID NO: 132 and a light chain variable region comprising an amino acid sequence according to SEQ ID NO: 309;
   (xx) a heavy chain variable region comprising an amino acid sequence according to SEQ ID NO: 133 and a light chain variable region comprising an amino acid sequence according to SEQ ID NO: 310;
   (xxi) a heavy chain variable region comprising an amino acid sequence according to SEQ ID NO: 134 and a light chain variable region comprising an amino acid sequence according to SEQ ID NO: 311;
   (xxii) a heavy chain variable region comprising an amino acid sequence according to SEQ ID NO: 135 and a light chain variable region comprising an amino acid sequence according to SEQ ID NO: 312;
   (xxiii) a heavy chain variable region comprising an amino acid sequence according to SEQ ID NO: 136 and a light chain variable region comprising an amino acid sequence according to SEQ ID NO: 313;
   (xxiv) a heavy chain variable region comprising an amino acid sequence according to SEQ ID NO: 137 and a light chain variable region comprising an amino acid sequence according to SEQ ID NO: 314;
   (xxv) a heavy chain variable region comprising an amino acid sequence according to SEQ ID NO: 149 and a light chain variable region comprising an amino acid sequence according to SEQ ID NO: 326;
   (xxvi) a heavy chain variable region comprising an amino acid sequence according to SEQ ID NO: 156 and a light chain variable region comprising an amino acid sequence according to SEQ ID NO: 333;
   (xxvii) a heavy chain variable region comprising an amino acid sequence according to SEQ ID NO: 158 and a light chain variable region comprising an amino acid sequence according to SEQ ID NO: 335; or
   (xxviii) a heavy chain variable region comprising an amino acid sequence according to SEQ ID NO: 143 and a light chain variable region comprising an amino acid sequence according to SEQ ID NO: 320.
7. The antibody or fragment thereof of any one of embodiments 1-6, wherein the antibody or fragment thereof is a monoclonal antibody, a Fab, F(ab′)₂, Fab′, scFv, or a single domain antibody (sdAb).
8. The antibody or fragment thereof of any one of embodiments 1-6, wherein the antibody comprises a human IgG1 or IgG4 domain.
9. The antibody or fragment thereof of embodiment 8, comprising an IgG4 domain having a constant heavy domain according to SEQ ID NO: 1441 and a constant light domain according to SEQ ID NO: 1442.
10. The antibody or fragment thereof of embodiment 8, wherein the antibody comprises a human IgG4 domain comprising a S241P mutation at amino acid residue 241 and an L248E mutation at amino acid residue 248, wherein the numbering of the residues is that of the Kabat numbering system.
11. The antibody or fragment thereof of embodiment 10, comprising an IgG4 domain having a constant heavy domain according to SEQ ID NO: 1440 and a constant light domain according to SEQ ID NO: 1442.
12. The antibody or fragment thereof of any one embodiments 1-9, comprising a heavy chain amino acid sequence of any one of SEQ ID NOs: 892-914, 926, 933, 935, and 920 and a light chain amino acid sequence of any one of SEQ ID NOs: 1029-1051, 1063, 1070, 1072, and 1057.
13. The antibody or fragment thereof of any one of embodiments 1-8 or 10-11, comprising a heavy chain amino acid sequence of any one of SEQ ID NOs: 618-640, 652, 659, 661, and 646 and a light chain amino acid sequence of any one of SEQ ID NOs: 755-777, 789, 796, 798, and 783.
14. The antibody or fragment thereof of any one of embodiments 1-6, comprising an amino acid sequence of any one of SEQ ID NOs: 481-503 and 515, 522, 524, and 509.
15. The antibody or fragment thereof of any one of embodiments 1-14, wherein the antibody has a binding affinity for SCF of 50 nM or less.
16. The antibody or fragment thereof of any one of embodiments 1-14, wherein the antibody has a binding affinity for SCF of 10 nM or less.
17. The antibody or fragment thereof of any one of embodiments 1-14, wherein the antibody has a binding affinity for SCF of 5 nM or less.
18. The antibody or fragment thereof of any one of embodiments 1-17, wherein the antibody or fragment thereof blocks the interaction between SCF and c-Kit.
19. The antibody or fragment thereof of any one of embodiments 1-18 wherein the antibody or fragment thereof causes internalization of SCF.
20. The antibody or fragment thereof of any one of embodiments 1-19, wherein the antibody specifically binds to SCF248.
21. The antibody or fragment thereof of any one of embodiments 1-20, wherein the antibody does not bind to SCF220.
22. An isolated nucleic acid molecule encoding the antibody or fragment thereof of any one of embodiments 1-21.
23. An expression vector comprising a nucleic acid segment encoding the antibody or fragment thereof of any one of embodiments 1-21.
24. A recombinant host cell comprising the expression vector of embodiment 23.
25. A method for inhibiting inflammation or fibrosis in a subject in need thereof, the method comprising administering to the subject an antibody or fragment thereof according to any one of embodiments 1-21.
26. A method for treating a chronic inflammatory disease or a fibrotic disease in a subject in need thereof, the method comprising administering to the subject an antibody according to any one of embodiments 1-21.
27. The method of embodiment 26, wherein the chronic inflammatory disease or fibrotic disease is selected from the group consisting of urticaria, atopic dermatitis, non-alcoholic steatohepatitis (NASH), primary sclerosing cholangitis, pulmonary fibrosis, chronic obstructive pulmonary disease (COPD), acute respiratory distress syndrome (ARDS), cystic fibrosis, peribronchial fibrosis, hypersentitivity pneumonitis, asthma, bleomycin lung, scleroderma, liver cirrhosis, endomyocardial fibrosis, fibromyalgia, eosinophilic esophagitis, inflammatory bowel disease (IBD), chronic kidney disease (CKD), end stage renal disease (ERSD), renal fibrosis, glomerulonephritis, and nephropathy.
28. The antibody or fragment thereof of any one of embodiments 1-21, wherein the antibody binds to SCF248 or a fragment thereof with a higher absorbance for binding than an antibody having a heavy chain CDR1, CDR2, and CDR3 according to SEQ ID Nos: 14, 15, and 16 respectively; and a light chain CDR1, CDR2, and CDR3 according to SEQ ID NOs: 17, 18, and 19 at a particular antibody concentration.
29. The antibody or fragment thereof of any one of embodiments 1-21, wherein SCF248 or a fragment thereof comprises a polypeptide having the sequence of SEQ ID NO: 479.
30. The antibody or fragment thereof of any one of embodiments 1-21, wherein the antibody or fragment thereof reduces CCL2 expression in a cell by at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, or at least about 95% more than an antibody or a fragment thereof having a heavy chain CDR1, CDR2, and CDR3 according to SEQ ID Nos: 14, 15, and 16 respectively; and a light chain CDR1, CDR2, and CDR3 according to SEQ ID NOs: 17, 18, and 19.
31. The antibody or fragment thereof of any one of embodiments 1-21, wherein the antibody or fragment thereof reduces TGFβ expression in a cell by at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, or at least about 95% more than an antibody or a fragment thereof having a heavy chain CDR1, CDR2, and CDR3 according to SEQ ID Nos: 14, 15, and 16 respectively; and a light chain CDR1, CDR2, and CDR3 according to SEQ ID NOs: 17, 18, and 19.
32. The antibody or fragment thereof of any one of embodiments 1-21, wherein the antibody or fragment thereof has an about 11-fold to about 65-fold improved binding affinity for SCF248 or a fragment thereof compared to an antibody or a fragment thereof having a heavy chain CDR1, CDR2, and CDR3 according to SEQ ID Nos: 14, 15, and 16 respectively; and a light chain CDR1, CDR2, and CDR3 according to SEQ ID NOs: 17, 18, and 19.
33. The antibody or fragment thereof of embodiment 32, wherein the SCF248 or a fragment thereof comprises a polypeptide having the sequence of SEQ ID NO: 479.
34. The antibody or fragment thereof of embodiment 32 or 33, wherein the antibody or fragment thereof has a binding affinity that is at least 11-fold, at least 12-fold, at least 13-fold, at least 14-fold, at least 15-fold, at least 16-fold, at least 17-fold, at least 18-fold, at least 19-fold, at least 20-fold, at least 21-fold, at least 22-fold, at least 23-fold, at least 24-fold, at least 25-fold, at least 26-fold, at least 27-fold, at least 28-fold, at least 29-fold, at least 30-fold, at least 31-fold, at least 32-fold, at least 33-fold, at least 34-fold, at least 35-fold, at least 36-fold, at least 37-fold, at least 38-fold, at least 39-fold, at least 40-fold, at least 41-fold, at least 42-fold, at least 43-fold, at least 44-fold, at least 45-fold, at least 46-fold, at least 47-fold, at least 48-fold, at least 49-fold, at least 50-fold, at least 51-fold, at least 52-fold, at least 53-fold, at least 54-fold, at least 55-fold, at least 56-fold, at least 57-fold, at least 58-fold, at least 59-fold, at least 60-fold, at least 61-fold, at least 62-fold, at least 63-fold, at least 64-fold, or at least 65-fold improved.

### INCORPORATION BY REFERENCE

Publications, patents and patent applications cited herein are specifically incorporated by reference in their entireties. While the described invention has been described with reference to the specific embodiments thereof it should be understood by those skilled in the art that various changes may be made and equivalents may be substituted without departing from the true spirit and scope of the invention. In addition, many modifications may be made to adopt a particular situation, material, composition of matter, process, process step or steps, to the objective spirit and scope of the described invention. All such modifications are intended to be within the scope of the claims appended hereto.

1. An antibody or fragment thereof that specifically binds to stem cell factor (SCF), wherein the antibody comprises a heavy chain and a light chain, the heavy and light chain each comprising three complementarity determining regions (CDRs), comprising:
(i) a heavy chain CDR1 comprising an amino acid sequence according to SEQ ID NO: 1 (SX₂X₃MN, wherein X₂ is Q, N, or Y; and X₃ is W or Y);
(ii) a heavy chain CDR2 comprising an amino acid sequence according to SEQ ID NO: 2 (QIYPX₅DX₇DX₉HX₁₁NX₁₃KFX₁₆X₁₇, wherein X₅ is E, G, D, or L; X₇ is G, D or N; X₉ is T or I; X₁₁ is M or Y; X₁₃ is G, D, or E; X₁₁ is K, R, N, E, or D; and X₁₇ is G or T);
(iii) a heavy chain CDR3 comprising an amino acid sequence according to SEQ ID NO: 3 (X₁NWX₄GSY, wherein X₁ is S or A; and X₄ is V or D);
(iv) a light chain CDR1 comprising an amino acid sequence according to SEQ ID NO: 4 (X₁X₂SQSLLX₈X₉DGNTYLN, wherein X₁ is K or H; X₂ is S or A; X₈ is E or D; and X₉ is S, E, Q, A, or G);
(v) a light chain CDR2 comprising an amino acid sequence according to SEQ ID NO: 5 (LVX₃RX₅DX₇, wherein X₃ is D, N, or S; X₅ is L or R; and X₇ is I, D, S, or L); and
(vi) a light chain CDR3 comprising an amino acid sequence according to SEQ ID NO: 6 (WQGX₄X₅LPQT, wherein X₄ is T or S; and X₅ is D or H).

2. The antibody or fragment thereof of claim 1, comprising:
(i) a heavy chain CDR1 comprising an amino acid sequence according to SEQ ID NO: 7 (SX₂WMN, wherein X₂ is Q or N);
(ii) a heavy chain CDR2 comprising an amino acid sequence according to SEQ ID NO: 8 (QIYPX₅DX₇DX₉HX₁₁NX₁₃KFKX₁₇, wherein X₅ is E, G, or D; X₇ is G or D; X₉ is T or I; X₁₃ is G or D; X₁₁ is M or Y; and X₁₇ is G or T);
(iii) a heavy chain CDR3 comprising an amino acid sequence according to SEQ ID NO: 9 (SNWX₄GSY, wherein X₄ is V or D);
(iv) a light chain CDR1 comprising an amino acid sequence according to SEQ ID NO: 10 (KSSQSLLEX₉DGNTYLN, wherein X₉ is S, E, Q, or A);
(v) a light chain CDR2 comprising an amino acid sequence according to SEQ ID NO: 11 (LVX₃RLDX₇, wherein X₃ is D or N; X₇ is I, D, S, or L); and
(vi) a light chain CDR3 according to SEQ ID NO: 6 (WQGX₄X₅LPQT, wherein X₄ is T or S; and X₅ is D or H).

3. The antibody or fragment thereof of claim 1, comprising:
(i) a heavy chain CDR1, CDR2, and CDR3 according to SEQ ID Nos: 22, 26, and 16, respectively; and a light chain CDR1, CDR2, and CDR3 according to SEQ ID NOs: 71, 90, and 111;
(ii) a heavy chain CDR1, CDR2, and CDR3 according to SEQ ID Nos: 23, 27, and 16, respectively; and a light chain CDR1, CDR2, and CDR3 according to SEQ ID NOs: 71, 91, and 111;
(iii) a heavy chain CDR1, CDR2, and CDR3 according to SEQ ID Nos: 22, 26, and 67, respectively; and a light chain CDR1, CDR2, and CDR3 according to SEQ ID NOs: 71, 92, and 111;
(iv) a heavy chain CDR1, CDR2, and CDR3 according to SEQ ID Nos: 22, 27, and 16, respectively; and a light chain CDR1, CDR2, and CDR3 according to SEQ ID NOs: 71, 92, and 111;
(v) a heavy chain CDR1, CDR2, and CDR3 according to SEQ ID Nos: 23, 28, and 16, respectively; and a light chain CDR1, CDR2, and CDR3 according to SEQ ID NOs: 72, 92, and 111;
(vi) a heavy chain CDR1, CDR2, and CDR3 according to SEQ ID Nos: 22, 27, and 16, respectively; and a light chain CDR1, CDR2, and CDR3 according to SEQ ID NOs: 71, 93, and 112;
(vii) a heavy chain CDR1, CDR2, and CDR3 according to SEQ ID Nos: 22, 28, and 16, respectively; and a light chain CDR1, CDR2, and CDR3 according to SEQ ID NOs: 73, 92, and 111;
(viii) a heavy chain CDR1, CDR2, and CDR3 according to SEQ ID Nos: 22, 29, and 16, respectively; and a light chain CDR1, CDR2, and CDR3 according to SEQ ID NOs: 73, 92, and 111;
(ix) a heavy chain CDR1, CDR2, and CDR3 according to SEQ ID Nos: 22, 30, and 16, respectively; and a light chain CDR1, CDR2, and CDR3 according to SEQ ID NOs: 71, 92, and 111;
(x) a heavy chain CDR1, CDR2, and CDR3 according to SEQ ID Nos: 22, 31, and 16, respectively; and a light chain CDR1, CDR2, and CDR3 according to SEQ ID NOs: 71, 92, and 112;
(xi) a heavy chain CDR1, CDR2, and CDR3 according to SEQ ID Nos: 22, 32, and 16, respectively; and a light chain CDR1, CDR2, and CDR3 according to SEQ ID NOs: 73, 93, and 111;
(xii) a heavy chain CDR1, CDR2, and CDR3 according to SEQ ID Nos: 22, 33, and 16, respectively; and a light chain CDR1, CDR2, and CDR3 according to SEQ ID NOs: 74, 92, and 111;
(xiii) a heavy chain CDR1, CDR2, and CDR3 according to SEQ ID Nos: 24, 34, and 16, respectively; and a light chain CDR1, CDR2, and CDR3 according to SEQ ID NOs: 73, 94, and 111;
(xiv) a heavy chain CDR1, CDR2, and CDR3 according to SEQ ID Nos: 23, 28, and 16, respectively; and a light chain CDR1, CDR2, and CDR3 according to SEQ ID NOs: 73, 94, and 111;
(xv) a heavy chain CDR1, CDR2, and CDR3 according to SEQ ID Nos: 22, 31, and 16, respectively; and a light chain CDR1, CDR2, and CDR3 according to SEQ ID NOs: 73, 92, and 111;
(xvi) a heavy chain CDR1, CDR2, and CDR3 according to SEQ ID Nos: 22, 28, and 16, respectively; and a light chain CDR1, CDR2, and CDR3 according to SEQ ID NOs: 73, 92, and 111;
(xvii) a heavy chain CDR1, CDR2, and CDR3 according to SEQ ID Nos: 22, 27, and 16, respectively; and a light chain CDR1, CDR2, and CDR3 according to SEQ ID NOs: 73, 92, and 111;
(xviii) a heavy chain CDR1, CDR2, and CDR3 according to SEQ ID Nos: 22, 35, and 16, respectively; and a light chain CDR1, CDR2, and CDR3 according to SEQ ID NOs: 74, 92, and 111;
(xix) a heavy chain CDR1, CDR2, and CDR3 according to SEQ ID Nos: 22, 34, and 16, respectively; and a light chain CDR1, CDR2, and CDR3 according to SEQ ID NOs: 71, 92, and 111;
(xx) a heavy chain CDR1, CDR2, and CDR3 according to SEQ ID Nos: 22, 27, and 16, respectively; and a light chain CDR1, CDR2, and CDR3 according to SEQ ID NOs: 75, 92, and 111;
(xxi) a heavy chain CDR1, CDR2, and CDR3 according to SEQ ID Nos: 24, 34, and 16, respectively; and a light chain CDR1, CDR2, and CDR3 according to SEQ ID NOs: 73, 92, and 111;
(xxii) a heavy chain CDR1, CDR2, and CDR3 according to SEQ ID Nos: 22, 36, and 16, respectively; and a light chain CDR1, CDR2, and CDR3 according to SEQ ID NOs: 71, 95, and 111;
(xxiii) a heavy chain CDR1, CDR2, and CDR3 according to SEQ ID Nos: 23, 28, and 16, respectively; and a light chain CDR1, CDR2, and CDR3 according to SEQ ID NOs: 73, 92, and 111;
(xxiv) a heavy chain CDR1, CDR2, and CDR3 according to SEQ ID Nos: 22, 28, and 16, respectively; and a light chain CDR1, CDR2, and CDR3 according to SEQ ID NOs: 73, 96, and 111;
(xxv) a heavy chain CDR1, CDR2, and CDR3 according to SEQ ID Nos: 22, 34, and 16, respectively; and a light chain CDR1, CDR2, and CDR3 according to SEQ ID NOs: 71, 95, and 111;
(xxvi) a heavy chain CDR1, CDR2, and CDR3 according to SEQ ID Nos: 22, 34, and 16, respectively; and a light chain CDR1, CDR2, and CDR3 according to SEQ ID NOs: 79, 90, and 111;
(xxvii) a heavy chain CDR1, CDR2, and CDR3 according to SEQ ID Nos: 22, 34, and 16, respectively; and a light chain CDR1, CDR2, and CDR3 according to SEQ ID NOs: 75, 92, and 111; or
(xxviii) a heavy chain CDR1, CDR2, and CDR3 according to SEQ ID Nos: 22, 27, and 16, respectively; and a light chain CDR1, CDR2, and CDR3 according to SEQ ID NOs: 73, 98, and 111.

4. The antibody or fragment thereof of claim 1, wherein the antibody or fragment thereof comprises:
(i) a heavy chain variable region comprising an amino acid sequence having at least 80% identity to SEQ ID NO: 114 and a light chain variable region comprising an amino acid sequence having at least 80% identity to SEQ ID NO: 291;
(ii) a heavy chain variable region comprising an amino acid sequence having at least 80% identity to SEQ ID NO: 115 and a light chain variable region comprising an amino acid sequence having at least 80% identity to SEQ ID NO: 292;
(iii) a heavy chain variable region comprising an amino acid sequence having at least 80% identity to SEQ ID NO: 116 and a light chain variable region comprising an amino acid sequence having at least 80% identity to SEQ ID NO: 293;
(iv) a heavy chain variable region comprising an amino acid sequence having at least 80% identity to SEQ ID NO: 117 and a light chain variable region comprising an amino acid sequence having at least 80% identity to SEQ ID NO: 294;
(v) a heavy chain variable region comprising an amino acid sequence having at least 80% identity to SEQ ID NO: 118 and a light chain variable region comprising an amino acid sequence having at least 80% identity to SEQ ID NO: 295;
(vi) a heavy chain variable region comprising an amino acid sequence having at least 80% identity to SEQ ID NO: 119 and a light chain variable region comprising an amino acid sequence having at least 80% identity to SEQ ID NO: 296;
(vii) a heavy chain variable region comprising an amino acid sequence having at least 80% identity to SEQ ID NO: 120 and a light chain variable region comprising an amino acid sequence having at least 80% identity to SEQ ID NO: 297;
(viii) a heavy chain variable region comprising an amino acid sequence having at least 80% identity to SEQ ID NO: 121 and a light chain variable region comprising an amino acid sequence having at least 80% identity to SEQ ID NO: 298;
(ix) a heavy chain variable region comprising an amino acid sequence having at least 80% identity to SEQ ID NO: 122 and a light chain variable region comprising an amino acid sequence having at least 80% identity to SEQ ID NO: 299;
(x) a heavy chain variable region comprising an amino acid sequence having at least 80% identity to SEQ ID NO: 123 and a light chain variable region comprising an amino acid sequence having at least 80% identity to SEQ ID NO: 300;
(xi) a heavy chain variable region comprising an amino acid sequence having at least 80% identity to SEQ ID NO: 124 and a light chain variable region comprising an amino acid sequence having at least 80% identity to SEQ ID NO: 301;
(xii) a heavy chain variable region comprising an amino acid sequence having at least 80% identity to SEQ ID NO: 125 and a light chain variable region comprising an amino acid sequence having at least 80% identity to SEQ ID NO: 302;
(xiii) a heavy chain variable region comprising an amino acid sequence having at least 80% identity to SEQ ID NO: 126 and a light chain variable region comprising an amino acid sequence having at least 80% identity to SEQ ID NO: 303;
(xiv) a heavy chain variable region comprising an amino acid sequence having at least 80% identity to SEQ ID NO: 127 and a light chain variable region comprising an amino acid sequence having at least 80% identity to SEQ ID NO: 304;
(xv) a heavy chain variable region comprising an amino acid sequence having at least 80% identity to SEQ ID NO: 128 and a light chain variable region comprising an amino acid sequence having at least 80% identity to SEQ ID NO: 305;
(xvi) a heavy chain variable region comprising an amino acid sequence having at least 80% identity to SEQ ID NO: 129 and a light chain variable region comprising an amino acid sequence having at least 80% identity to SEQ ID NO: 306;
(xvii) a heavy chain variable region comprising an amino acid sequence having at least 80% identity to SEQ ID NO: 130 and a light chain variable region comprising an amino acid sequence having at least 80% identity to SEQ ID NO: 307;
(xviii) a heavy chain variable region comprising an amino acid sequence having at least 80% identity to SEQ ID NO: 131 and a light chain variable region comprising an amino acid sequence having at least 80% identity to SEQ ID NO: 308;
(xix) a heavy chain variable region comprising an amino acid sequence having at least 80% identity to SEQ ID NO: 132 and a light chain variable region comprising an amino acid sequence having at least 80% identity to SEQ ID NO: 309;
(xx) a heavy chain variable region comprising an amino acid sequence having at least 80% identity to SEQ ID NO: 133 and a light chain variable region comprising an amino acid sequence having at least 80% identity to SEQ ID NO: 310;
(xxi) a heavy chain variable region comprising an amino acid sequence having at least 80% identity to SEQ ID NO: 134 and a light chain variable region comprising an amino acid sequence having at least 80% identity to SEQ ID NO: 311;
(xxii) a heavy chain variable region comprising an amino acid sequence having at least 80% identity to SEQ ID NO: 135 and a light chain variable region comprising an amino acid sequence having at least 80% identity to SEQ ID NO: 312;
(xxiii) a heavy chain variable region comprising an amino acid sequence having at least 80% identity to SEQ ID NO: 136 and a light chain variable region comprising an amino acid sequence having at least 80% identity to SEQ ID NO: 313;
(xxiv) a heavy chain variable region comprising an amino acid sequence having at least 80% identity to SEQ ID NO: 137 and a light chain variable region comprising an amino acid sequence having at least 80% identity to SEQ ID NO: 314;
(xxv) a heavy chain variable region comprising an amino acid sequence having at least 80% identity to SEQ ID NO: 149 and a light chain variable region comprising an amino acid sequence having at least 80% identity to SEQ ID NO: 326;
(xxvi) a heavy chain variable region comprising an amino acid sequence having at least 80% identity to SEQ ID NO: 156 and a light chain variable region comprising an amino acid sequence having at least 80% identity to SEQ ID NO: 333;
(xxvii) a heavy chain variable region comprising an amino acid sequence having at least 80% identity to SEQ ID NO: 158 and a light chain variable region comprising an amino acid sequence having at least 80% identity to SEQ ID NO: 335; or
(xxviii) a heavy chain variable region comprising an amino acid sequence having at least 80% identity to SEQ ID NO: 143 and a light chain variable region comprising an amino acid sequence having at least 80% identity to SEQ ID NO: 320.

5. The antibody or fragment thereof of claim 1, wherein the antibody or fragment thereof comprises:
(i) a heavy chain variable region comprising an amino acid sequence having at least 90% identity to SEQ ID NO: 114 and a light chain variable region comprising an amino acid sequence having at least 90% identity to SEQ ID NO: 291;
(ii) a heavy chain variable region comprising an amino acid sequence having at least 90% identity to SEQ ID NO: 115 and a light chain variable region comprising an amino acid sequence having at least 90% identity to SEQ ID NO: 292;
(iii) a heavy chain variable region comprising an amino acid sequence having at least 90% identity to SEQ ID NO: 116 and a light chain variable region comprising an amino acid sequence having at least 90% identity to SEQ ID NO: 293;
(iv) a heavy chain variable region comprising an amino acid sequence having at least 90% identity to SEQ ID NO: 117 and a light chain variable region comprising an amino acid sequence having at least 90% identity to SEQ ID NO: 294;
(v) a heavy chain variable region comprising an amino acid sequence having at least 90% identity to SEQ ID NO: 118 and a light chain variable region comprising an amino acid sequence having at least 90% identity to SEQ ID NO: 295;
(vi) a heavy chain variable region comprising an amino acid sequence having at least 90% identity to SEQ ID NO: 119 and a light chain variable region comprising an amino acid sequence having at least 90% identity to SEQ ID NO: 296;
(vii) a heavy chain variable region comprising an amino acid sequence having at least 90% identity to SEQ ID NO: 120 and a light chain variable region comprising an amino acid sequence having at least 90% identity to SEQ ID NO: 297;
(viii) a heavy chain variable region comprising an amino acid sequence having at least 90% identity to SEQ ID NO: 121 and a light chain variable region comprising an amino acid sequence having at least 90% identity to SEQ ID NO: 298;
(ix) a heavy chain variable region comprising an amino acid sequence having at least 90% identity to SEQ ID NO: 122 and a light chain variable region comprising an amino acid sequence having at least 90% identity to SEQ ID NO: 299;
(x) a heavy chain variable region comprising an amino acid sequence having at least 90% identity to SEQ ID NO: 123 and a light chain variable region comprising an amino acid sequence having at least 90% identity to SEQ ID NO: 300;
(xi) a heavy chain variable region comprising an amino acid sequence having at least 90% identity to SEQ ID NO: 124 and a light chain variable region comprising an amino acid sequence having at least 90% identity to SEQ ID NO: 301;
(xii) a heavy chain variable region comprising an amino acid sequence having at least 90% identity to SEQ ID NO: 125 and a light chain variable region comprising an amino acid sequence having at least 90% identity to SEQ ID NO: 302;
(xiii) a heavy chain variable region comprising an amino acid sequence having at least 90% identity to SEQ ID NO: 126 and a light chain variable region comprising an amino acid sequence having at least 90% identity to SEQ ID NO: 303;
(xiv) a heavy chain variable region comprising an amino acid sequence having at least 90% identity to SEQ ID NO: 127 and a light chain variable region comprising an amino acid sequence having at least 90% identity to SEQ ID NO: 304;
(xv) a heavy chain variable region comprising an amino acid sequence having at least 90% identity to SEQ ID NO: 128 and a light chain variable region comprising an amino acid sequence having at least 90% identity to SEQ ID NO: 305;
(xvi) a heavy chain variable region comprising an amino acid sequence having at least 90% identity to SEQ ID NO: 129 and a light chain variable region comprising an amino acid sequence having at least 90% identity to SEQ ID NO: 306;
(xvii) a heavy chain variable region comprising an amino acid sequence having at least 90% identity to SEQ ID NO: 130 and a light chain variable region comprising an amino acid sequence having at least 90% identity to SEQ ID NO: 307;
(xviii) a heavy chain variable region comprising an amino acid sequence having at least 90% identity to SEQ ID NO: 131 and a light chain variable region comprising an amino acid sequence having at least 90% identity to SEQ ID NO: 308;
(xix) a heavy chain variable region comprising an amino acid sequence having at least 90% identity to SEQ ID NO: 132 and a light chain variable region comprising an amino acid sequence having at least 90% identity to SEQ ID NO: 309;
(xx) a heavy chain variable region comprising an amino acid sequence having at least 90% identity to SEQ ID NO: 133 and a light chain variable region comprising an amino acid sequence having at least 90% identity to SEQ ID NO: 310;
(xxi) a heavy chain variable region comprising an amino acid sequence having at least 90% identity to SEQ ID NO: 134 and a light chain variable region comprising an amino acid sequence having at least 90% identity to SEQ ID NO: 311;
(xxii) a heavy chain variable region comprising an amino acid sequence having at least 90% identity to SEQ ID NO: 135 and a light chain variable region comprising an amino acid sequence having at least 90% identity to SEQ ID NO: 312;
(xxiii) a heavy chain variable region comprising an amino acid sequence having at least 90% identity to SEQ ID NO: 136 and a light chain variable region comprising an amino acid sequence having at least 90% identity to SEQ ID NO: 313;
(xxiv) a heavy chain variable region comprising an amino acid sequence having at least 90% identity to SEQ ID NO: 137 and a light chain variable region comprising an amino acid sequence having at least 90% identity to SEQ ID NO: 314;
(xxv) a heavy chain variable region comprising an amino acid sequence having at least 90% identity to SEQ ID NO: 149 and a light chain variable region comprising an amino acid sequence having at least 90% identity to SEQ ID NO: 326;
(xxvi) a heavy chain variable region comprising an amino acid sequence having at least 90% identity to SEQ ID NO: 156 and a light chain variable region comprising an amino acid sequence having at least 90% identity to SEQ ID NO: 333;
(xxvii) a heavy chain variable region comprising an amino acid sequence having at least 90% identity to SEQ ID NO: 158 and a light chain variable region comprising an amino acid sequence having at least 90% identity to SEQ ID NO: 335; or
(xxviii) a heavy chain variable region comprising an amino acid sequence having at least 90% identity to SEQ ID NO: 143 and a light chain variable region comprising an amino acid sequence having at least 90% identity to SEQ ID NO: 320.

6. The antibody or fragment thereof of claim 1, wherein the antibody or fragment thereof comprises:
(i) a heavy chain variable region comprising an amino acid sequence according to SEQ ID NO: 114 and a light chain variable region comprising an amino acid sequence according to SEQ ID NO: 291;
(ii) a heavy chain variable region comprising an amino acid sequence according to SEQ ID NO: 115 and a light chain variable region comprising an amino acid sequence according to SEQ ID NO: 292;
(iii) a heavy chain variable region comprising an amino acid sequence according to SEQ ID NO: 116 and a light chain variable region comprising an amino acid sequence according to SEQ ID NO: 293;
(iv) a heavy chain variable region comprising an amino acid sequence according to SEQ ID NO: 117 and a light chain variable region comprising an amino acid sequence according to SEQ ID NO: 294;
(v) a heavy chain variable region comprising an amino acid sequence according to SEQ ID NO: 118 and a light chain variable region comprising an amino acid sequence according to SEQ ID NO: 295;
(vi) a heavy chain variable region comprising an amino acid sequence according to SEQ ID NO: 119 and a light chain variable region comprising an amino acid sequence according to SEQ ID NO: 296;
(vii) a heavy chain variable region comprising an amino acid sequence according to SEQ ID NO: 120 and a light chain variable region comprising an amino acid sequence according to SEQ ID NO: 297;
(viii) a heavy chain variable region comprising an amino acid sequence according to SEQ ID NO: 121 and a light chain variable region comprising an amino acid sequence according to SEQ ID NO: 298;
(ix) a heavy chain variable region comprising an amino acid sequence according to SEQ ID NO: 122 and a light chain variable region comprising an amino acid sequence according to SEQ ID NO: 299;
(x) a heavy chain variable region comprising an amino acid sequence according to SEQ ID NO: 123 and a light chain variable region comprising an amino acid sequence according to SEQ ID NO: 300;
(xi) a heavy chain variable region comprising an amino acid sequence according to SEQ ID NO: 124 and a light chain variable region comprising an amino acid sequence according to SEQ ID NO: 301;
(xii) a heavy chain variable region comprising an amino acid sequence according to SEQ ID NO: 125 and a light chain variable region comprising an amino acid sequence according to SEQ ID NO: 302;
(xiii) a heavy chain variable region comprising an amino acid sequence according to SEQ ID NO: 126 and a light chain variable region comprising an amino acid sequence according to SEQ ID NO: 303;
(xiv) a heavy chain variable region comprising an amino acid sequence according to SEQ ID NO: 127 and a light chain variable region comprising an amino acid sequence according to SEQ ID NO: 304;
(xv) a heavy chain variable region comprising an amino acid sequence according to SEQ ID NO: 128 and a light chain variable region comprising an amino acid sequence according to SEQ ID NO: 305;
(xvi) a heavy chain variable region comprising an amino acid sequence according to SEQ ID NO: 129 and a light chain variable region comprising an amino acid sequence according to SEQ ID NO: 306;
(xvii) a heavy chain variable region comprising an amino acid sequence according to SEQ ID NO: 130 and a light chain variable region comprising an amino acid sequence according to SEQ ID NO: 307;
(xviii) a heavy chain variable region comprising an amino acid sequence according to SEQ ID NO: 131 and a light chain variable region comprising an amino acid sequence according to SEQ ID NO: 308;
(xix) a heavy chain variable region comprising an amino acid sequence according to SEQ ID NO: 132 and a light chain variable region comprising an amino acid sequence according to SEQ ID NO: 309;
(xx) a heavy chain variable region comprising an amino acid sequence according to SEQ ID NO: 133 and a light chain variable region comprising an amino acid sequence according to SEQ ID NO: 310;
(xxi) a heavy chain variable region comprising an amino acid sequence according to SEQ ID NO: 134 and a light chain variable region comprising an amino acid sequence according to SEQ ID NO: 311;
(xxii) a heavy chain variable region comprising an amino acid sequence according to SEQ ID NO: 135 and a light chain variable region comprising an amino acid sequence according to SEQ ID NO: 312;
(xxiii) a heavy chain variable region comprising an amino acid sequence according to SEQ ID NO: 136 and a light chain variable region comprising an amino acid sequence according to SEQ ID NO: 313;
(xxiv) a heavy chain variable region comprising an amino acid sequence according to SEQ ID NO: 137 and a light chain variable region comprising an amino acid sequence according to SEQ ID NO: 314;
(xxv) a heavy chain variable region comprising an amino acid sequence according to SEQ ID NO: 149 and a light chain variable region comprising an amino acid sequence according to SEQ ID NO: 326;
(xxvi) a heavy chain variable region comprising an amino acid sequence according to SEQ ID NO: 156 and a light chain variable region comprising an amino acid sequence according to SEQ ID NO: 333;
(xxvii) a heavy chain variable region comprising an amino acid sequence according to SEQ ID NO: 158 and a light chain variable region comprising an amino acid sequence according to SEQ ID NO: 335; or
(xxviii) a heavy chain variable region comprising an amino acid sequence according to SEQ ID NO: 143 and a light chain variable region comprising an amino acid sequence according to SEQ ID NO: 320.

7. The antibody or fragment thereof of claim 1, wherein the antibody or fragment thereof is a monoclonal antibody, a Fab, F(ab′)₂, Fab′, scFv, or a single domain antibody (sdAb).

8. The antibody or fragment thereof of claim 1, wherein the antibody or fragment thereof comprises a human IgG1 or IgG4 domain.

9. The antibody or fragment thereof of claim 8, comprising an IgG4 domain having a constant heavy domain according to SEQ ID NO: 1441 and a constant light domain according to SEQ ID NO: 1442.

10. The antibody or fragment thereof of claim 8, wherein the antibody comprises a human IgG4 domain comprising a S241P mutation at amino acid residue 241 and an L248E mutation at amino acid residue 248, wherein the numbering of the residues is that of the Kabat numbering system.

11. The antibody or fragment thereof of claim 10, comprising an IgG4 domain having a constant heavy domain according to SEQ ID NO: 1440 and a constant light domain according to SEQ ID NO: 1442.

12. The antibody or fragment thereof of claim 1, comprising a heavy chain amino acid sequence of any one of SEQ ID NOs: 892-914, 926, 933, 935, and 920 and a light chain amino acid sequence of any one of SEQ ID NOs: 1029-1051, 1063, 1070, 1072, and 1057.

13. The antibody or fragment thereof of claim 1, comprising a heavy chain amino acid sequence of any one of SEQ ID NOs: 618-640, 652, 659, 661, and 646 and a light chain amino acid sequence of any one of SEQ ID NOs: 755-777, 789, 796, 798, and 783.

14. The antibody or fragment thereof of claim 1, comprising an amino acid sequence of any one of SEQ ID NOs: 481-503 and 515, 522, 524, and 509.

15. The antibody or fragment thereof of claim 1, wherein the antibody has a binding affinity for SCF of 50 nM or less.

16. The antibody or fragment thereof of claim 1, wherein the antibody has a binding affinity for SCF of 10 nM or less.

17. The antibody or fragment thereof of claim 1, wherein the antibody has a binding affinity for SCF of 5 nM or less.

18. The antibody or fragment thereof of claim 1, wherein the antibody or fragment thereof blocks the interaction between SCF and c-Kit.

19. The antibody or fragment thereof of claim 1, wherein the antibody or fragment thereof causes internalization of SCF.

20. The antibody or fragment thereof of claim 1, wherein the antibody or fragment thereof specifically binds to SCF248.

21. The antibody or fragment thereof of claim 1, wherein the antibody or fragment thereof does not bind to SCF220.

22. An isolated nucleic acid molecule encoding the antibody or fragment thereof of claim 1.

23. An expression vector comprising a nucleic acid segment encoding the antibody or fragment thereof of claim 1.

24. A recombinant host cell comprising the expression vector of claim 23.

25. A method for inhibiting inflammation or fibrosis in a subject in need thereof, the method comprising administering to the subject an antibody or fragment thereof according to claim 1.

26. A method for treating a chronic inflammatory disease or a fibrotic disease in a subject in need thereof, the method comprising administering to the subject an antibody according to claim 1.

27. The method of claim 26, wherein the chronic inflammatory disease or fibrotic disease is selected from the group consisting of urticaria, atopic dermatitis, non-alcoholic steatohepatitis (NASH), primary sclerosing cholangitis, pulmonary fibrosis, chronic obstructive pulmonary disease (COPD), acute respiratory distress syndrome (ARDS), cystic fibrosis, peribronchial fibrosis, hypersentitivity pneumonitis, asthma, bleomycin lung, scleroderma, liver cirrhosis, endomyocardial fibrosis, fibromyalgia, eosinophilic esophagitis, inflammatory bowel disease (IBD), chronic kidney disease (CKD), end stage renal disease (ERSD), renal fibrosis, glomerulonephritis, and nephropathy.
